(19) United States
(12) Patent Application Publication
(10) Pub. No.: US 2014/0378451 A1
(43) Pub. Date: Dec. 25, 2014

(54) Cannabinoid-2-Receptor Agonists

(71) Applicant: ISIS INNOVATION LIMITED, Oxford (GB)

(72) Inventors: Ivy Christou, Oxford (GB); Rebecca Lillian Cross, Oxford (GB); Matteo Gianella-Borradori, Oxford (GB); David Robert Greaves, Oxford (GB); Angela Jane Russell, Oxford (GB); Graham Michael Wynne, Oxford (GB)

(21) Appl. No.: 14/345,790

(22) PCT Filed: Sep. 19, 2012
(86) PCT No.: PCT/GB2012/052313

§ 371 Date: Mar. 19, 2014

(30) Foreign Application Priority Data
Sep. 19, 2011 (GB) 1116152.8

(51) Int. Cl. C07D 487/04

(52) U.S. Cl. CPC C07D 487/04
USPC 514/234.2; 514/243; 544/184; 544/112

(57) ABSTRACT

The present invention relates to cannabinoid-2-receptor (CB2R) agonist compounds. The present invention also relates to processes for the preparation of these compounds, to pharmaceutical compositions comprising them and to their use as therapeutic agents for the treatment and/or prevention of diseases or conditions in which CB2R stimulation is beneficial (especially inflammatory conditions).

### FIELD OF THE INVENTION

The present invention relates to compounds that are cannabinoid-2-receptor (CB2R) agonists. The present invention also relates to processes for the preparation of these compounds, to pharmaceutical compositions comprising them and to their use as therapeutic agents for the treatment and/or prevention of conditions in which the stimulation of CB2R is beneficial.

### BACKGROUND OF THE INVENTION

The endocannabinoid system (ECS) is an endogenous signalling system that has a modulating role in pain perception, gut motility, immune responses, food intake, energy metabolism and mood. The ECS consists of at least two distinct membrane receptors, along with the endocannabinoid ligands, the endocannabinoid membrane transporter and endocannabinoid metabolizing enzymes. Two cannabinoid receptors (CBR) are already known and designated as CB1R and CB2R while controversy exists on whether GPR55 is a third atypical endocannabinoid receptor. From the endocannabinoid ligands, the two main and most studied ligands are anandamide (AEA) and 2-arachidonoylglycerol (2-AG) while other putative endogenous ligands include N-palmitoylethanolamine (also known as palmitoylethanolamide).

The ECS system is multifaceted and its action depends on tissue location, cell type and functional selectivity of cannabinoid ligands. CB1R is predominantly expressed in the central nervous system but can be expressed in the periphery, while CB2R is predominantly expressed in the peripheral tissues and immune cells but has low expression in the brain (1).

Importantly, there is conservation of the receptors between species. Human and mouse CB1R proteins share 96% homology (2) while human and mouse CB2R protein share 80% homology and 87% similarity. Of the endocannabinoid ligands AEA is considered a partial agonist of the two receptors with greater selectivity for CB1 over CB2 while 2-AG is considered a full agonist at both CB1 and CB2 receptors (3). Recent evidence suggests that different full agonists may activate different signalling pathways at the cannabinoid receptors (4), a phenomenon referred to as functional selectivity. 2-AG can be produced from a wide range of cells including platelets (5), macrophages (5), astrocytes (6), spleen and many parts of the brain (7), while AEA has been shown to be synthesized from cells such as macrophages (8) and astrocytes (9) and in tissues such as the brain and spleen (10). Overall, key components of the ECS appear to be present, and under certain conditions activated, in all key cell types of the immune system. Taken together with its high conservation between species this suggests that the ECS could play an important role in the physiological reign of host immunity.

So far all studied endocannabinoid ligands have been shown to be synthesized ‘on demand’ by cleavage and metabolism of membrane lipid precursors. Studies on inflammatory responses suggest that stimulus-induced production of endocannabinoids and subsequent receptor activation may play a role in the regulation of immune responses in several pathological conditions. For example, the role of the ECS system has been investigated in in vivo models of neuroinflammatory disorders such as multiple sclerosis (11) or in diseases with an inflammatory component such as allergic contact dermatitis (12), colitis (13) and atherosclerosis (14).

The importance of the ECS in inflammation has been shown in several studies. Of particular interest is the study of Steffens et al, which demonstrated that in ApoE^{-/-} mice fed with a high fat diet (a mouse model of atherosclerosis), CB2R was present within human coronary atheromata and atherosclerotic lesions of mouse aortic arch and root but not in non-diseased arteries (14). Importantly, mice treated with Δ9-THC for 2-6 weeks showed significantly reduced progression of atherosclerotic lesions compared to controls and this effect was reversed by the CB2R-specific antagonist SR144528. The authors also showed a reduced macrophage infiltration in atherosclerotic lesions of Δ9-THC treated mice (14). In other published studies CB2R activation has been shown to induce monocyte chemotaxis (15), reduce monocyte chemotaxis to MCP-1/MIP-1α (15) and reduce macrophage chemotaxis to MCP-1, RANTES and fMLP (14, 16, 17). Consideration of the published literature suggests that CB2R appears to play an immuno-modulatory role in macrophage inflammatory responses. The role of CBIR is less clear and the role of GPR55 remains to be investigated.

There is a need for new and effective CB2R agonist compounds that could be useful for the treatment of diseases or conditions in which the stimulation of CB2R would be beneficial. In particular, such agonist compounds could be used as anti-inflammatory agents. Diseases or conditions that could be treated with such agents include any conditions in which inflammatory processes are involved, such as, for example, cardiovascular disease (e.g. atherosclerosis), inflammatory pain (both peripheral inflammatory pain and chronic inflammatory pain), allergies and the promotion of wound healing.

There is also a need for CB2R agonist compounds that demonstrate good selectivity for CB2R over CB1R and/or which do not penetrate the blood brain barrier to a significant extent.

It is therefore an objective of the present invention to provide potent new and effective CB2R agonist compounds.

It is a further objective of the present invention to provide compounds that demonstrate selectivity for CB2R over CB1R.

### SUMMARY OF THE INVENTION

The present invention resides in the identification of a novel series of compounds that function as CB2R agonists.

Therefore, in a first aspect, the present invention provides a compound of formula I as defined herein, or a pharmaceutically acceptable salt or solvate thereof.

In another aspect, the present invention provides a pharmaceutical composition comprising a compound of formula I as defined herein, or a pharmaceutically acceptable salt or solvate thereof, in admixture with a pharmaceutically acceptable diluent or carrier.

In another aspect, the present invention provides a compound of formula I as defined herein, or a pharmaceutically acceptable salt or solvate thereof, for use in therapy.

In another aspect, the present invention provides a method of treating a condition in which the stimulation of CB2R is beneficial (as defined herein), the method comprising administering a therapeutically effective amount of a compound of formula I as defined herein, or a pharmaceutically acceptable salt or solvate thereof, to a subject in need of such treatment.

In another aspect, the present invention provides a compound of formula I as defined herein, or a pharmaceutically acceptable salt or solvate thereof, for use in the treatment of a condition in which the stimulation of CB2R is beneficial (as defined herein).

In another aspect, the present invention provides the use of a compound of formula I as defined herein, or a pharmaceutically acceptable salt or solvate thereof, in the manufacture of a medicament for use in the treatment of a condition in which the stimulation of CB2R is beneficial (as defined herein).

In a particular aspect, the present invention provides a method of treating an inflammatory condition, the method comprising administering a therapeutically effective amount of a compound of formula I as defined herein, or a pharmaceutically acceptable salt or solvate thereof, to a subject in need of such treatment.

In another particular aspect, the present invention provides a compound of formula I as defined herein, or a pharmaceutically acceptable salt or solvate thereof, for use in the treatment of inflammation.

In another aspect, the present invention provides the use of a compound of formula I as defined herein, or a pharmaceutically acceptable salt or solvate thereof, in the manufacture of a medicament for use in the treatment of inflammation.

In another aspect, the present invention provides a compound of formula I as defined herein, or a pharmaceutically acceptable salt or solvate thereof, for use in the treatment of a condition in which the stimulation of CB2R is beneficial (as defined herein) in combination with one or more additional therapeutic agents.

In another aspect, the present invention provides the use of a compound of formula I as defined herein, or a pharmaceutically acceptable salt or solvate thereof, in the manufacture of medicament for use in the treatment of a condition in which the stimulation of CB2R is beneficial (as defined herein) in combination with one or more additional therapeutic agents.

In another aspect, the present invention provides a compound of formula I as defined herein, or a pharmaceutically acceptable salt or solvate thereof, for use as a CB2R agonist (in vitro or in vivo).

In another aspect, the present invention provides the use of a compound of formula I as defined herein, or a pharmaceutically acceptable salt or solvate thereof, in the manufacture of a medicament for use as an agonist of CB2R.

In another aspect, the present invention provides a method of stimulating CB2R in a cell, tissue or a subject, the method comprising administering a compound of formula I as defined herein, or a pharmaceutically acceptable salt or solvate thereof to said cell, tissue or subject.

The present invention further provides a method of synthesising a compound of formula I or a pharmaceutically acceptable salt or solvate thereof as defined herein.

In another aspect, the present invention provides a compound of formula I as defined herein, or a pharmaceutically acceptable salt or solvate thereof, obtainable by, or obtained by, or directly obtained by a method of synthesis defined herein.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity as well as the age, weight, etc., of the patient to be treated.

The term “(m-nC)” or “(m-nC) group” used alone or as a prefix, refers to any group having m to n carbon atoms.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl.

“(3-8C)cycloalkyl” means a hydrocarbon ring containing from 3 to 8 carbon atoms, for example, cyclopropyl, cyclobutyl, cyclopentyl, cyclohexyl, cycloheptyl or bicyclo[2.2.1]heptyl.

The term “halo” refers to fluoro, chloro, bromo and iodo.

The term “heterocyclyl”, “heterocyclic” or “heterocycle” means a non-aromatic saturated or partially saturated monocyclic or bicyclic heterocyclic ring system. The term heterocyclyl includes both monovalent species and divalent species. Monocyclic heterocyclic rings contain from about 3 to 10 (suitably from 4 to 7) ring atoms, with from 1 to 4 (suitably 1, 2 or 3) heteroatoms selected from nitrogen, oxygen or sulphur in the ring. Bicyclic heterocycles contain from 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulphur containing heterocycles include tetrahydrothienyl, dihydro-1,3-dithiol, tetrahydro-2H-thiopyran, and hexahydrothiepine. For heterocycles containing sulphur, the oxidized sulphur heterocycles containing SO or SO₂ groups are also included. Particular heterocyclyl groups are saturated monocyclic 4 to 7 membered heterocyclyls containing 1, 2 or 3 heteroatoms selected from nitrogen, oxygen or sulphur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable heterocyclic ring atom, such as via a carbon or nitrogen atom.

The term “heteroaryl” or “heteroaromatic” means an aromatic mono- or bi-cyclic ring incorporating one or more (for example 1-4, particularly 1, 2 or 3) heteroatoms selected from nitrogen, oxygen or sulphur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulphur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydrobenzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

The term “neutral heteroaryl” is used herein to refer to heteroaryl groups that are non-basic, i.e. are not amenable to protonation at physiological pH ranges. Suitable examples of neutral heteroaryls will be known to those skilled in the art and include thiophene, pyridazine, pyrazine, pyrimide, furan, oxazole, thiazole, benzothiophene, benzofuran and the like.

The term “aryl” means a cyclic or polycyclic aromatic ring having from 6 to 12 carbon atoms. The term aryl includes both monovalent species and divalent species. Examples of aryl groups include, but are not limited to, phenyl, biphenyl, naphthyl and the like. In a particular embodiment, an aryl is phenyl.

The term “optionally substituted” refers to either groups, structures or molecules that are substituted as well as those that are not substituted.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

According to a first aspect of the present invention there is provided a compound of formula I shown below

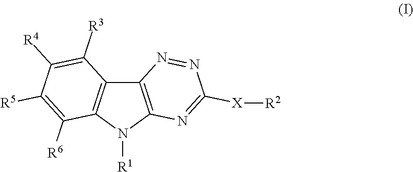

wherein:
X is —S—, —O—, —SO—, NR^{a} or —CH₂—;
R^{a} is hydrogen or (1-3C)alkyl;
R¹ is selected from hydrogen or a group of the formula:

X⁰—X¹-Q¹
wherein
X⁰ is absent or —(CH₂)ₙ—;
X¹ is absent —CO— or —SO₂—;
n is 1, 2 or 3;
when X¹ is absent or —CO— then Q¹ is selected from (1-5C)alkyl, (3-8C)cycloalkyl, aryl, a carbon-linked heterocyclyl, a carbon-linked heteroaryl or —NR⁷R⁸ where R⁷ and R⁸ are each independently selected from methyl or ethyl, or R⁷ and R⁸ are linked so that, together with the nitrogen atom to which they are attached, they form a 4-7 membered heterocyclic ring optionally comprising one, two or three additional heteroatoms selected from N, O or S;
when X¹ is —SO₂— then Q¹ is selected from (1-5C)alkyl, (3-8C)cycloalkyl, phenyl, thiophene or —NR⁷R⁸ (where R⁷ and R⁸ are each as defined above);
and wherein Q¹ is optionally substituted by one or more substituents selected from halo, trifluoromethyl, trifluoromethoxy, cyano, isocyano, nitro, hydroxy, mercapto, amino, formyl, carboxy, carbamoyl, ureido, (1-4C)alkyl, (2-4C)alkenyl, (2-4C)alkynyl, (1-4C)alkoxy, (1-4C)alkylthio, (1-4C)alkylsulphinyl, (1-4C)alkylsulphonyl, (1-4C)alkylamino, di-[(1-4C)alkyl]amino, (1-4C)alkoxycarbonyl, N-(1-4C)alkylcarbamoyl, N,N-di-[(1-4C)alkyl]carbamoyl, (2-4C)alkanoyl, (2-4C)alkanoyloxy, (2-4C)alkanoylamino, sulphamoyl, N-(1-4C)alkylsulphamoyl, N,N-di-[(1-4C)alkyl]sulphamoyl or a phenyl ring which is optionally further substituted by halo, methoxy, ethoxy, methyl, ethyl, cyano or hydroxy;
R² is selected from one of the following options:
(i) when R¹ is hydrogen, R² is selected from (3-5C)alkyl, (3-5C)alkenyl, (3-5C)alkynyl, aryl, (3-8C)cycloalkyl, thienyl or a group of the formula:

—CH₂-Q²
wherein Q² is selected from:
(3-8C)cycloalkyl which is optionally substituted with cyano, nitro, fluoro or methyl;
phenyl which is substituted in the ortho or para position (relative to the point of attachment to the —CH₂— group) by cyano, nitro, methyl, —CO₂H and tetrazole and optionally further substituted with cyano, nitro, fluoro, or methyl;
naphthyl which is optionally substituted with cyano, nitro, fluoro or methyl; or
thiophene which is optionally substituted with cyano, nitro, fluoro or methyl;
(ii) when R¹ is a substituent group other than hydrogen, R² is selected from (3-5C)alkyl, (3-5C)alkenyl, (3-5C)alkynyl, aryl, (3-8C)cycloalkyl, thienyl or a group of the formula:

—CH₂-Q³
wherein Q³ is selected from phenyl, (3-8C)cycloalkyl, naphthyl or a neutral heteroaryl, each of which is optionally substituted by cyano, nitro, halo, methyl, trifluoromethyl, trifluoromethoxy, isocyano, hydroxy, mercapto, amino, carboxy, carbamoyl, methoxy, methylthio, methylsulphinyl, methylsulphonyl, methylamino, or di-methylamino;
R³, R⁴, R⁵ or R⁶ are each independently selected from hydrogen, halo, (1-5C)alkyl, (3-6C)cycloalkyl, (3-6C)cycloalkyl(1-2C)alkyl, thiophene or aryl, wherein the aryl ring is optionally substituted by halo, methoxy, ethoxy, methyl, ethyl, cyano or hydroxy;
with the proviso that R⁴, R⁵ or R⁶ are all hydrogen when R¹ is hydrogen;
   or a pharmaceutically acceptable salt or solvate thereof.

In a second aspect, the present invention provides a compound of formula I as defined above, wherein:
X is —S—, —O—, NR^{a} or —CH₂—;
R^{a} is hydrogen or (1-3C)alkyl;
R¹ is selected from hydrogen or a group of the formula:

—X¹-Q¹
wherein
X¹ is —(CH₂)ₙ— or —CO—;
n is 0, 1, 2 or 3;
Q¹ is selected from (1-5C)alkyl, (3-8C)cycloalkyl, aryl, a carbon-linked heterocyclyl, a carbon-linked heteroaryl or —NR⁷R⁸ where R⁷ and R⁸ are each independently selected from methyl or ethyl, or R⁷ and R⁸ are linked so that, together with the nitrogen atom to which they are attached, they form a 4-7 membered heterocyclic ring optionally comprising one, two or three additional heteroatoms selected from N, O or S;
and wherein Q¹ is optionally substituted by one or more substituents selected from halo, trifluoromethyl, trifluoromethoxy, cyano, isocyano, nitro, hydroxy, mercapto, amino, formyl, carboxy, carbamoyl, ureido, (1-4C)alkyl, (2-4C)alkenyl, (2-4C)alkynyl, (1-4C)alkoxy, (1-4C)alkylthio, (1-4C)alkylsulphinyl, (1-4C)alkylsulphonyl, (1-4C)alkylamino, di-[(1-4C)alkyl]amino, (1-4C)alkoxycarbonyl, N-(1-4C)alkylcarbamoyl, N,N-di-[(1-4C)alkyl]carbamoyl, (2-4C)alkanoyl, (2-4C)alkanoyloxy, (2-4C)alkanoylamino, sulphamoyl, N-(1-4C)alkylsulphamoyl, N,N-di-[(1-4C)alkyl]sulphamoyl;
R² is selected from one of the following options:
   (i) when R¹ is hydrogen, R² is selected from (3-5C)alkyl, (3-5C)alkenyl, (3-5C)alkynyl, aryl, (3-8C)cycloalkyl, thienyl or a group of the formula:

—CH₂-Q²
wherein Q² is selected from:
(3-8C)cycloalkyl which is optionally substituted with cyano, nitro, fluoro or methyl;
phenyl which is substituted in the ortho position (relative to the point of attachment to the —CH₂— group) by cyano, nitro or methyl and optionally further substituted in the other ortho position and/or the para position with cyano, nitro, fluoro or methyl;
naphthyl which is optionally substituted with cyano, nitro, fluoro or methyl; or
thiophene which is optionally substituted with cyano, nitro, fluoro or methyl;
(ii) when R¹ is a substituent group other than hydrogen, R² is selected from (3-5C)alkyl, (3-5C)alkenyl, (3-5C)alkynyl, aryl, (3-8C)cycloalkyl, thienyl or a group of the formula:

—CH₂-Q³
wherein Q³ is selected from phenyl, (3-8C)cycloalkyl, naphthyl or a neutral heteroaryl, each of which is optionally substituted by cyano, nitro, halo, methyl, trifluoromethyl, trifluoromethoxy, isocyano, hydroxy, mercapto, amino, carboxy, carbamoyl, methoxy, methylthio, methylsulphinyl, methylsulphonyl, methylamino, or di-methylamino;
R³, R⁴, R⁵ or R⁶ are each independently selected from hydrogen, halo, (1-5C)alkyl or aryl;
with the proviso that R⁴, R⁵ or R⁶ are all hydrogen when R¹ is hydrogen;
or a pharmaceutically acceptable salt or solvate thereof.

Particular novel compounds of the invention include, for example, compounds of the formula I, or pharmaceutically acceptable salts thereof, wherein, unless otherwise stated, each of X, R¹, R², R³, R⁴, R⁵ or R⁶ has any of the meanings defined hereinbefore or in any of paragraphs (1) to (37) hereinafter:—

### (1) X is —S—, —SO— or —O—;

### (2) X is —S— or —SO—;

### (3) X is —S—;

### (4) X is —SO—;

### (5) X is —O—;

### (6) X is —CH₂—;

(7) R¹ is selected from hydrogen or a group of the formula:

X⁰—X¹-Q¹

wherein
X⁰ is absent or —(CH₂)ₙ—;
X¹ is absent, —CO— or —SO₂—;
n is 1 or 2;
when X¹ is absent or —CO— then Q¹ is selected from (1-5C)alkyl, (3-8C)cycloalkyl, phenyl, a carbon-linked heterocyclyl, a carbon-linked heteroaryl or —NR⁷R⁸ where R⁷ and R⁸ are each independently selected from methyl or ethyl, or R⁷ and R⁸ are linked so that, together with the nitrogen atom to which they are attached, they form a 4-6 membered heterocyclic ring optionally comprising one or two additional heteroatoms selected from N, O or S;
when X¹ is —SO₂— then Q¹ is selected from (1-5C)alkyl, (3-8C)cycloalkyl, phenyl or —NR⁷R⁸ (where R⁷ and R⁸ are each as defined above);
and wherein Q¹ is optionally substituted by one or more substituents selected from halo, trifluoromethyl, trifluoromethoxy, cyano, nitro, hydroxy, amino, carboxy, carbamoyl, (1-4C)alkyl, (2-4C)alkenyl, (2-4C)alkynyl, (1-4C)alkoxy, (1-4C)alkylthio, (1-4C)alkylsulphinyl, (1-4C)alkylsulphonyl, (1-4C)alkylamino, di-[(1-4C)alkyl]amino, (1-4C)alkoxycarbonyl, N-(1-4C)alkylcarbamoyl, N,N-di-[(1-4C)alkyl]carbamoyl, (2-4C)alkanoyl, (2-4C)alkanoyloxy, sulphamoyl, N-(1-4C)alkylsulphamoyl, N,N-di-[(1-4C)alkyl]sulphamoyl or a phenyl ring;
(8) R¹ is selected from hydrogen or a group of the formula:

X⁰—X¹-Q¹

wherein
X⁰ is absent or —(CH₂)ₙ—;
X¹ is absent, —CO— or —SO₂—;
n is 1 or 2;
when X¹ is absent or —CO— then Q¹ is selected from (1-5C)alkyl, (3-8C)cycloalkyl, phenyl, a carbon-linked heteroaryl or —NR⁷R⁸ wherein R⁷ and R⁸ are linked so that, together with the nitrogen atom to which they are attached, they form a 4-6 membered heterocyclic ring optionally comprising one additional heteroatom selected from N, O or S;
when X¹ is —SO₂— then Q¹ is selected from (1-5C)alkyl, (3-8C)cycloalkyl, phenyl or —NR⁷R⁸ (where R⁷ and R⁸ are each as defined above);
and wherein Q¹ is optionally substituted by one or more substituents selected from halo, trifluoromethyl, trifluoromethoxy, cyano, nitro, hydroxy, amino, (1-4C)alkyl, (1-4C)alkoxy, (1-4C)alkylthio, (1-4C)alkylsulphinyl, (1-4C)alkylsulphonyl, (1-4C)alkylamino, di-[(1-4C)alkyl]amino, (1-4C)alkoxycarbonyl, (2-4C)alkanoyl, or a phenyl ring;
   (9) R¹ is selected from hydrogen or a group of the formula:

X⁰—X¹-Q¹

wherein
X⁰ is absent or —(CH₂)ₙ—;
X¹ is absent, —CO— or —SO₂—;
n is 1, or 2;
when X¹ is absent or —CO— then Q¹ is selected from (1-5C)alkyl, (3-8C)cycloalkyl, phenyl, a carbon-linked heteroaryl or —NR⁷R⁸ wherein R⁷ and R⁸ are linked so that, together with the nitrogen atom to which they are attached, they form a 4-6 membered heterocyclic ring optionally comprising one additional heteroatom selected from N, O or S;
when X¹ is —SO₂— then Q¹ is selected from (1-5C)alkyl, (3-8C)cycloalkyl, phenyl or —NR⁷R⁸ (where R⁷ and R⁸ are each as defined above);
and wherein Q¹ is optionally substituted by one or more substituents selected from halo, trifluoromethyl, trifluoromethoxy, cyano, nitro, hydroxy, amino, (1-4C)alkyl, (1-4C)alkoxy, (1-4C)alkylthio, (1-4C)alkylsulphinyl, (1-4C)alkylsulphonyl, (1-4C)alkylamino, di-[(1-4C)alkyl]amino, (1-4C)alkoxycarbonyl, (2-4C)alkanoyl, or a phenyl ring;
   (10) In the second aspect of the invention, R¹ is selected from hydrogen or a group of the formula:

—X¹-Q¹
wherein
X¹ is —(CH₂)ₙ— or —CO—;
n is 0 or 1;
Q¹ is selected from (1-5C)alkyl, (3-8C)cycloalkyl, aryl, a carbon-linked heterocyclyl, a carbon-linked heteroaryl or —NR⁷R⁸ where R⁷ and R⁸ are each independently selected from methyl or ethyl, or R⁷ and R⁸ are linked so that, together with the nitrogen atom to which they are attached, they form a 4-7 membered heterocyclic ring optionally comprising one, two or three additional heteroatoms selected from N, O or S;
and wherein Q¹ is optionally substituted by one or more substituents selected from halo, trifluoromethyl, trifluoromethoxy, cyano, isocyano, nitro, hydroxy, mercapto, amino, formyl, carboxy, carbamoyl, ureido, (1-4C)alkyl, (2-4C)alkenyl, (2-4C)alkynyl, (1-4C)alkoxy, (1-4C)alkylthio, (1-4C)alkylsulphinyl, (1-4C)alkylsulphonyl, (1-4C)alkylamino, di-[(1-4C)alkyl]amino, (1-4C)alkoxycarbonyl, N-(1-4C)alkylcarbamoyl, N,N-di-[(1-4C)alkyl]carbamoyl, (2-4C)alkanoyl, (2-4C)alkanoyloxy, (2-4C)alkanoylamino, sulphamoyl, N-(1-4C)alkylsulphamoyl, N,N-di-[(1-4C)alkyl]sulphamoyl;
   (11) In the second aspect of the invention, R¹ is selected from hydrogen or a group of the formula:

—X¹-Q¹
wherein
X¹ is —(CH₂)ₙ— or —CO—;
n is 0 or 1;
Q¹ is selected from (1-5C)alkyl, (3-6C)cycloalkyl, aryl, a carbon-linked heteroaryl or —NR⁷R⁸ where R⁷ and R⁸ are each independently selected from methyl, or R⁷ and R⁸ are linked so that, together with the nitrogen atom to which they are attached, they form a 4-6 membered heterocyclic ring optionally comprising one, two or three additional heteroatoms selected from N, O or S;
and wherein Q¹ is optionally substituted by one or more substituents selected from halo, trifluoromethyl, trifluoromethoxy, cyano, isocyano, nitro, hydroxy, mercapto, amino, formyl, carboxy, carbamoyl, ureido, (1-2C)alkyl, (1-2C)alkoxy, (1-2C)alkylthio, (1-2C)alkylsulphinyl, (1-2C)alkylsulphonyl, (1-2C)alkylamino, di-[(1-2C)alkyl]amino, (1-2C)alkoxycarbonyl, N-(1-2C)alkylcarbamoyl, N,N-di-[(1-2C)alkyl]carbamoyl, (2C)alkanoyl, (2C)alkanoyloxy, (2C)alkanoylamino, sulphamoyl, N-(1-2C)alkylsulphamoyl, N,N-di-[(1-2C)alkyl]sulphamoyl;
   (12) In the second aspect of the invention, R¹ is selected from hydrogen or a group of the formula:

—X¹-Q¹
wherein
X¹ is —(CH₂)ₙ— or —CO—;
n is 0 or 1;
Q¹ is selected from (1-5C)alkyl, (3-6C)cycloalkyl, aryl, a carbon-linked heteroaryl;
and wherein Q¹ is optionally substituted by one or more substituents selected from halo, trifluoromethyl, trifluoromethoxy, cyano, isocyano, nitro, hydroxy, mercapto, amino, carboxy, carbamoyl, ureido, (1-2C)alkyl, (1-2C)alkoxy, (1-2C)alkylthio, (1-2C)alkylsulphinyl, (1-2C)alkylsulphonyl, (1-2C)alkylamino, di-[(1-2C)alkyl]amino, (1-2C)alkoxycarbonyl, (2C)alkanoyl, or (2C)alkanoyloxy;
   (13) R² is selected from one of the following options:
(i) when R¹ is hydrogen, R² is selected from (3-5C)alkyl, phenyl, (3-8C)cycloalkyl, thienyl or a group of the formula:

—CH₂-Q²
wherein Q² is selected from:
(3-8C)cycloalkyl which is optionally substituted with cyano, nitro, fluoro or methyl;
phenyl which is optionally substituted in the ortho or para position (relative to the point of attachment to the —CH₂— group) by cyano, nitro, methyl, —CO₂H and tetrazole and optionally further substituted with cyano, nitro, fluoro, or methyl;
thiophene which is optionally substituted with cyano, nitro, fluoro or methyl;
(ii) when R¹ is a substituent group other than hydrogen, R² is selected from (3-5C)alkyl, phenyl, (3-8C)cycloalkyl, thienyl or a group of the formula:

—CH₂-Q³
wherein Q³ is selected from phenyl, (3-8C)cycloalkyl, or a 5 or 6-membered neutral heteroaryl, each of which is optionally substituted by cyano, nitro, halo, methyl, trifluoromethyl, trifluoromethoxy, hydroxy, amino, methoxy, methylthio, methylsulphinyl, methylsulphonyl, methylamino or di-methylamino;
   (14) R² is selected from one of the following options:
(i) when R¹ is hydrogen, R² is selected from (3-5C)alkyl, or a group of the formula:

—CH₂-Q²
wherein Q² is selected from:
phenyl which is optionally substituted in the ortho or para position (relative to the point of attachment to the —CH₂— group) by cyano, nitro, methyl, —CO₂H and tetrazole and optionally further substituted with cyano, nitro, fluoro, or methyl;
(ii) when R¹ is a substituent group other than hydrogen, R² is selected from (3-5C)alkyl or a group of the formula:

—CH₂-Q³
wherein Q³ is selected from phenyl, which is optionally substituted by cyano, nitro, halo, methyl, trifluoromethyl, trifluoromethoxy, hydroxy, amino, methoxy, methylthio, methylsulphinyl, methylsulphonyl, methylamino or di-methylamino;
   (15) R² is selected from one of the following options:
(i) when R¹ is hydrogen, R² is selected from (3-5C)alkyl, or a group of the formula:

—CH₂-Q²
wherein Q² is selected from:
phenyl which is optionally substituted in the ortho position (relative to the point of attachment to the —CH₂— group) by cyano, nitro, methyl, —CO₂H and tetrazole and optionally further substituted with cyano, nitro, fluoro, or methyl;
(ii) when R¹ is a substituent group other than hydrogen, R² is selected from (3-5C)alkyl or a group of the formula:

—CH₂-Q³
wherein Q³ is selected from phenyl, which is optionally substituted by cyano, nitro, halo, methyl, trifluoromethyl, hydroxy, or methoxy;
   (16) R² is selected from one of the following options:
(i) when R¹ is hydrogen, R² is selected from (3-5C)alkyl, phenyl, (3-6C)cycloalkyl, thienyl or a group of the formula:

—CH₂-Q²
wherein Q² is selected from:
phenyl which is substituted in the ortho position (relative to the point of attachment to the —CH₂— group) by cyano, nitro or methyl and optionally further substituted in the other ortho position and/or the para position with cyano, nitro, fluoro or methyl;
naphthyl which is optionally substituted with cyano, nitro, fluoro or methyl; or
thiophene which is optionally substituted with cyano, nitro, fluoro or methyl;
(ii) when R¹ is a substituent group other than hydrogen, R² is selected from (3-5C)alkyl, phenyl, (3-6C)cycloalkyl, thienyl or a group of the formula:

—CH₂-Q³
wherein Q³ is selected from phenyl or a non-basic heteroaryl (e.g. thiophene), each of which is optionally substituted by cyano, nitro, halo, methyl, trifluoromethyl, trifluoromethoxy, isocyano, hydroxy, mercapto, amino, carboxy, carbamoyl, methoxy, methylthio, methylsulphinyl, methylsulphonyl, methylamino, or di-methylamino;
   (17) R² is selected from one of the following options:
(i) when R¹ is hydrogen, R² is selected from (3-5C)alkyl or a group of the formula:

—CH₂-Q²
wherein Q² is phenyl which is substituted in the ortho position (relative to the point of attachment to the —CH₂— group) by cyano, nitro or methyl and optionally further substituted in the other ortho position and/or the para position with cyano, nitro, fluoro or methyl;
(ii) when R¹ is a substituent group other than hydrogen, R² is selected from (3-5C)alkyl or a group of the formula:

—CH₂-Q³
wherein Q³ is phenyl optionally substituted by cyano, nitro, halo, or methyl;
   (18) R² is selected from one of the following options:
(i) when R¹ is hydrogen, R² is selected from (3-5C)alkyl or a group of the formula:

—CH₂-Q²
wherein Q² is phenyl which is substituted in the ortho position (relative to the point of attachment to the —CH₂— group) by cyano, nitro or methyl and optionally further substituted in the other ortho position and/or the para position with cyano, nitro, fluoro or methyl;
(ii) when R¹ is a substituent group other than hydrogen, R² is selected from (3-5C)alkyl or a group of the formula:

—CH₂-Q³
wherein Q³ is phenyl which is substituted in the ortho position (relative to the point of attachment to the —CH₂— group) by cyano, nitro or methyl and optionally further substituted in the other ortho position and/or the para position with cyano, nitro, fluoro or methyl;
   (19) R² is selected from (3-5C)alkyl or a group of the formula:

—CH₂-Q²
wherein Q² is phenyl which is substituted in the ortho position (relative to the point of attachment to the —CH₂— group) by cyano, nitro or methyl and optionally further substituted in the other ortho position and/or the para position with cyano, nitro, fluoro or methyl;
   (20) R² is a group of the formula:

—CH₂-Q²
wherein Q² is phenyl which is substituted in the ortho position (relative to the point of attachment to the —CH₂— group) by cyano, nitro or methyl;
   (21) R² is a group of the formula:

—CH₂-Q²
wherein Q² is phenyl which is substituted in the ortho position (relative to the point of attachment to the —CH₂— group) by cyano;
   (22) R³ is selected from hydrogen, halo, (1-5C)alkyl, (3-6C)cycloalkyl, (3-6C)cycloalkyl(1-2C)alkyl, thiophene or phenyl, wherein any phenyl ring is optionally substituted by halo, methoxy, ethoxy, methyl or ethyl;
   (23) R³ is selected from hydrogen, halo, (1-3C)alkyl, (3-6C)cycloalkyl, (3-6C)cycloalkyl(1-2C)alkyl, thiophene or phenyl, wherein any phenyl ring is optionally substituted by halo, methoxy, methyl;
   (24) R³ is selected from hydrogen, halo, (1-5C)alkyl or phenyl;
   (25) R³ is selected from hydrogen, halo, (1-3C)alkyl or phenyl;
   (26) R³ is selected from hydrogen or halo;
   (27) R³ is selected from hydrogen, fluoro, chloro or bromo;
   (28) R³ is hydrogen;
   (29) R⁴ is hydrogen when R¹ is hydrogen or selected from hydrogen, halo, (1-5C)alkyl or phenyl when R¹ is a substituent other than hydrogen;
   (30) R⁴ is hydrogen when R¹ is hydrogen or selected from hydrogen or halo when R¹ is a substituent other than hydrogen;
   (31) R⁴ is hydrogen;
   (32) R⁵ is hydrogen when R¹ is hydrogen or selected from hydrogen, halo, (1-5C)alkyl or phenyl when R¹ is a substituent other than hydrogen;
   (33) R⁵ is hydrogen when R¹ is hydrogen or selected from hydrogen or halo when R¹ is a substituent other than hydrogen;
   (34) R⁵ is hydrogen;
   (35) R⁶ is hydrogen when R¹ is hydrogen or selected from hydrogen, halo, (1-5C)alkyl or phenyl when R¹ is a substituent other than hydrogen;
   (36) R⁶ is hydrogen when R¹ is hydrogen or selected from hydrogen or halo when R¹ is a substituent other than hydrogen; or
   (37) R⁶ is hydrogen.

Suitably, X¹ is as defined in any one of paragraphs (1) to (5) above.

Suitably, R¹ is as defined in any one of paragraphs (7) to (9) above.

Suitably, X⁰ is absent or —CH₂—.

In an embodiment, X⁰ and X¹ are both absent. In another embodiment, at least one of X⁰ and X¹ is a substituent group (i.e. at least one of X⁰ and X¹ is not absent).

Suitably, Q¹ is as defined in any one of paragraphs (7) to (9) above.

Suitably, R² is as defined in any one of paragraphs (13) to (21) above.

Suitably, Q¹ and Q² have any one of the definitions set out in any one of paragraphs (13) to (21) above.

Suitably, R³ is as defined in any one of paragraphs (22) to (28) above.

Suitably, R⁴, R⁵ and R⁶ are all hydrogen.

In a particular group of compounds of the invention, X is S or —SO—; R² is a group —CH₂-Q² or —CH₂Q³ as defined herein; and R¹ is substituent other than hydrogen, i.e. a group of the formula:

X⁰—X-Q¹

wherein

X⁰ is absent or —(CH₂)ₙ—;

X¹ is absent, —CO— or —SO₂—;

n is 1 or 2;

when X¹ is absent then Q¹ is selected from (3-8C)cycloalkyl, aryl, a carbon-linked heterocyclyl, a carbon-linked heteroaryl or —NR⁷R⁸ where R⁷ and R⁸ are each independently selected from methyl or ethyl, or R⁷ and R⁸ are linked so that, together with the nitrogen atom to which they are attached, they form a 4-7 membered heterocyclic ring optionally comprising one, two or three additional heteroatoms selected from N, O or S;

when X¹ is —CO— then Q¹ is selected from (1-5C)alkyl, (3-8C)cycloalkyl, aryl, a carbon-linked heterocyclyl, a carbon-linked heteroaryl or —NR⁷R⁸ where R⁷ and R⁸ are each independently selected from methyl or ethyl, or R⁷ and R⁸ are linked so that, together with the nitrogen atom to which they are attached, they form a 4-7 membered heterocyclic ring optionally comprising one, two or three additional heteroatoms selected from N, O or S;

when X¹ is —SO₂— then Q¹ is selected from (1-5C)alkyl, (3-8C)cycloalkyl, phenyl, thiophene or —NR⁷R⁸;

and wherein Q¹ is optionally substituted by one or more substituents selected from halo, trifluoromethyl, trifluoromethoxy, cyano, isocyano, nitro, hydroxy, mercapto, amino, formyl, carboxy, carbamoyl, ureido, (1-4C)alkyl, (2-4C)alkenyl, (2-4C)alkynyl, (1-4C)alkoxy, (1-4C)alkylthio, (1-4C)alkylsulphinyl, (1-4C)alkylsulphonyl, (1-4C)alkylamino, di-[(1-4C)alkyl]amino, (1-4C)alkoxycarbonyl, N-(1-4C)alkylcarbamoyl, N,N-di-[(1-4C)alkyl]carbamoyl, (2-4C)alkanoyl, (2-4C)alkanoyloxy, (2-4C)alkanoylamino, sulphamoyl, N-(1-4C)alkylsulphamoyl, N,N-di-[(1-4C)alkyl]sulphamoyl or a phenyl ring which is optionally further substituted by halo, methoxy, ethoxy, methyl, ethyl, cyano or hydroxy.

In a group of compounds of the invention, X is S or —SO—; R² is a group —CH₂-Q² or —CH₂Q³ as defined herein; and R¹ may be a substituent other than hydrogen as defined in any one of paragraphs (7) to (9) above with the proviso that if X¹ is absent then Q¹ cannot be (1-3C)alkyl.

In a further group of compounds, X is S or —SO—; R² is a group —CH₂-Q² or —CH₂Q³ as defined herein; R¹ may be a substituent other than hydrogen as defined in any one of paragraphs (7) to (9) above with the proviso that if X¹ is absent then Q¹ cannot be (1-5C)alkyl.

In a particular group of compounds of the invention, R⁴, R⁵ and R⁶ are all hydrogen. Such compounds have the structural formula II shown below

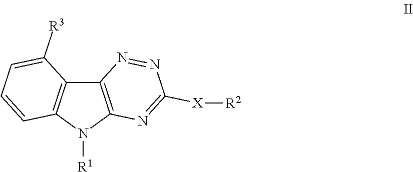

wherein X, R¹, R² and R³ each have any one of the definitions set out hereinbefore.

In an embodiment, X in the compounds of formula II is —S—, —SO— or —O—, especially —S— or —SO—.

In another embodiment, R¹ is as defined above in any one of paragraphs (7) to (12) in the compounds of formula II.

In another embodiment, R² is as defined above in any one of paragraphs (13) to (21) in the compounds of formula II.

In another embodiment, R³ is as defined above in any one of paragraphs (22) to (28) in the compounds of formula II.

In a further group of compounds of the invention, R³, R⁴, R⁵ and R⁶ are all hydrogen. Such compounds have the structural formula III shown below

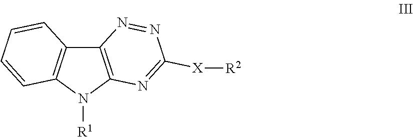

wherein X, R¹ and R² each have any one of the definitions set out hereinbefore.

In an embodiment, X in the compounds of formula III is —S— or —O—, especially —S—.

In another embodiment, R¹ is as defined above in any one of paragraphs (7) to (12) in the compounds of formula III.

In another embodiment, R² is as defined above in any one of paragraphs (13) to (21) in the compounds of formula III.

In another embodiment, R³ is as defined above in any one of paragraphs (22) to (28) in the compounds of formula III.

In a further group of compounds of the invention, R² is as defined in any one or paragraphs (13), (14), (15), (16) (17), (18), (19), (20) or (21) above.

Particular compounds of the invention include any one of the following:
3-((2′-nitrobenzyl)thio)-5H-[1,2,4]triazino[5,6-b]indole;
3-((2′-cyanobenzyl)thio)-5H-[1,2,4]triazino[5,6-b]indole;
2′-(((5-benzyl-5H-[1,2,4]triazino[5,6-b]indol-3-yl)thio)methyl)benzonitrile;
5-benzyl-3-(2′-fluorobenzylthio)-5H-[1,2,4]triazino[5,6-b]indole;
2′(((5-(4″-chlorobenzyl)-5H-[1,2,4]triazino[5,6-b]indol-3-yl)thio)methyl)benzonitrile;
5-(4″-chlorobenzyl)-3-((2′-nitrobenzyl)thio)-5H-[1,2,4]triazino[5,6-b]indole;
(3-((2′-nitrobenzyl)thio)-5H-[1,2,4]triazino[5,6-b]indol-5-yl)(phenyl)methanone;
2′-(((9-bromo-5H-[1,2,4]triazino[5,6-b]indol-3-yl)thio)methyl)benzonitrile;
9-bromo-3-((2′-nitrobenzyl)thio)-5H-[1,2,4]triazino[5,6-b]indole;
2′-(((5-(2″-nitrobenzyl)-5H-[1,2,4]triazino[5,6-b]indol-3-yl)thio)methyl)benzonitrile;
3-((2′,4′-dinitrobenzyl)thio)-5H-[1,2,4]triazino[5,6-b]indole;
3-((2′-fluoro-6′-nitrobenzyl)thio)-5H-[1,2,4]triazino[5,6-b]indole;
5-(2″-fluorobenzyl)-3-((2′-nitrobenzyl)thio)-5H-[1,2,4]triazino[5,6-b]indole;
5-benzyl-3-((2′-nitrobenzyl)thio)-5H-[1,2,4]triazino[5,6-b]indole;
3-((2′-methylbenzyl)thio)-5H-[1,2,4]triazino[5,6-b]indole;
2′-(((5-(4″-dimethylamino)benzoyl)-5H-[1,2,4]triazino[5,6-b]indol-3-yl)thio)methyl)benzonitrile;
2′-(((5-(3″,4″-dimethoxybenzoyl)-5H-[1,2,4]triazino[5,6-b]indol-3-yl)thio)methyl)benzonitrile;
2′-(((5-(cyclohexanecarbonyl)-5H-[1,2,4]triazino[5,6-b]indol-3-yl)thio)methyl)benzonitrile;
2′-(((5-(3″-trifluoromethyl)benzoyl)-5H-[1,2,4]triazino[5,6-b]indol-3-yl)thio)methyl)benzonitrile;
2′-(((5-(4″-fluorobenzoyl)-5H-[1,2,4]triazino[5,6-b]indol-3-yl)thio)methyl)benzonitrile;
2′-(((5-(4″-(trifluoromethoxy)benzoyl)-5H-[1,2,4]triazino[5,6-b]indol-3-yl)thio)methyl)benzonitrile;
2′-(((5-(cyclopentanecarbonyl)-5H-[1,2,4]triazino[5,6-b]indol-3-yl)thio)methyl)benzonitrile;
2′-(((5-(furan-2-carbonyl)-5H-[1,2,4]triazino[5,6-b]indol-3-yl)thio)methyl)benzonitrile;
2′-(((5-(isoxazole-5″-carbonyl)-5H-[1,2,4]triazino[5,6-b]indol-3-yl)thio)methyl)benzonitrile;
2′-(((5-(benzoyl-5H-[1,2,4]triazino[5,6-b]indol-3-yl)thio)methyl)benzonitrile;
methyl 4′-(3-((2′-cyanobenzyl)thio)-5H-[1,2,4]triazino[5,6-b]indol-5-yl)-4′-oxobutanoate;
methyl 5′-(3-((2′-cyanobenzyl)thio)-5H-[1,2,4]triazino[5,6-b]indol-5-yl)-5′-oxopentanoate;
3-(butylthio)-5H-[1,2,4]triazino[5,6-b]indole;
2′(((5-acetyl-5H-[1,2,4]triazino[5,6-b]indol-3-yl)thio)methyl)benzonitrile;
2′-(((5-ethyl-5H-[1,2,4]triazino[5,6-b]indol-3-yl)thio)methyl)benzonitrile;
2′-(((5-butyl-5H-[1,2,4]triazino[5,6-b]indol-3-yl)thio)methyl)benzonitrile;
2′-(((5H-[1,2,4]triazino[5,6-b]indol-3-yl)sulfinyl)methyl)benzonitrile;
3-((4′-Nitrobenzyl)thio)-5H-[1,2,4]triazino[5,6-b]indole;
2′-(((5-(phenylsulfonyl)-5H-[1,2,4]triazino[5,6-b]indol-3-yl)thio)methyl)benzonitrile;
2′-(((5-(morpholine-4′-carbonyl)-5H-[1,2,4]triazino[5,6-b]indol-3-yl)thio)methyl)benzonitrile;
2′-(((5-(cyclohexylmethyl)-5H-[1,2,4]triazino[5,6-b]indol-3-yl)thio)methyl)benzonitrile;
2′-(((5-(4′-fluorobenzyl)-5H-[1,2,4]triazino[5,6-b]indol-3-yl)thio)methyl)benzonitrile;
2′-((5-(2″-phenylcyclopropanecarboonyl)-5H-[1,2,4]triazino[5,6-b]indol-3-ylthio)methyl)benzonitrile;
2′-((5-(2-Cyclopropyl-2-oxoethyl)-5H-[1,2,4]triazino[5,6-b]indol-3-ylthio)methyl)benzonitrile;
2′-((5-(2″-methylcyclopropanecarbonyl)-5H-[1,2,4]triazino[5,6-b]indol-3-ylthio)methyl)benzonitrile;
2′-((5-(Cyclopropylmethyl)-5H-[1,2,4]triazino[5,6-b]indol-3-ylthio)methyl)benzonitrile;
2′-(((5-(Cyclopropanecarbonyl)-5H-[1,2,4]triazino[5,6-b]indole-3-yl)thio)methyl)benzonitrile;
2′-(((9-Bromo-5-(cyclopropylmethyl)-5H-[1,2,4]triazino[5,6-b]indol-3-yl)thio)methyl)benzonitrile;
2′-(((9-Bromo-5-(cyclopropanecarbonyl)-5H-[1,2,4]triazino[5,6-b]indol-3-yl)thio)methyl)benzonitrile;
Methyl-4′-(9-bromo-3-((2′-cyanobenzyl)thio)-5H-[1,2,4]triazino[5,6-b]indol-5-yl)-4′-oxobutyrate;
2′-(((5H-[1,2,4]triazino[5,6-b]indol-3-yl)thio)methyl)benzoic acid;
3-(((2-(2H-tetrazol-5-yl)benzyl)thio)-5H-[1,2,4]triazino[5,6-b]indole;
2″-(((9-(o-Tolyl)-5H-[1,2,4]triazino[5,6-b]indol-3-yl)thio)methyl)benzonitrile;
2″-(((9-(4′-Methoxyphenyl)-5H-[1,2,4]triazino[5,6-b]indol-3-yl)thio)methyl)benzonitrile;
2″-(((9-(Thiophen-3′-yl)-5H-[1,2,4]triazino[5,6-b]indol-3-yl)thio)methyl)benzonitrile;
2″-(((9-cyclopropyl-5H-[1,2,4]triazino[5,6-b]indol-3-yl)thio)methyl)benzonitrile;
   or a pharmaceutically acceptable salt or solvate thereof.

In a particular embodiment, the compounds of formula I are subject to any one of the following provisos:
(i) when R³, R⁴, R⁵ or R⁶ are hydrogen, X is —S— and R¹ is a group —X¹-Q¹ in which X¹ is —CO— and Q¹ is methyl, then R² is not a group —CH₂-Q³ in which Q³ is a 2-nitrophenyl group;
(ii) when R³, R⁴, R⁵ or R⁶ are hydrogen, X is —S— and R¹ is a group —X¹-Q¹ in which X¹ is —CH₂— and Q¹ is 2-cyanophenyl, then R² is not a group —CH₂-Q³ in which Q³ is a 2-cyanophenyl group;
(iii) when R³, R⁴, R⁵ or R⁶ are hydrogen, X is —S— and R¹ is a group —X¹-Q¹ in which X¹ is —CO— and Q¹ is 4-fluoro-2-trifluoromethylphenyl, then R² is not a group —CH₂-Q³ in which Q³ is a 2-cyanophenyl group.

In a further embodiment, when R¹ is an alkanoyl group (i.e. a group —X¹-Q¹ in which X¹ is —CO—, Q¹ is (1-5C)alkyl) then it suitably comprises three or more carbon atoms (i.e. Q¹ is selected from (2-5C)alkyl.

In a further embodiment, R¹ is selected from hydrogen or a group —X¹-Q¹ that comprises three or more carbon atoms.

The various functional groups and substituents making up the compounds of the invention are typically chosen such that the molecular weight of the compound of the invention does not exceed 1000. More usually, the molecular weight of the compound will be less than 750, for example less than 700, or less than 650, or less than 600, or less than 550. More preferably, the molecular weight is less than 525 and, for example, is 500 or less.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess CB2R agonist activity.

The present invention also encompasses compounds of the invention as defined herein which comprise one or more isotopic substitutions. For example, H may be in any isotopic form, including ¹H, ²H(D), and ³H (T); C may be in any isotopic form, including ¹²C, ¹³C, and ¹⁴C; and O may be in any isotopic form, including ¹⁶0 and ¹⁸0; and the like.

It is also to be understood that certain compounds of the invention may exist in solvated as well as unsolvated forms such as, for example, hydrated forms. It is to be understood that the invention encompasses all such solvated forms that possess CB2R agonist activity.

It is also to be understood that certain compounds of the invention may exhibit polymorphism, and that the invention encompasses all such forms that possess CB2R agonist activity.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro. In addition, certain heterocycles may also exist in different tautomeric forms.

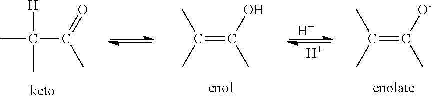

Compounds of the invention containing an amine function may also form N-oxides. A reference herein to a compound of the invention that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

The compounds of the invention may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the invention and in-vivo cleavable amide derivatives that may be formed at a carboxy group or an amino group in a compound of the invention.

Accordingly, the present invention includes those compounds of the invention as defined hereinbefore when made available by organic synthesis and when made available within the human or animal body by way of cleavage of a pro-drug thereof. Accordingly, the present invention includes those compounds of the invention that are produced by organic synthetic means and also such compounds that are produced in the human or animal body by way of metabolism of a precursor compound, that is a compound of the invention may be a synthetically-produced compound or a metabolically-produced compound.

A suitable pharmaceutically acceptable pro-drug of a compound of the invention is one that is based on reasonable medical judgement as being suitable for administration to the human or animal body without undesirable pharmacological activities and without undue toxicity.

Various forms of pro-drug have been described, for example in the following documents:—

a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);

### d) H. Bundgaard, Advanced Drug Delivery Reviews, 8, 1-38 (1992);

### e) H. Bundgaard, et al., Journal of Pharmaceutical Sciences, 77, 285 (1988);

f) N. Kakeya, et al., Chem. Pharm. Bull., 32, 692 (1984);

### g) T. Higuchi and V. Stella, “Pro-Drugs as Novel Delivery Systems”, A.C.S. Symposium Series, Volume 14; and

h) E. Roche (editor), “Bioreversible Carriers in Drug Design”, Pergamon Press, 1987.

A suitable pharmaceutically acceptable pro-drug of a compound of the invention that possesses a carboxy group is, for example, an in vivo cleavable ester thereof. An in vivo cleavable ester of a compound of the invention containing a carboxy group is, for example, a pharmaceutically acceptable ester which is cleaved in the human or animal body to produce the parent acid. Suitable pharmaceutically acceptable esters for carboxy include C₁₋₆alkyl esters such as methyl, ethyl and tert-butyl, C₁₋₆alkoxymethyl esters such as methoxymethyl esters, C₁₋₆alkanoyloxymethyl esters such as pivaloyloxymethyl esters, 3-phthalidyl esters, C₃₋₈cycloalkylcarbonyloxy-C₁₋₆alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆alkoxycarbonyloxy-C₁₋₆alkyl esters such as methoxycarbonyloxymethyl and 1-methoxycarbonyloxyethyl esters.

A suitable pharmaceutically acceptable pro-drug of a compound of the invention that possesses a hydroxy group is, for example, an in vivo cleavable ester or ether thereof. An in vivo cleavable ester or ether of a compound of the invention containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁₋₁₀alkanoyl groups such as acetyl, benzoyl, phenylacetyl and substituted benzoyl and phenylacetyl groups, C₁₋₁₀alkoxycarbonyl groups such as ethoxycarbonyl, N,N—(C₁₋₆)₂-carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the invention that possesses a carboxy group is, for example, an in vivo cleavable amide thereof, for example an amide formed with an amine such as ammonia, a C₁₋₄alkylamine such as methylamine, a (C₁₋₄alkyl)₂-amine such as dimethylamine, N-ethyl-N-methylamine or diethylamine, a C₁₋₄alkoxy-C₂₋₄alkylamine such as 2-methoxyethylamine, a phenyl-C₁₋₄alkylamine such as benzylamine and amino acids such as glycine or an ester thereof.

A suitable pharmaceutically acceptable pro-drug of a compound of the invention that possesses an amino group is, for example, an in vivo cleavable amide derivative thereof. Suitable pharmaceutically acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl.

The in vivo effects of a compound of the invention may be exerted in part by one or more metabolites that are formed within the human or animal body after administration of a compound of the invention. As stated hereinbefore, the in vivo effects of a compound of the invention may also be exerted by way of metabolism of a precursor compound (a pro-drug).

### Synthesis

The compounds of the present invention can be prepared by any suitable technique known in the art.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a compound of formula I will vary depending on the nature of the substituent groups. Suitable processes for the preparation of these compounds are described further in the accompanying examples (see Examples 1-29).

In a particular aspect, the present invention provides a method of synthesising a compound of formula I (in which X is S or O) as defined herein, said method comprising:
(i) the reaction of a compound of formula IA:

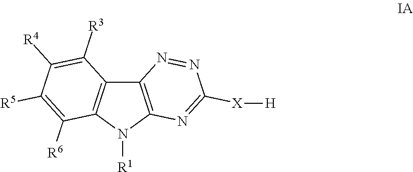

wherein X is S or O and R¹, R³, R⁴, R⁵ and R⁶ are each as defined hereinbefore; with a compound of formula IB:

R²-L IB
wherein R² is as defined hereinbefore and L is a leaving group;
in a suitable solvent.

Suitably, the reaction is carried out in the presence of a suitable base, such as triethylamine.

Any suitable solvent may be used to facilitate this reaction. In an embodiment, the solvent is a lower alcohol, for example methanol.

In a further aspect, the present invention provides a method of synthesising a compound of formula I in which X is SO as defined herein, said method comprising synthesising a compound of formula I in which X is S as defined above and oxidising the S group to SO with a suitable reducing agent (such as a peracid or MPCBA) in a suitable solvent.

In a further aspect, the present invention provides a method of synthesising a compound of formula I as defined herein, said method comprising:
(i) the reaction of a compound of formula IC:

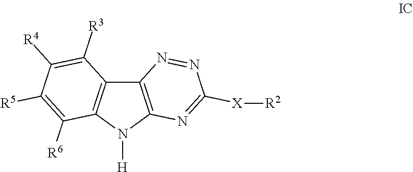

wherein X, and R², R³, R⁴, R⁵ and R⁶ are each as defined hereinbefore;
with a compound of formula ID:

R¹-L ID
wherein R¹ is as defined hereinbefore and L is a suitable leaving group;
in a suitable solvent.

Suitably, this reaction is carried out in the presence of an inert atmosphere, such as argon.

Any suitable solvent may also be used to facilitate this reaction. In an embodiment, the solvent is an amidic solvent such as dimethyl formamide or an ethereal solvent such as THF.

Suitable leaving groups L for compounds IB or ID are well known in the art. In a particular embodiment, L is a halogen, such chloride, bromide, or iodide.

Once a compound of formula I has been synthesised by any one of the methods defined herein, the method may then further comprise the additional steps of:
(i) removing any protecting groups present;
(ii) converting the compound formula I into another compound of formula I; and/or
(iii) forming a pharmaceutically acceptable salt or solvate thereof.

An example of (ii) above is when a compound of formula I is synthesised and then one or more of the groups R¹ to R⁶ may be further reacted to change the nature of the group and thereby provide an alternative compound of formula I.

The resultant compounds of formula I can be isolated and purified using techniques well known in the art.

In a further aspect of the invention, there is provided a compound of formula I obtainable by a process as defined herein.

In a further aspect of the invention, there is provided a compound of formula I obtained by a process as defined herein.

In a further aspect of the invention, there is provided a compound of formula I directly obtained by a process as defined herein.

### Biological Activity

The biological assays defined herein (Assay 1 and Assay 2 described in Example 30) may be used to measure the pharmacological effects of the compounds of the present invention.

Although the pharmacological properties of the compounds of the invention vary with structural change, as expected, the compounds of the invention were found to demonstrate agonist activity in these biological assays.

In general, the compounds of the invention demonstrate IC₅₀ values in Assays 1 and 2 described in Example 52 of less than 5 μM. Preferred compounds demonstrate IC₅₀ values of less than 2 μM. More preferred compounds demonstrate IC₅₀ values of less than 1 μM. Most preferred compounds demonstrate IC₅₀ values of less than 500 nM.

### Pharmaceutical Compositions

According to a further aspect of the invention there is provided a pharmaceutical composition which comprises a compound of the invention as defined hereinbefore, or a pharmaceutically acceptable salt or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the invention will naturally vary according to the nature and severity of the conditions, the age and sex of the animal or patient and the route of administration, according to well known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The compounds of the invention demonstrate CB2R agonist activity and, accordingly, are potentially useful for the treatment of diseases or conditions in which the stimulation of CB2R is beneficial.

In one aspect, the present invention provides a compound of formula I as defined herein, or a pharmaceutically acceptable salt or solvate thereof, for use in therapy.

In another aspect the present invention provides a method of treating a condition in which the stimulation of CB2R is beneficial (as defined herein), the method comprising administering a therapeutically effective amount of a compound of formula I as defined herein, or a pharmaceutically acceptable salt or solvate thereof, to a subject in need of such treatment.

The subject may be a human or animal patient.

In another aspect, the present invention provides a compound of formula I as defined herein, or a pharmaceutically acceptable salt or solvate thereof, for use in the treatment of a condition in which the stimulation of CB2R is beneficial (as defined herein).

In another aspect, the present invention provides the use of a compound of formula I as defined herein, or a pharmaceutically acceptable salt or solvate thereof, in the manufacture of a medicament for use in the treatment of a condition in which the stimulation of CB2R is beneficial (as defined herein).

In particular, the compounds of the present invention are anti-inflammatory agents suitable for use in the treatment of inflammation and conditions associated therewith.

Thus, in a particular aspect, the present invention provides a method of treating an inflammatory condition, the method comprising administering a therapeutically effective amount of a compound of formula I as defined herein, or a pharmaceutically acceptable salt or solvate thereof, to a subject in need of such treatment.

In another particular aspect, the present invention provides a compound of formula I as defined herein, or a pharmaceutically acceptable salt or solvate thereof, for use in the treatment of inflammation.

In another aspect, the present invention provides the use of a compound of formula I as defined herein, or a pharmaceutically acceptable salt or solvate thereof, in the manufacture of a medicament for use in the treatment of inflammation.

Particular examples of diseases or conditions that could be treated with such agents include cardiovascular disease (e.g. atherosclerosis), inflammatory pain (both peripheral inflammatory pain and chronic inflammatory pain), allergies and physical wounds.

In another aspect, the present invention provides a compound of formula I as defined herein, or a pharmaceutically acceptable salt or solvate thereof, for use as a CB2R agonist (in vitro or in vivo).

In another aspect, the present invention provides the use of a compound of formula I as defined herein, or a pharmaceutically acceptable salt or solvate thereof, in the manufacture of a medicament for use as an agonist of CB2R.

In another aspect, the present invention a method of stimulating CB2R in a cell, tissue or a subject, the method comprising administering a compound of formula I as defined herein, or a pharmaceutically acceptable salt or solvate thereof to said cell, tissue or subject.

### Route of Administration

The compounds of the invention or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); perenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The CB2R agonists of the present invention may be used as a sole therapy or may involve, in addition to the compound of the invention, therapy with one or more additional therapeutic agents.

Thus, in another aspect, the present invention provides a compound of formula I as defined herein, or a pharmaceutically acceptable salt or solvate thereof, for use in the treatment of a condition in which the stimulation of CB2R is beneficial (as defined herein) in combination with one or more additional therapeutic agents.

In another aspect, the present invention provides the use of a compound of formula I as defined herein, or a pharmaceutically acceptable salt or solvate thereof, in the manufacture of medicament for use in the treatment of a condition in which the stimulation of CB2R is beneficial (as defined herein) in combination with one or more additional therapeutic agents.

The other therapeutic agents that may be used may include one or more additional anti-inflammatory agents (such as, for example, NSAIDs, COX-2 inhibitors or steroids) or one or more analgesics.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to a further aspect of the invention there is provided a combination suitable for use in the treatment of inflammation comprising a compound of the invention as defined hereinbefore, or a pharmaceutically acceptable salt or solvate thereof, and one or more additional therapeutic agents.

Herein, where the term “combination” is used, it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt or solvate thereof, one or more additional therapeutic agents, and a pharmaceutically acceptable diluent or carrier.

### EXAMPLES

The invention will now be illustrated in the following Examples.

The following numbering system is used for the S-benzylated and N-benzylated/benzoylated derivatives described herein.

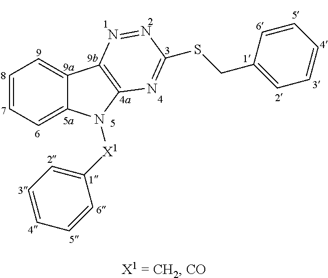

### General Experimental

All reactions involving moisture-sensitive reagents were carried out under an argon atmosphere using standard vacuum line techniques and glassware that was flame dried and cooled under argon before use. Solvents were dried according to the procedure outlined by Grubbs and co-workers (A. B. Pangborn, M. A. Giardello, R. H. Grubbs, R. K. Rosen and F. J. Timmers, Organometallics, 1996, 15, 1518). Water was purified by an Elix® UV-10 system. All other solvents and reagents were used as supplied (analytical or HPLC grade) without prior purification. Organic layers were dried over anhydrous MgSO₄. Thin layer chromatography was performed on aluminium plates coated with 60 F₂₅₄ silica. Plates were visualised using UV light (254 nm) or 1% aq KMnO₄. Melting points were recorded either on a Gallenkamp Hot Stage apparatus or a EZ-Melt Automated Melting Point Apparatus (denoted as §) and are uncorrected.

NMR spectra were recorded on Bruker Avance spectrometers in the deuterated solvent stated. The field was locked by external referencing to the relevant deuteron resonance. Chemical shifts (δ) are reported in ppm and coupling constants (J) are reported in Hz and are unaveraged.

### GENERAL PROCEDURES

### General Procedure 1

To a suspension of 5H-[1,2,4]triazino[5,6-b]indole-3-thiol (1 equiv) in methanol (5 mL) was added Et₃N (1.5 equiv). To the resulting suspension was added the requisite electrophile (1 equiv) and the reaction mixture stirred at rt for 16 h. The resulting precipitate was filtered and washed with a solution of aqueous Et₃N, which was dried in vacuo to yield the S-substituted product which was purified as specified below.

### General Procedure 2

A flask was flame dried and cooled under an atmosphere of argon. To the flask was added the S-substituted-5H-[1,2,4]triazino[5,6-b]indole-3-thiol (1 equiv) and anhydrous DMF or THF (5 mL) under argon. The resulting suspension was cooled to 0° C. using an ice bath. NaH (60% in mineral oil, 1.1-1.5 equiv) was added, resulting in a clear yellow solution, which was stirred for 15 mins at 0° C. The corresponding electrophile (1.5 equiv) was added gradually (dropwise or portionwise) at 0° C. After addition was complete, the reaction mixture was allowed to warm to rt and stirred for 16 h under argon. The reaction was quenched with water and the product extracted as specified below.

### General Procedure 3

### Suzuki Coupling of 4-bromoindoline-2,3-dione Derivatives with Trifluoroborate Potassium Salts

4-Bromoisatin (1 eq) was added to a solution of the potassium trifluoroborate salt (1.4 eq), K₃PO₄ (3.6 eq) and degassed solvent. The reaction mixture was degassed and Pd(PPh₃)₂Cl₂ or Pd(dppf)₂Cl₂ (0.1 eq) was added. The reaction vessel was sealed and heated by microwave irradiation (Biotage Initiator) for 4 h at 130° C. The reaction mixture was cooled to rt, diluted with EtOAc (˜10 mL/mmol substrate) and filtered through Celite®. The organic solution was washed with brine (˜10 mL/mmol substrate) and the resulting aqueous layer was further extracted with EtOAc (2×). The combined organic layers were dried over MgSO₄, filtered and concentrated in vacuo to give the crude product which was purified as specified below.

### General Building Block Synthesis

### 5H-[1,2,4]Triazino[5,6-b]indole-3-thiol

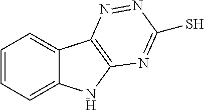 [SMILES: Sc1nnc2c(n1)[nH]c1ccccc12]

To a stirred suspension of isatin (5.00 g, 33.2 mmol) and K₂CO₃ (7.05 g, 51.0 mmol) was added thiosemicarbizide (3.09 g, 33.2 mmol). The mixture was heated to reflux for 16 h. Upon cooling the solution was acidified with glacial acetic acid to afford a precipitate, which was filtered and washed with a water/acetic acid mixture. The resulting solid was triturated with DMF, filtered and dried to yield the product as a yellow solid (6.25 g, 92%).

δ_{H} (400 MHz, DMSO-d₆, 363 K) 7.34 (1H, app t, J=7.6 Hz), 7.44 (1H, d, J=8.1 Hz), 7.62 (1H, app t, J=7.6 Hz), 8.00 (1H, d, J=7.6 Hz)

δ_{C} (100 Hz, DMSO-d₆) 113.9, 118.6, 122.7, 123.9, 132.7, 136.5, 144.0, 150.0, 179.9

mp>300° C.

### 9-Bromo-5H-[1,2,4]triazino[5,6-b]indole-3-thiol

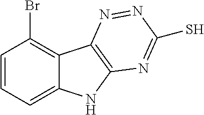 [SMILES: Sc1nnc2c(n1)[nH]c1cccc(Br)c12]

5-Bromoisatin (1.0 g, 4.45 mmol), thiosemicarbizide (405 mg, 4.45 mg) and K₂CO₃ (920 mg, 6.67 mmol) were suspended in water (50 mL). The reaction mixture was stirred at reflux for 16 h over which time the dark brown suspension became a clear light brown solution. The solution was carefully acidified by dropwise addition of AcOH and resulting precipitate was filtered. The precipitate was recrystallised from DMF to yield a red solid (1.18 g, 95%).

δ_{H} (500 MHz, DMSO-d₆, 363K) 7.43-7.52 (3H, m), 14.68 (1H, br s)

δ_{C} (125 MHz, DMSO-d₆) 113.0, 117.0, 118.3, 127.4, 133.5, 136.1, 145.3, 149.9, 180.0

mp>300° C.

### Example 1

### Preparation of 3-((2′-nitrobenzyl)thio)-5H-[1,2,4]triazino[5,6-b]indole

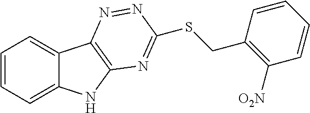 [SMILES: O=[N+]([O-])c1ccccc1CSc1nnc2c(n1)[nH]c1ccccc12]

Following General Procedure 1, 5H-[1,2,4]triazino[5,6-b]indole-3-thiol (150 mg, 0.74 mmol), 2-nitrobenzyl bromide (160 mg, 0.74 mmol) and Et₃N (155 μL, 1.11 mmol) in methanol (5 mL) were reacted. The resulting precipitate was filtered and washed with Et₃N/H₂O to give a pale yellow solid (246 mg, 98%).

δ_{H} (400 MHz, DMSO-d₆, 363 K) 4.87 (2H, s), 7.40-7.43 (1H, m), 7.53-7.59 (2H, m), 7.66-7.72 (2H, m), 7.91 (1H, d, J=7.7 Hz), 8.05 (1H, d, J=7.7 Hz), 8.28 (1H, m), 12.62 (1H, br s).

δ_{C} (100 Hz, DMSO-d₆) δ1.1, 112.4, 117.5, 121.5, 122.5, 124.9, 127.2, 128.9, 131.0, 132.5, 132.9, 133.5, 140.4, 141.3, 146.5, 166.0

mp 264-265° C.

### Example 2

### Preparation of 3-((2′-Cyanobenzyl)thio)-5H-[1,2,4]triazino[5,6-b]indole

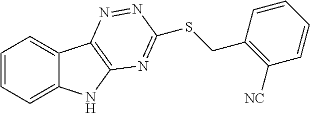 [SMILES: N#Cc1ccccc1CSc1nnc2c(n1)[nH]c1ccccc12]

Following General Procedure 1, to 5H-[1,2,4]triazino[5,6-b]indole-3-thiol (2.26 g, 11.2 mmol) and Et₃N (2.33 mL, 16.8 mmol) in methanol (40 mL) was added 2-cyano benzyl bromide (2.19 g, 11.2 mmol) and the resulting mixture stirred at rt for 16 h. The resulting precipitate was filtered and washed with Et₃N/H₂O to give a pale yellow solid (3.60 g, quant).

δ_{H} (400 MHz, DMSO-d₆, 363 K) 4.73 (2H, s), 7.41-7.49 (2H, m), 7.58 (1H, d, J=7.8 Hz), 7.64-7.71 (2H, m), 7.82 (1H, m), 7.85 (1H, d, J=7.6 Hz), 8.30 (1H, d, J=7.8 Hz), 12.66 (1H, br s)

δ_{C} (100 MHz, DMSO-d₆) δ3.4, 112.9, 113.6, 118.3, 118.4, 122.4, 123.4, 129.1, 131.1, 131.9, 134.0, 134.2, 141.2, 142.1, 142.2, 147.4, 166.6

mp 276-278° C.

### Example 3

### Preparation of 2′-(((5-Benzyl-5H-[1,2,4]triazino[5,6-b]indol-3-yl)thio)methyl)benzonitrile

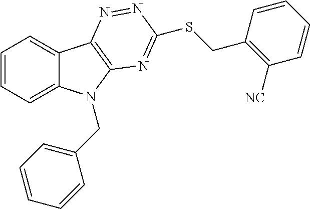 [SMILES: N#Cc1ccccc1CSc1nnc2c3ccccc3n(Cc3ccccc3)c2n1]

Following General Procedure 2, to 3-((2′-cyanobenzyl)thio)-5H-[1,2,4]triazino[5,6-b]indole (50 mg, 0.15 mmol) in THF at 0° C., was added NaH (10 mg, 0.24 mmol) followed by benzyl bromide (25 μL, 0.24 mmol) and the resulting mixture stirred for 16 h. The reaction mixture was quenched with water (30 mL) and CH₂Cl₂ (30 mL) added. The organic layer was separated and washed with brine (30 mL), dried over MgSO₄, filtered and concentrated in vacuo to yield a light yellow solid (49 mg, 80%).

δ_{H} (500 MHz, DMSO-d₆, 363 K) 4.77 (2H, s), 5.68 (2H, s), 7.23-7.33 (5H, m), 7.43 (1H, app td, J=7.7, 1.1 Hz), 7.47-7.51 (1H, m), 7.54 (1H, app td, J=7.7, 1.4 Hz), 7.69-7.74 (2H, m), 7.75-7.81 (2H, m), 8.37 (1H, d, J=7.9 Hz)

δ_{C} (125 MHz, DMSO-d₆) δ2.4, 44.2, 111.7, 111.9, 117.5, 117.6, 119.3, 121.7, 123.2, 127.3, 127.7, 128.1, 128.8, 130.2, 133.1, 133.2, 135.8, 140.7, 141.2, 141.5, 146.2, 166.2

mp 197-200° C.

### Example 4

### Preparation of 5-Benzyl-3-(2′-fluorobenzylthio)-5H-[1,2,4]triazino[5,6-b]indole

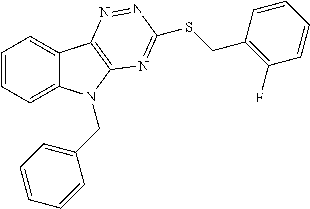 [SMILES: Fc1ccccc1CSc1nnc2c3ccccc3n(Cc3ccccc3)c2n1]

Following General Procedure 2, to 3-((2′-fluorobenzyl)thio)-5H-[1,2,4]triazino[5,6-b]indole (50 mg, 0.16 mmol) in THF was added NaH (10 mg, 0.24 mmol) followed by benzyl bromide (25 μL, 0.24 mmol) at 0° C. and the resulting mixture stirred for 16 h. The reaction mixture was quenched with water (30 mL) and CH₂Cl₂ (30 mL) added. The organic layer was separated and washed with brine (30 mL), dried over MgSO₄, filtered and concentrated in vacuo to yield a light yellow solid (26 mg, 40%).

δ_{H} (500 MHz, DMSO-d₆, 363 K) 4.62 (2H, s), 5.67 (2H, s), 7.04 (1H, app td, J=7.5, 1.1 Hz), 7.18 (1H, app t, J=7.6 Hz), 7.25-7.35 (6H, m), 7.49 (1H, app t, J=6.9 Hz), 7.57 (1H, app td, J=7.7, 1.6 Hz), 7.68-7.76 (2H, m), 8.37 (1H, d, J=7.9 Hz)

δ_{C} (125 MHz, DMSO-d₆) 27.5, 44.2, 111.6, 115.3 (d, J=21 Hz), 117.6, 121.7, 123.1, 124.4 (d, J=2.9 Hz), 124.7, 127.3, 127.8, 128.8, 129.4 (d, J=8.6 Hz), 131.0, 131.3 (d, J=3.8 Hz), 135.9, 140.7 (d, J=31 Hz), 141.0, 146.2, 160.5 (d, J=246 Hz), 166.7

δ_{F} (470 MHz, DMSO-d₆)-116.9

mp 139-141° C.

### Example 5

### Preparation of 2′(((5-(4″-Chlorobenzyl)-5H-[1,2,4]triazino[5,6-b]indol-3-yl)thio)methyl)benzonitrile

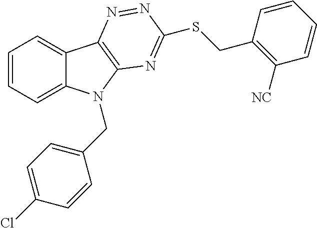 [SMILES: N#Cc1ccccc1CSc1nnc2c3ccccc3n(Cc3ccc(Cl)cc3)c2n1]

Following General Procedure 2, to 3-((2′-cyanobenzyl)thio)-5H-[1,2,4]triazino[5,6-b]indole (100 mg, 0.32 mmol) in THF (5 mL) was added NaH (19 mg, 0.47 mmol) followed by 4-chloro benzyl bromide (97 mg, 0.47 mmol) at 0° C. and the resulting mixture stirred for 16 h. The reaction mixture was quenched with water (30 mL) and CH₂Cl₂ (30 mL) added. The organic layer was separated and washed with brine (30 mL), dried over MgSO₄, filtered and concentrated in vacuo to yield a yellow solid (82 mg, 58%).

δ_{H} (500 MHz, DMSO-d₆, 363 K) 4.75 (2H, s), 5.66 (2H, s), 7.28 (2H, d, J=8.6 Hz), 7.34 (2H, d, J=8.4 Hz), 7.42 (1H, app t, J=7.6 Hz), 7.47 (1H, app t, J=7.6 Hz), 7.55 (1H, app t, J=7.6 Hz), 7.65-7.80 (4H, m), 8.35 (1H, d, J=7.8 Hz)

δ_{C} (125 MHz, DMSO-d₆) δ3.2, 44.4, 112.4, 112.8, 118.5, 122.6, 124.1, 129.0, 129.6, 130.1, 131.1, 131.9, 133.2, 133.9, 134.1, 135.7, 141.5, 142.0, 142.3, 142.4, 147.0, 167.1

mp 178-179° C.

### Example 6

### Preparation of 5-(4″-Chlorobenzyl)-3-((2′-nitrobenzyl)thio)-5H-[1,2,4]triazino[5,6-b]indole

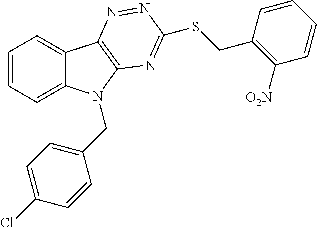 [SMILES: O=[N+]([O-])c1ccccc1CSc1nnc2c3ccccc3n(Cc3ccc(Cl)cc3)c2n1]

Following General Procedure 2, to 3-((2′-nitrobenzyl)thio)-5H-[1,2,4]triazino[5,6-b]indole (100 mg, 0.30 mmol) in THF (5 mL) was added NaH (18 mg, 0.45 mmol) followed by 4-chloro benzyl bromide (97 mg, 0.45 mmol) at 0° C. and the resulting mixture stirred for 16 h. The reaction mixture was quenched with water (30 mL) and CH₂Cl₂ (30 mL) added. The organic layer was separated and washed with brine (30 mL), dried over MgSO₄, filtered and concentrated in vacuo to yield a yellow solid (76 mg, 55%).

δ_{H} (500 MHz, DMSO-d₆, 363K) 5.08 (2H, s), 5.57 (2H, s), 7.17-7.39 (6H, m), 7.49-7.55 (3H, m), 7.92 (1H, dd, J=7.6, 1.1 Hz), 7.99-8.03 (1H, dd, J=7.9, 1.0 Hz), 8.43 (1H, dd, J=7.8, 0.8 Hz)

δ_{C} (125 MHz, pyr-d₅) 32.5, 45.1, 111.6, 122.3, 123.5, 125.6, 127.8, 128.5, 129.0, 129.5, 131.3, 133.2, 133.8, 134.5, 136.4, 141.4, 142.1, 142.4, 147.0, 153.9, 166.9

mp 100-102° C.

### Example 7

### Preparation of (3-((2′-Nitrobenzyl)thio)-5H-[1,2,4]triazino[5,6-b]indol-5-yl)(phenyl)methanone

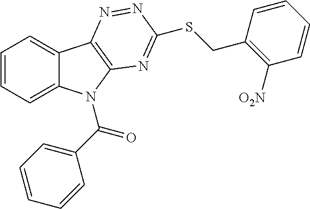 [SMILES: O=C(c1ccccc1)n1c2ccccc2c2nnc(SCc3ccccc3[N+](=O)[O-])nc21]

To a stirred suspension of 3-((2′-nitrobenzyl)thio)-5H-[1,2,4]triazino[5,6-b]indole (100 mg, 0.30 mmol), DMAP (0.7 mg, 0.05 mmol) and Et₃N (82 μL, 0.59 mmol) in CH₂Cl₂ (5 mL) at rt was added benzoyl bromide (51 μL, 0.45 mmol). The resulting mixture was stirred at rt for 16 h. CH₂Cl₂ (30 mL) was added to the suspension and washed with H₂O (30 mL). The organic layer was washed with brine (30 mL), dried over MgSO₄, filtered and concentrated in vacuo. The resulting solid was washed with methanol to yield a white solid (96 mg, 73%).

δ_{H} (500 MHz, pyr-d₅, 363K) 4.53 (2H, s), 7.37 (1H, app t, J=7.7 Hz), 7.42 (1H, d, J=7.6 Hz), 7.47-7.63 (5H, m), 7.71 (1H, app t, J=8.0), 8.04-8.11 (3H, m), 8.41-8.49 (2H, m)

δ_{C} (125 MHz, pyr-d₅) 33.6, 111.7, 116.3, 119.7, 122.2, 125.6, 127.5, 128.3, 129.9, 130.1, 130.9, 133.4, 134.6, 135.2, 135.5, 136.3, 141.7, 143.6 149.2, 167.1, 170.0

mp 209-210° C.

### Example 8

### Preparation of 2′-(((9-Bromo-5H-[1,2,4]triazino[5,6-b]indol-3-yl)thio)methyl)benzonitrile

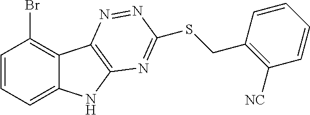 [SMILES: N#Cc1ccccc1CSc1nnc2c(n1)[nH]c1cccc(Br)c12]

Following General Procedure 1,9-bromo-5H-[1,2,4]triazino[5,6-b]indol-3-thiol (100 mg, 0.36 mmol), 2-cyanobenzyl bromide (70 mg, 0.36 mmol) and Et₃N (75 μL, 0.54 mmol) in methanol (5 mL) were reacted together. The resulting precipitate was filtered and washed with Et₃N/H₂O to give a light yellow solid (91 mg, 64%).

δ_{H} (400 MHz, DMSO-d₆, 363K) 4.76 (2H, s), 7.48 (1H, app td, J=7.6, 1.0 Hz), 7.55-7.64 (3H, m), 7.67 (1H, app td, J=7.7, 1.3 Hz), 7.83 (1H, d, J=7.8 Hz), 7.88 (1H, dd, J=7.8, 1.0 Hz), 12.89 (1H, br s)

δ_{C} (100 MHz, DMSO-d₆) δ3.4, 112.8, 112.9, 116.9, 117.9, 118.3, 127.0, 129.1, 131.2, 132.5, 134.0, 134.2, 141.9, 142.5, 147.3, 149.1, 167.0

mp 283-284° C.

### Example 9

### Preparation of 9-Bromo-3-((2′-nitrobenzyl)thio)-5H-[1,2,4]triazino[5,6-b]indole

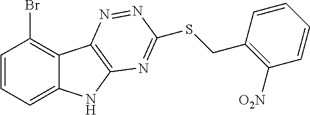 [SMILES: O=[N+]([O-])c1ccccc1CSc1nnc2c(n1)[nH]c1cccc(Br)c12]

Following General Procedure 1,9-bromo-5H-[1,2,4]triazino[5,6-b]indol-3-thiol (100 mg, 0.36 mmol), 2-cyanobenzyl bromide (70 mg, 0.36 mmol) and Et₃N (75 μL, 0.54 mmol) in methanol (5 mL) were reacted. The resulting precipitate was filtered and washed with Et₃N/H₂O to give a light yellow solid (98 mg, 66%).

δ_{H} (400 MHz, DMSO-d₆, 363K) 4.90 (2H, s), 7.42-7.62 (4H, m), 7.69 (1H, app t, J=7.6, 1.3 Hz), 7.93 (1H, dd, J=7.8, 1.3 Hz), 8.06 (1H, dd, J=8.1, 1.3 Hz)

δ_{C} (100 MHz, DMSO-d₆) δ2.0, 112.6, 116.9, 118.1, 125.8, 126.7, 128.5, 129.7, 132.3, 133.3, 134.6, 141.2, 142.4, 147.2, 149.2, 167.1

mp 285-286° C.

### Example 10

### Preparation of 2′-(((5-(2″-Nitrobenzyl)-5H-[1,2,4]triazino[5,6-b]indol-3-yl)thio)methyl)benzonitrile

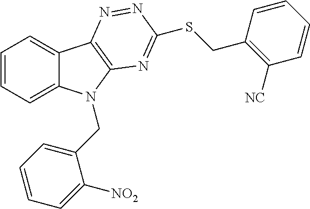 [SMILES: N#Cc1ccccc1CSc1nnc2c3ccccc3n(Cc3ccccc3[N+](=O)[O-])c2n1]

Following General Procedure 2, to 2′(((5H-[1,2,4]triazino[5,6-b]indol-3-yl)thio)methyl)benzonitrile (60 mg, 0.19 mmol) in THF (5 mL) was added NaH (11 mg, 0.28 mmol) followed by 2-nitro benzyl bromide (40 mg, 0.0.21 mmol) at 0° C. and the resulting mixture stirred for 16 h. The reaction mixture was quenched with water until a precipitate formed. The precipitate was filtered and dried to give an off white solid (79 mg, 96%).

δ_{H} (500 MHz, pyr-d₆, 363K) 4.65 (2H, s), 6.02 (2H, s), 6.71 (1H, d, J=7.1 Hz), 7.42-7.72 (7H, m), 7.81-7.89 (2H, m), 8.31 (1H, d, J=7.8 Hz), 8.40 (1H, d, J=7.6 Hz)

δ_{C} (125 MHz, pyr-d₆) 35.0, 45.2, 112.9, 115.0, 119.3, 119.9, 120.7, 124.0, 126.7, 129.3, 129.8, 130.4, 132.2, 133.0, 134.8, 134.8, 135.4, 141.1, 142.7, 143.7, 143.9, 148.9, 155.1, 169.2

mp 225-226° C. (dec)

### Example 11

### Preparation of 3-((2′,4′-Dinitrobenzyl)thio)-5H-[1,2,4]triazino[5,6-b]indole

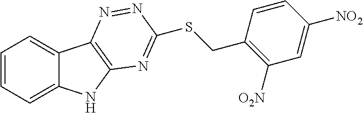 [SMILES: O=[N+]([O-])c1ccc(CSc2nnc3c(n2)[nH]c2ccccc23)c([N+](=O)[O-])c1]

Following General Procedure 1, 5H-[1,2,4]triazino[5,6-b]indole-3-thiol (100 mg, 0.50 mmol), 2,4-dinitrobenzyl bromide (129 mg, 0.50 mmol) and Et₃N (103 μL, 0.74 mmol) in methanol (5 mL) were reacted. The resulting precipitate was filtered and washed with Et₃N/H₂O to give a faint yellow solid (132 mg, 70%).

δ_{C} (500 MHz, DMSO-d₆, 363K) 4.95 (2H, s), 7.43 (1H, app t, J=7.3 Hz), 7.59 (1H, d, J=8.0 Hz), 7.70 (1H, app t, J=7.6 Hz), 8.19 (1H, d, J=8.6 Hz), 8.29 (1H, d, J=8.0 Hz), 8.49 (1H, dd, J=8.6, 2.5 Hz), 8.74 (1H, d, J=2.5 Hz), 12.63 (1H, br s)

δ_{C} (125 MHz, DMSO-d₆) δ0.8, 112.8, 117.5, 120.2, 121.6, 122.6, 127.5, 131.1, 134.0, 140.3, 140.5, 141.5, 146.5, 146.6, 148.4, 165.3

mp 255-259° C.

### Example 12

### Preparation of 3-((2′-Fluoro-6′-nitrobenzyl)thio)-5H-[1,2,4]triazino[5,6-b]indole

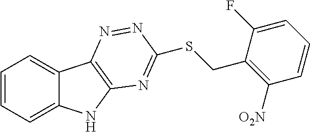 [SMILES: O=[N+]([O-])c1cccc(F)c1CSc1nnc2c(n1)[nH]c1ccccc12]

Following General Procedure 1, 5H-[1,2,4]triazino[5,6-b]indole-3-thiol (100 mg, 0.50 mmol), 2-fluoro-6-nitrobenzyl bromide (116 mg, 0.50 mmol) and Et₃N (103 μL, 0.74 mmol) in methanol (5 mL) were reacted. The resulting precipitate was filtered and washed with Et₃N/H₂O to give a faint yellow solid (135 mg, 75%).

δ_{H} (500 MHz, DMSO-d₆, 363K) 4.86 (2H, s), 7.40 (1H, app t, J=8.0 Hz), 7.53-7.72 (4H, m), 7.87 (1H, d, J=8.1 Hz), 8.28 (1H, d, J=7.8 Hz), 12.57 (1H, br s)

δ_{C} (125 MHz, DMSO-d₆) 24.8 (d, J=3.2 Hz), 113.6, 118.4, 121.7 (d, J=22 Hz), 121.8 (d, J=3.2 Hz), 121.9 (d, J=17 Hz), 122.4, 123.4, 130.9 (d, J=9.6 Hz), 131.8, 141.3, 142.3, 147.3, 150.5 (d, J=7.4 Hz), 160.2, 164.6 (d, J=250 Hz)

δ_{F} (470 MHz, DMSO-d₆) −110.5

mp 271-172° C.

### Example 13

### Preparation of 5-(2″-Fluorobenzyl)-3-(2′-nitrobenzyl)thio)-5H-[1,2,4]triazino[5,6-b]indole

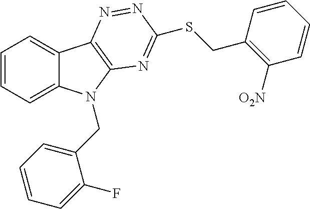 [SMILES: O=[N+]([O-])c1ccccc1CSc1nnc2c3ccccc3n(Cc3ccccc3F)c2n1]

Following General Procedure 2, to 3-((2′-nitrobenzyl)thio)-5H-[1,2,4]triazino[5,6-b]indole (60 mg, 0.18 mmol) in THF (5 mL) was added NaH (10.7 mg, 0.27 mmol) followed by 2-fluoro benzyl bromide (22 μL, 0.18 mmol) at 0° C. and stirred for 16 h. The reaction mixture was quenched with water until a precipitate formed. The precipitate was filtered and dried to give a faint yellow solid (50 mg, 63%).

δ_{H} (500 MHz, DMSO-d₆, 363K) 4.87 (2H, s), 5.70 (2H, s), 7.09 (1H, app td, J=7.6, 1.0 Hz), 7.15 (1H, app td, J=7.6, 1.6 Hz), 7.26 (1H, app t, J=8.3 Hz), 7.35-7.39 (1H, m), 7.47-7.53 (3H, m), 7.70 (1H, d, J=7.9 Hz), 7.73 (1H, app td, J=8.2, 1.3 Hz), 7.78-7.80 (1H, m), 8.02-8.04 (1H, m), 8.37 (1H, d, J=7.6 Hz), 12.62 (1H, br s)

δ_{C} (125 MHz, DMSO-d₆) δ1.1, 45.3, 111.4, 115.6 (d, J=21 Hz′), 117.6, 121.7, 122.4 (d, J=14 Hz), 123.2, 124.8 (d, J=2.9 Hz), 129.4 (d, J=2.9 Hz), 129.5, 130.0 (d, J=8.6 Hz), 130.1, 131.1, 132.3, 133.3, 133.6, 140.7, 141.1, 146.2, 148.3, 160.1 (q, J=245 Hz), 166.4

δ_{F} (470 MHz, DMSO-d₆)-117.2

mp 106-108° C.

### Example 14

### Preparation of 5-Benzyl-3-((2′-nitrobenzyl)thio)-5H-[1,2,4]triazino[5,6-b]indole

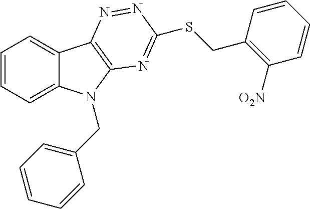 [SMILES: O=[N+]([O-])c1ccccc1CSc1nnc2c3ccccc3n(Cc3ccccc3)c2n1]

Following General Procedure 2, to 3-((2′-nitrobenzyl)thio)-5H-[1,2,4]triazino[5,6-b]indole (60 mg, 0.18 mmol) in THF (5 mL) was added NaH (11 mg, 0.27 mmol) followed by benzyl bromide (21 μL, 0.18 mmol) at 0° C. and the resulting mixture stirred for 16 h. The reaction mixture was quenched with water until a precipitate formed. The precipitate was filtered and dried to give a faint yellow solid (63 mg, 82%).

δ_{H} (500 MHz, DMSO-d₆, 363K) 4.89 (2H, s), 5.66 (2H, s), 7.25-7.34 (5H, m), 7.47 (1H, tt, J=7.9, 2.2 Hz), 7.49-7.53 (2H, m), 7.67-7.74 (2H, m), 7.81 (1H, dd, J=5.7, 3.5 Hz), 8.03 (1H, dd, J=6.0, 3.8 Hz), 8.35 (1H, d, J=7.6 Hz)

δ_{C} (125 MHz, DMSO-d₆) δ1.2, 44.2, 111.6 117.5 121.7, 123.1, 125.0, 127.2, 127.7, 128.8, 128.9, 131.0, 132.4, 133.3, 133.6, 135.8, 140.8, 141.1, 146.1, 148.3, 166.4

mp 160-162° C.

### Example 15

### Preparation of 3-((2′-Methylbenzyl)thio)-5H-[1,2,4]triazino[5,6-b]indole

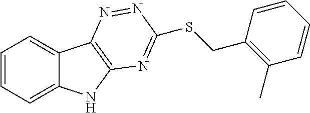 [SMILES: Cc1ccccc1CSc1nnc2c(n1)[nH]c1ccccc12]

Following General Procedure 1, 5H-[1,2,4]triazino[5,6-b]indole-3-thiol (200 mg, 0.99 mmol), 2-methylbenzyl bromide (132 μL, 0.99 mmol) and Et₃N (206 μL, 1.49 mmol) in methanol (5 mL) were reacted. The resulting precipitate was filtered and washed with Et₃N/H₂O to give a faint yellow solid (235 mg, 77%).

δ_{H} (400 MHz, DMSO-d₆, 363K) 2.42 (3H, s), 4.56 (2H, s), 7.11-7.24 (3H, m), 7.42 (1H, app t, J=7.6 Hz), 7.49 (1H, d, J=7.8 Hz), 7.57 (1H, d, J=8.1 Hz), 7.68 (1H, app td, J=7.7 Hz, 1.0 Hz), 8.30 (1H, d, J=7.8 Hz), 12.65 (1H, br s)

δ_{C} (100 MHz, DMSO-d₆) 19.8, 33.3, 113.6, 118.5, 122.3, 123.3, 126.9, 128.4, 130.8, 131.2, 131.7, 135.6, 137.6, 141.2, 142.0, 147.5, 167.7

mp 202-203° C.

### Example 16

### Preparation of 2′-(((5-(4″-Dimethylamino)benzoyl)-5H-[1,2,4]triazino[5,6-b]indol-3-yl)thio)methyl)benzonitrile

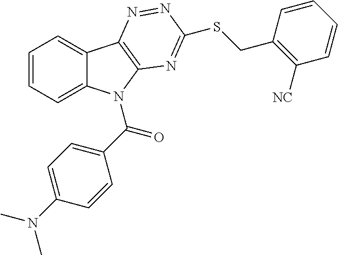 [SMILES: CN(C)c1ccc(C(=O)n2c3ccccc3c3nnc(SCc4ccccc4C#N)nc32)cc1]

To a stirred suspension of 2′(((5H-[1,2,4]triazino[5,6-b]indol-3-yl)thio)methyl)benzonitrile (100 mg, 0.32 mmol), DMAP (0.8 mg, 0.06 mmol) and Et₃N (86 μL, 0.63 mmol) in CH₂Cl₂ (5 mL) at rt was added 4-dimethylaminobenzoyl chloride (83 mg, 0.48 mmol). The resulting mixture was stirred at rt for 16 h. CH₂Cl₂ (30 mL) was added to the suspension and washed with H₂O (30 mL). The organic layer was washed with brine (30 mL), dried over MgSO₄, filtered and concentrated in vacuo. The resulting solid was washed with methanol to yield a white solid (81 mg, 55%).

δ_{H} (400 MHz, pyr-d₅, 363K) 2.84 (6H, s), 5.04 (2H, s), 6.74 (2H, d, J=9.1 Hz), 7.26-7.30 (1H, m), 7.44-7.47 (1H, m), 7.56 (1H, app t, J=7.6 Hz), 7.59 (1H, d, J=7.6 Hz), 7.62-7.71 (2H, m), 8.08 (2H, d, J=9.1 Hz), 8.19 (1H, d, J=7.8 Hz), 8.48 (1H, d, J=7.8 Hz)

δ_{C} (125 MHz, pyr-d₅) 33.5, 39.7, 111.2, 113.2, 116.2, 118.1, 119.4, 120.2, 121.9, 125.1, 128.4, 131.1, 131.5, 133.2, 133.5, 134.0, 140.9, 141.7, 142.4, 147.6, 154.9, 166.8, 168.0

mp 196-197° C.

### Example 17

### Preparation of 2′-((5-(3″,4″-Dimethoxybenzoyl)-5H-[1,2,4]triazino[5,6-b]indol-3-yl)thio)methyl)benzonitrile

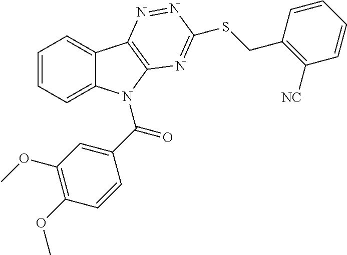 [SMILES: COc1ccc(C(=O)n2c3ccccc3c3nnc(SCc4ccccc4C#N)nc32)cc1OC]

To a stirred suspension of 2′(((5H-[1,2,4]triazino[5,6-b]indol-3-yl)thio)methyl)benzonitrile (100 mg, 0.32 mmol), DMAP (0.8 mg, 0.06 mmol) and Et₃N (86 μL, 0.63 mmol) in CH₂Cl₂ (5 mL) at rt was added 3,4-dimethoxybenzoyl chloride (95 mg, 0.48 mmol). The resulting mixture was stirred at rt for 16 h. CH₂Cl₂ (30 mL) was added to the suspension and washed with water (30 mL). The organic layer was washed with brine (30 mL), dried over MgSO₄, filtered and concentrated in vacuo. The resulting solid was washed with methanol to yield a white solid (87 mg, 56%).

δ_{H} (500 MHz, DMSO-d₆, 363 K) 3.73 (3H, s), 3.80 (3H, s), 4.25 (2H, s), 7.09 (1H, d, J=8.5 Hz), 7.35 (1H, d, J=7.9 Hz), 7.44-7.48 (1H, m), 7.55 (1H, d, J=1.9 Hz), 7.58-7.61 (1H, m), 7.63-7.67 (2H, m), 7.80-7.85 (2H, m), 8.14 (1H, d, J=8.5 Hz), 8.43 (1H, d, J=7.9 Hz)

δ_{C} (125 MHz, DMSO-d₆) δ2.1, 55.8, 55.9, 110.8, 111.7, 113.4, 115.9, 117.2, 119.5, 121.4, 125.1, 125.3, 125.5, 128.3, 130.2, 131.5, 133.2, 133.2, 139.7, 140.5, 142.2, 147.3, 148.4, 153.8, 166.3, 166.8

mp 234-235° C.

### Example 18

### Preparation of 2′-(((5-(Cyclohexanecarbonyl)-5H-[1,2,4]triazino[5,6-b]indol-3-yl)thio)methyl)benzonitrile

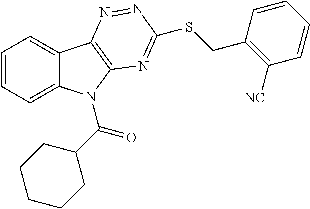 [SMILES: N#Cc1ccccc1CSc1nnc2c3ccccc3n(C(=O)C3CCCCC3)c2n1]

Following General Procedure 2, to 2′(((5H-[1,2,4]triazino[5,6-b]indol-3-yl)thio)methyl)benzonitrile (100 mg, 0.32 mmol) in DMF (5 mL) was added NaH (13.8 mg, 0.35 mmol) followed by cyclohexane carbonyl chloride (63 μL, 0.47 mmol) at 0° C. and the resulting mixture stirred for 16 h. The reaction mixture was quenched with water until a precipitate formed. The precipitate was filtered and dried to give a faint yellow solid (98 mg, 71%).

δ_{H} (500 MHz, DMSO-d₆, 363K) 1.18-1.38 (4H, m), 1.45-1.55 (2H, m), 1.75-1.83 (2H, m) 2.02-2.07 (2H, m), 4.04-4.12 (1H, m), 4.84 (2H, s), 7.53 (1H, app td, J=7.6, 1.1 Hz), 7.60-7.65 (1H, m), 7.71 (1H, app td, J=7.6, 1.1 Hz) 7.77-7.85 (2H, m), 7.89-7.93 (1H, m), 8.38 (1H, d, J=6.9 Hz), 8.59 (1H, d, J=8.5 Hz)

δ_{C} (125 MHz, DMSO-d₆) 25.1, 25.3, 28.5, 32.8, 44.7, 112.0, 112.8, 117.4, 117.5, 119.6, 121.1, 125.7, 128.5, 130.4, 132.1, 133.6, 139.6, 140.3, 142.6, 147.0, 166.5, 176.2

mp 194-197° C.

### Example 19

### Preparation of 2′-(((5-(3″-Trifluoromethyl)benzoyl)-5H-[1,2,4]triazino[5,6-b]indol-3-yl)thio)methyl)benzonitrile

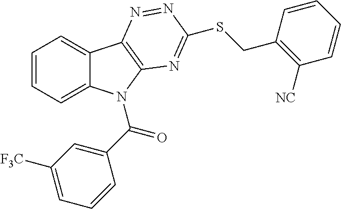 [SMILES: N#Cc1ccccc1CSc1nnc2c3ccccc3n(C(=O)c3cccc(C(F)(F)F)c3)c2n1]

Following General Procedure 2, to 2′(((5H-[1,2,4]triazino[5,6-b]indol-3-yl)thio)methyl)benzonitrile (100 mg, 0.32 mmol) in THF (5 mL) was added NaH (13.8 mg, 0.35 mmol) followed by 3-(trifluoromethyl)benzoyl chloride (71 μL, 0.47 mmol) at 0° C. and the resulting mixture stirred for 16 h. The reaction mixture was quenched with water until a precipitate formed. The precipitate was filtered and dried to give a faint yellow solid (89 mg, 58%).

δ_{H} (500 MHz, DMSO-d₆, 363K) 4.74 (2H, s), 7.36 (1H, d, J=8.0 Hz), 7.46-7.68 (4H, m) 7.73-7.78 (2H, m), 7.95 (1H, s), 8.18-8.23 (2H, m), 8.40 (1H, d, J=8.4 Hz), 8.43 (1H, d, J=7.3 Hz)

δ_{C} (125 MHz, DMSO-d₆) δ1.8, 111.7, 116.6, 117.1, 119.8, 121.4, 123.7 (q, J=273 Hz), 125.9, 126.6 (q, J=3.8 Hz), 128.4, 129.1 (q, J=3.8 Hz), 129.1 (q, J=32 Hz), 129.5, 130.2, 131.8, 133.2, 133.4, 133.6, 135.3, 139.4, 140.0, 142.5, 147.4, 166.2, 166.6

δ_{F} (470 MHz, DMSO-d₆) −61.2

mp 155-156° C.

### Example 20

### Preparation of 2′-(((5-(4″-Fluorobenzoyl)-5H-[1,2,4]triazino[5,6-b]indol-3-yl)thio)methyl)benzonitrile

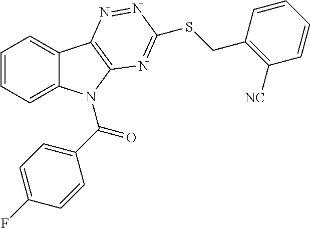 [SMILES: N#Cc1ccccc1CSc1nnc2c3ccccc3n(C(=O)c3ccc(F)cc3)c2n1]

Following General Procedure 2, to 2′(((5H-[1,2,4]triazino[5,6-b]indol-3-yl)thio)methyl)benzonitrile (100 mg, 0.32 mmol) in DMF (5 mL) was added NaH (13.8 mg, 0.35 mmol) followed by 4-fluoro benzoyl bromide (56 μL, 0.47 mmol) at 0° C. and the resulting mixture stirred for 16 h. The reaction mixture was quenched with water until a precipitate formed. The precipitate was filtered and dried to give a faint yellow solid (95 mg, 67%).

δ_{H} (500 MHz, DMSO-d₆, 363K) 4.16 (2H, s), 7.34 (2H, app t, J=8.8 Hz), 7.41 (1H, d, J=7.9 Hz), 7.46-7.50 (1H, m), 7.60-7.64 (1H, m), 7.66 (1H, app t, J=7.6 Hz), 7.82-7.86 (2H, m), 7.98-8.06 (2H, m), 8.28 (1H, d, J=7.8 Hz), 8.43 (1H, d, J=7.3 Hz)

δ_{C} (125 MHz, DMSO-d₆) δ1.9, 111.7, 115.5 (d, J=23 Hz), 116.3, 117.2, 119.7, 121.4, 125.6, 128.4, 130.2, 130.3 (d, J=2.9 Hz), 131.7, 133.2, 133.2 (d, J=9.5 Hz), 133.4, 139.5, 140.1, 142.2, 147.3, 165.1 (d, J=253 Hz), 166.4, 166.7

δ_{F} (470 MHz, DMSO-d₆) −110.4

mp 202-204° C.

### Example 21

### Preparation of 2′-(((5-(4″-(Trifluoromethoxy)benzoyl)-5H-[1,2,4]triazino[5,6-b]indol-3-yl)thio)methyl)benzonitrile

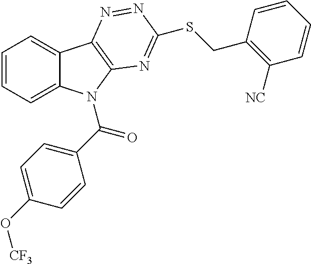 [SMILES: N#Cc1ccccc1CSc1nnc2c3ccccc3n(C(=O)c3ccc(OC(F)(F)F)cc3)c2n1]

Following General Procedure 2, to 2′(((5H-[1,2,4]triazino[5,6-b]indol-3-yl)thio)methyl)benzonitrile (100 mg, 0.32 mmol) in DMF (5 mL) was added NaH (13.8 mg, 0.35 mmol) followed by 4-(trifluoromethoxy)benzoyl chloride (74 μL, 0.47 mmol) at 0° C. and the resulting mixture stirred for 16 h. The reaction mixture was quenched with water until a precipitate formed. The precipitate was filtered and dried to give a white solid (109 mg, 67%).

δ_{H} (500 MHz, DMSO-d₆, 363K) 4.08 (2H, s), 7.39 (1H, d, J=7.6 Hz), 7.45-7.53 (3H, m), 7.61 (1H, app td, J=7.7, 1.2 Hz), 7.68 (1H, app t, J=7.4 Hz), 7.81-7.88 (2H, m), 8.08-8.14 (2H, m), 8.37 (1H, d, J=7.9 Hz), 8.43 (1H, d, J=7.3 Hz)

δ_{C} (125 MHz, DMSO-d₆) δ2.5, 111.7, 116.5, 117.1, 119.7, 120.4, 120.7, 121.3, 125.8, 128.5, 129.5 (q, J=258 Hz), 130.3, 131.7, 132.4, 133.1, 133.4, 139.4, 140.0, 142.2, 147.3, 151.4, 166.6, 170.3

δ_{F} (470 MHz, DMSO-d₆) −56.8

mp 169-170° C.

### Example 22

### Preparation of 2′-(((5-(Cyclopentanecarbonyl)-5H-[1,2,4]triazino[5,6-b]indol-3-yl)thio)methyl)benzonitrile

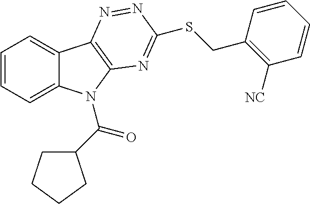 [SMILES: N#Cc1ccccc1CSc1nnc2c3ccccc3n(C(=O)C3CCCC3)c2n1]

Following General Procedure 2, to 2′(((5H-[1,2,4]triazino[5,6-b]indol-3-yl)thio)methyl)benzonitrile (100 mg, 0.32 mmol) in DMF (5 mL) was added NaH (13.8 mg, 0.35 mmol) followed by cyclopentane carbonyl chloride (58 μL, 0.47 mmol) at 0° C. and the resulting mixture stirred for 16 h. The reaction mixture was quenched with water until a precipitate formed. The precipitate was filtered and dried to give a faint yellow solid (56 mg, 42%).

δ_{H} (500 MHz, DMSO-d₆, 363K) 1.56-1.64 (2H, m), 1.66-1.74 (2H, m), 1.87-2.03 (4H, m), 4.35-4.42 (1H, m), 4.81 (2H, s), 7.52 (1H, app t, J=7.6 Hz), 7.62 (1H, app t, J=7.6 Hz), 7.69-7.73 (1H, m), 7.79 (1H, app t, J=7.9 Hz), 7.8 (1H, d), 7.92 (1H, d, J=7.6 Hz), 8.38 (1H, d, J=7.6 Hz), 8.58 (1H, d, J=8.5 Hz)

δ_{C} (125 MHz, DMSO-d₆) 25.6, 29.5, 32.7, 45.9, 111.9, 117.4, 117.5, 119.5, 121.1, 125.7, 128.4, 130.4, 132.0, 133.2, 133.6, 139.6, 140.2, 142.5, 147.0, 166.6, 176.3

mp 173-174° C.

### Example 23

### Preparation of 2′-(((5-(Furan-2″-carbonyl)-5H-[1,2,4]triazino[5,6-b]indol-3-yl)thio)methyl)benzonitrile

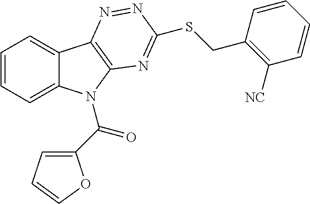 [SMILES: N#Cc1ccccc1CSc1nnc2c3ccccc3n(C(=O)c3ccco3)c2n1]

Following General Procedure 2, to 2′(((5H-[1,2,4]triazino[5,6-b]indol-3-yl)thio)methyl)benzonitrile (100 mg, 0.32 mmol) in DMF (5 mL) was added NaH (13.8 mg, 0.35 mmol) followed by 2-furfuryl chloride (46 μL, 0.47 mmol) at 0° C. and the resulting mixture stirred for 16 h. The reaction mixture was quenched with water until a precipitate formed. The precipitate was filtered and dried to give a faint yellow solid (103 mg, 78%).

δ_{H} (500 MHz, DMSO-d₆, 363K) 4.49 (2H, s), 6.83 (1H, dd, J=3.8, 1.8 Hz), 7.46 (1H, app td, J=7.6, 1.3 Hz), 7.53 (1H, d, J=7.8 Hz), 7.59 (1H, app t, J=7.8 Hz), 7.60-7.64 (1H, m), 7.73-7.80 (2H, m), 7.84 (1H, dd, J=7.6, 1.0 Hz), 8.02 (1H, d, J=8.3 Hz), 8.20 (1H, dd, J=1.8, 0.8 Hz), 8.38 (1H, d, J=7.3 Hz)

δ_{C} (125 MHz, DMSO-d₆) δ3.3, 112.7, 114.0, 116.1, 118.2, 120.0, 122.3, 125.1, 126.2, 129.2, 131.1, 132.4, 134.1, 134.2, 139.9, 141.3, 142.7, 146.5, 147.8, 150.8, 156.4, 167.3

mp 177-178° C.

### Example 24

### Preparation of 2′-(((5-(Isoxazole-5″-carbonyl)-5H-[1,2,4]triazino[5,6-b]indol-3-yl)thio)methyl)benzonitrile

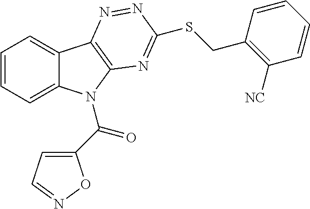 [SMILES: N#Cc1ccccc1CSc1nnc2c3ccccc3n(C(=O)c3ccno3)c2n1]

Following General Procedure 2, to 2′(((5H-[1,2,4]triazino[5,6-b]indol-3-yl)thio)methyl)benzonitrile (100 mg, 0.32 mmol) in DMF (5 mL) was added NaH (13.8 mg, 0.35 mmol) followed by isoxazole-5-carbonyl chloride (62 μL, 0.47 mmol) at 0° C. and the resulting mixture stirred for 16 h. The reaction mixture was quenched with water until a precipitate formed. The precipitate was filtered and dried to give a faint yellow solid (112 mg, 85%).

δ_{H} (500 MHz, DMSO-d, 363K) 4.44 (2H, s), 7.48 (1H, d, J=1.9 Hz), 7.47-7.51 (1H, m), 7.60-7.67 (2H, m), 7.69 (1H, app t, J=7.6 Hz), 7.83-7.87 (2H, m), 8.32 (1H, d, J=8.6 Hz), 8.42 (1H, d, J=7.6 Hz), 8.91 (1H, d, J=1.9 Hz)

δ_{C} (125 MHz, DMSO-d₆) δ2.4, 110.7, 111.9, 116.3, 117.5, 122.5, 126.4, 128.5, 130.3, 132.0, 133.1, 133.4, 138.6, 140.1, 141.2, 142.3, 147.2, 151.9, 155.5, 160.5, 166.5

mp 195-196° C.

### Example 25

### Preparation of 2′-(((5-(Benzoyl-5H-[1,2,4]triazino[5,6-b]indol-3-yl)thio)methyl)benzonitrile

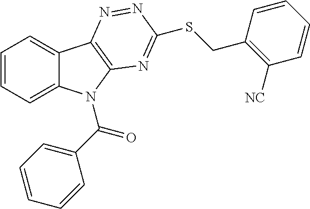 [SMILES: N#Cc1ccccc1CSc1nnc2c3ccccc3n(C(=O)c3ccccc3)c2n1]

Following General Procedure 2, to 2′(((5H-[1,2,4]triazino[5,6-b]indol-3-yl)thio)methyl)benzonitrile (100 mg, 0.32 mmol) in DMF (5 mL) was added NaH (13.8 mg, 0.52 mmol) followed by benzoyl chloride (55 μL, 0.47 mmol) at 0° C. and the resulting mixture stirred for 16 h. The reaction mixture was quenched with water until a precipitate formed. The precipitate was filtered and dried to give a faint yellow solid (97 mg, 72%).

δ_{H} (500 MHz, DMSO-d, 363K) 4.06 (2H, s), 7.34 (1H, d, J=7.9 Hz), 7.47 (1H, app t, J=7.6 Hz), 7.52 (2H, app t, J=7.3 Hz), 7.60 (1H, app t, J=7.9 Hz), 7.63 (1H, app t, J=7.3 Hz), 7.66 (1H, app t, J=7.6 Hz), 7.82-7.86 (2H, m), 7.94 (2H, d, J=7.9 Hz), 8.30 (1H, d, J=8.5 Hz), 8.44 (1H, d, J=7.7 Hz)

δ_{C} (125 MHz, DMSO-d₆) δ1.8, 111.7, 116.2, 117.2, 119.7, 121.3, 125.6, 128.3, 128.4, 130.0, 130.3, 131.6, 133.2, 133.2, 133.3, 133.9, 139.4, 140.2, 142.1, 147.2, 166.4, 167.8

mp 201-202° C.

### Example 26

### Preparation of methyl 4″-(3-((2′-cyanobenzyl)thio)-5H-[1,2,4]triazino[5,6-b]indol-5-yl)-4″-oxobutanoate

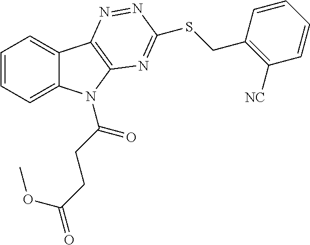 [SMILES: COC(=O)CCC(=O)n1c2ccccc2c2nnc(SCc3ccccc3C#N)nc21]

Following General Procedure 2, to 2′(((5H-[1,2,4]triazino[5,6-b]indol-3-yl)thio)methyl)benzonitrile (150 mg, 0.47 mmol) in DMF (5 mL) was added NaH (20.8 mg, 0.52 mmol) followed by methyl-5-chloro-5-oxopentoate (87 μL, 0.71 mmol) at 0° C. and the resulting mixture stirred for 16 h. The reaction mixture was quenched with water until a precipitate formed. The precipitate was filtered and dried to give a faint yellow solid (165 mg, 81%).

δ_{H} (500 MHz, DMSO-d₆, 363K) 2.80 (2H, t, J=6.3 Hz), 3.62 (3H, s), 3.69 (2H, t, J=6.3 Hz), 4.82 (2H, s), 7.51 (1H, app t, J=7.7 Hz), 7.62 (1H, app t, J=7.6 Hz), 7.70 (1H, app t, J=7.6 Hz), 7.80 (1H, app t, J=7.6 Hz), 7.83 (1H, d, J=7.9 Hz), 7.90 (1H, d, J=7.9 Hz), 8.38 (1H, d, J=7.6 Hz), 8.56 (1H, d, J=8.5 Hz)

δ_{C} (125 MHz, DMSO-d₆) 27.9, 32.9, 34.2, 51.5, 111.9, 117.2, 117.4, 119.4, 121.2, 125.8, 128.4, 130.2, 132.1, 133.2, 133.5, 139.2, 140.5, 142.5, 147.3, 166.5, 172.3, 172.4

mp 144-145° C.

### Example 27

### Preparation of methyl 5″-(3-((2′-cyanobenzyl)thio)-5H-[1,2,4]triazino[5,6-b]indol-5-yl)-5″-oxopentanoate

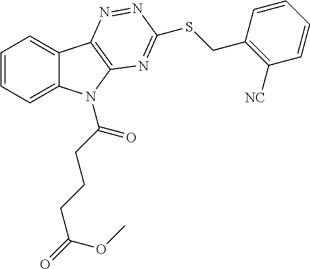 [SMILES: COC(=O)CCCC(=O)n1c2ccccc2c2nnc(SCc3ccccc3C#N)nc21]

Following General Procedure 2, to 2′(((5H-[1,2,4]triazino[5,6-b]indol-3-yl)thio)methyl)benzonitrile (150 mg, 0.47 mmol) in DMF (5 mL) was added NaH (20.8 mg, 0.52 mmol) followed by methyl-5-chloro-5-oxopentoate (98 μL, 0.71 mmol) at 0° C. and the resulting mixture stirred for 16 h. The reaction mixture was quenched with water until a precipitate formed. The precipitate was filtered and dried to give a faint yellow solid (170 mg, 81%).

δ_{H} (500 MHz, DMSO-d₆, 363K) 1.96-2.04 (2H, m) 2.45 (2H, t, J=7.4 Hz), 3.48 (2H, t, J=7.4 Hz), 3.55 (3H, s), 4.80 (2H, s), 7.52 (1H, app t, J=7.7 Hz), 7.61 (1H, app t, J=7.6 Hz), 7.68-7.72 (1H, m), 7.77-7.81 (1H, m), 7.81 (1H, d, J=7.6 Hz), 7.91 (1H, d, J=7.9 Hz), 8.36 (1H, d, J=7.6 Hz), 8.58 (1H, d, J=8.2 Hz)

δ_{C} (125 MHz, DMSO-d₆) 19.0, 32.3, 32.8, 37.9 51.2, 112.0, 117.3, 117.4, 119.3, 121.1, 125.7, 128.5, 130.3, 132.1, 133.2, 133.6, 139.3, 140.4, 142.4, 147.1, 166.6, 172.9, 173.0

mp 166-167° C.

### Example 28

### Preparation of 3-(butylthio)-5H-[1,2,4]triazino[5,6-b]indole

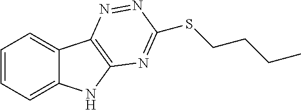 [SMILES: CCCCSc1nnc2c(n1)[nH]c1ccccc12]

To a suspension of 5H-[1,2,4]triazino[5,6-b]indole-3-thiol (80 mg, 0.40 mmol) and CsCO₃ (194 mg, 0.94 mmol) in methanol was added iodobutane (45 μL, 0.4 mmol) and the resulting mixture stirred for 16 h at 80° C. to afford a yellow solution. Upon cooling, the solvents were removed in vacuo and resulting solid was filtered and washed with Et₃N/H₂O mixture to yield a yellow solid (82 mg, 80%).

δ_{H} (400 MHz, DMSO-d₆, 363K) 0.94 (3H, t, J=7.3 Hz), 1.42-1.53 (2H, m), 1.71-1.77 (2H, m), 3.28 (2H, t, J=7.7 Hz), 7.44 (1H, app t, J=7.9 Hz) 7.57 (1H, d, J=8.2 Hz) 7.66-7.72 (1H, m), 8.30 (1H, d, J=7.9 Hz)

δ_{C} (100 MHz, DMSO-d₆) 13.5, 21.5, 29.6, 30.9, 112.7, 117.7, 121.4 122.4, 130.7, 140.3, 140.9, 146.7, 167.3

mp 249-252° C.

### Example 29

### Preparation of 2′(((5-Acetyl-5H-[1,2,4]triazino[5,6-b]indol-3-yl)thio)methyl)benzonitrile

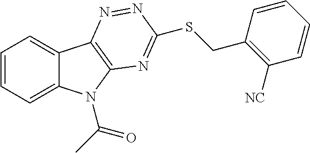 [SMILES: CC(=O)n1c2ccccc2c2nnc(SCc3ccccc3C#N)nc21]

Following General Procedure 2, to 2′(((5H-[1,2,4]triazino[5,6-b]indol-3-yl)thio)methyl)benzonitrile (100 mg, 0.32 mmol) in DMF (5 mL) was added NaH (13.8 mg, 0.35 mmol) followed by acetyl chloride (33.8 μL, 0.47 mmol) at 0° C. and the resulting mixture stirred for 16 h. The reaction mixture was quenched with water until a precipitate formed. The precipitate was filtered and dried to give a faint yellow solid (89 mg, 77%).

δ_{H} (500 MHz, DMSO-d₆, 363K) 2.94 (3H, s), 4.82 (2H, s), 7.51 (1H, app t, J=7.6 Hz), 7.63 (1H, app t, J=7.6 Hz), 7.68-7.72 (1H, m), 7.79-7.83 (1H, m), 7.83 (1H, d, J=7.9 Hz), 7.90 (1H, d, J=6.9 Hz), 8.38 (1H, d, J=7.9 Hz), 8.58 (1H, d, J=8.5 Hz)

δ_{C} (125 MHz, DMSO-d₆) 27.4, 32.8, 112.0, 117.2, 117.4, 119.3, 121.2, 125.7, 128.4, 130.2, 132.1, 133.2, 133.5, 139.3, 140.5, 142.5, 147.3, 166.6, 170.3

mp 207-209° C.

### Example 30

### Preparation of 2′-(((5-Ethyl-5H-[1,2,4]triazino[5,6-b]indol-3-yl)thio)methyl)benzonitrile

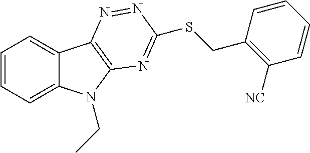 [SMILES: CCn1c2ccccc2c2nnc(SCc3ccccc3C#N)nc21]

Following General Procedure 2, 2′(((5H-[1,2,4]triazino[5,6-b]indol-3-yl)thio)methyl)benzonitrile (100 mg, 0.32 mmol) was added to DMF (5 mL) and cooled to 0° C. NaH (60%, 13.8 mg, 0.34 mmol) was added to the cooled solution and stirred at 0° C. for 10 min. Ethyl iodide (38 μL, 0.47 mmol) was added and reaction mixture was allowed to warm to rt and stirred for 16 h. The reaction mixture was quenched by dropwise addition of water until a precipitate was formed, this was collected by filtration and dried to yield a white powder (85 mg, 78%).

δ_{H} (500 MHz, DMSO-d₆, 363K) 1.34 (3H, t, J=7.3 Hz), 4.45 (2H, q, J=7.3 Hz), 4.77 (2H, s), 7.45-7.48 (1H, m), 7.49 (1H, app t, J=7.6 Hz), 7.65-7.69 (1H, m), 7.78 (1H, app t, J=7.6 Hz), 7.82-7.88 (3H, m), 8.34 (1H, d, J=7.6 Hz)

δ_{C} (125 MHz, DMSO-d₆) 13.4, 32.4, 36.0, 111.2, 112.0, 117.4, 117.6, 121.7, 122.9, 128.1, 130.2, 131.1, 133.0, 133.3, 140.5, 141.1, 141.6, 145.5, 165.9

mp 187-189° C.

### Example 31

### Preparation of 2′-(((5-Butyl-5H-[1,2,4]triazino[5,6-b]indol-3-yl)thio)methyl)benzonitrile

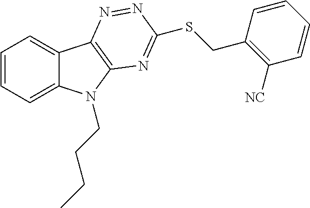 [SMILES: CCCCn1c2ccccc2c2nnc(SCc3ccccc3C#N)nc21]

Following General Procedure 2, 2′(((5H-[1,2,4]triazino[5,6-b]indol-3-yl)thio)methyl)benzonitrile (100 mg, 0.32 mmol), was added to DMF (5 mL) and cooled to 0° C. To the cooled solution was added NaH (60%, 13.8 mg, 0.35 mmol) and stirred at 0° C. for 10 mins. Iodobutane (54 μL, 0.47 mmol) was added and reaction mixture was stirred for 16 h at rt, quenched and the resulting precipitate was filtered and dried to yield a white powder (79 mg, 68%).

δ_{H} (500 MHz, DMSO-d₆, 363K) 0.86 (3H, t, J=7.6 Hz), 1.22-1.30 (2H, m), 1.70-1.76 (2H, m), 4.38 (2H, t, J=7.6 Hz), 4.76 (2H, s), 7.47 (1H, app t, J=7.9 Hz), 7.48 (1H, app t, J=7.6 Hz), 7.64-7.68 (1H, m), 7.75 (1H, app t, J=7.9 Hz), 7.81 (1H, d, J=8.2 Hz), 7.84 (1H, d, J=7.6 Hz), 7.87 (1H, dd, J=7.6, 1.3 Hz), 8.33 (1H, d, J=7.6 Hz)

δ_{C} (125 MHz, DMSO-d₆) 13.6, 19.5, 29.9, 32.4, 40.8, 111.4, 111.9, 117.3, 117.6, 121.6, 122.9, 128.1, 130.0, 131.0, 133.0, 133.3, 140.8, 140.9, 141.5, 145.9, 165.9

mp 159-161° C.

### Example 32

### Preparation of 2′-(((5H-[1,2,4]Triazino[5,6-b]indol-3-yl)sulfinyl)methyl)benzonitrile

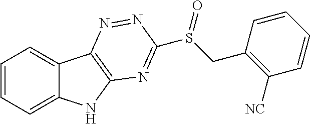 [SMILES: N#Cc1ccccc1CS(=O)c1nnc2c(n1)[nH]c1ccccc12]

Following General Procedure 2, 2′(((5H-[1,2,4]triazino[5,6-b]indol-3-yl)thio)methyl)benzonitrile (50 mg, 0.16 mmol) was added to a solution of TFA (5 mL) and peroxytrifluoroacetic acid (4 M, 40 μL, 0.16 mmol) and stirred for 16 h. The resulting solution was concentrated in vacuo to yield the sulfoxide as a yellow solid (50 mg, 95%)

δ_{H} (500 MHz, DMSO-d₆, 363K) 4.64 (1H, d, J=13.2 Hz), 4.81 (1H, d, J=13.2 Hz), 7.43 (1H, d, J=7.6 Hz), 7.50-7.54 (1H, m), 7.55 (1H, app t, J=7.6 Hz), 7.63-7.67 (1H, m), 7.72 (1H, d, J=8.2 Hz), 7.80 (1H, dd, J=7.9, 1.0 Hz), 7.83-7.86 (1H, m), 8.48 (1H, d, J=7.9 Hz), 13.19 (1H, br s)

δ_{C} (125 MHz, DMSO-d₆) δ7.7, 112.9, 113.2, 117.0, 117.2, 122.6, 123.1, 129.0, 132.1, 132.5, 133.1, 133.1, 133.2, 141.7, 144.5, 147.1, 166.5

mp 237-239° C.§

### Example 33

### Preparation of 3-((4′-Nitrobenzyl)thio)-5H-[1,2,4]triazino[5,6-b]indole

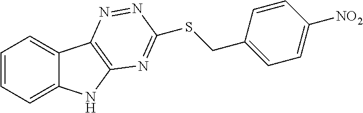 [SMILES: O=[N+]([O-])c1ccc(CSc2nnc3c(n2)[nH]c2ccccc23)cc1]

Following General Procedure 1, 5H-[1,2,4]triazino[5,6-b]indole-3-thiol (150 mg, 0.74 mmol), 4-nitrobenzyl bromide (160 mg, 0.74 mmol) and Et₃N (155 μL, 1.11 mmol) in methanol (15 mL) were reacted for 16 h. The resulting precipitate was filtered and washed with Et₃N/H₂O to give a light yellow solid (97 mg, 62%)

δ_{H} (500 MHz, DMSO-d₆, 363K) 4.69 (2H, s), 7.43 (1H, app t, J=7.6 Hz), 7.58 (1H, d, J=8.2 Hz), 7.68-7.72 (1H, m), 7.81 (2H, d, J=8.8 Hz), 8.18 (2H, d, J=8.8 Hz), 8.30 (1H, d, J=7.9 Hz), 12.66 (1H, s)

δ_{C} (125 MHz, DMSO-d₆) δ3.6, 113.1, 117.9, 121.9, 122.9, 123.8, 130.6, 131.4, 140.7, 141.7, 146.7, 146.9, 146.9, 166.3

mp 235-237° C.§

### Example 34

### Preparation of 2′-(((5-(Phenylsulfonyl)-5H-[1,2,4]triazino[5,6-b]indol-3-yl)thio)methyl)benzonitrile

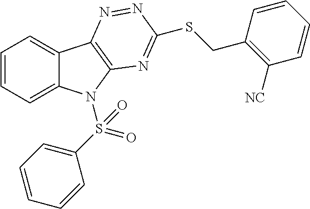 [SMILES: N#Cc1ccccc1CSc1nnc2c3ccccc3n(S(=O)(=O)c3ccccc3)c2n1]

Following General Procedure 2, 2′(((5H-[1,2,4]triazino[5,6-b]indol-3-yl)thio)methyl)benzonitrile (100 mg, 0.32 mmol) was added to DMF (5 mL), cooled to 0° C. followed by the addition of NaH (60%, 13.8 mg, 0.35 mmol) and stirred at 0° C. for 10 mins. Phenyl sulfonyl chloride (40 μL, 0.32 mmol) was added, stirred for 16 h and quenched with water. The resulting precipitate was filtered and dried to yield the product as a white solid (121 mg, 84%).

δ_{H} (500 MHz, DMSO-d₆, 363K) 4.81 (2H, s), 7.52 (1H, td, J=7.6, 1.0 Hz), 7.63 (1H, app t, J=7.6 Hz), 7.63 (2H, td, J=8.8, 1.0 Hz), 7.67-7.71 (1H, m), 7.78 (1H, tt, J=8.8, 1.0 Hz), 7.83-7.86 (1H, m), 7.89 (1H, d, J=7.9), 7.91 (1H, dd, J=7.9, 1.0 Hz), 8.10 (2H, dd, J=8.8, 1.0 Hz), 8.33 (1H, d, J=8.5 Hz), 8.37 (1H, d, J=7.6 Hz)

δ_{C} (125 MHz, DMSO-d₆) δ2.8, 112.0, 114.5, 117.5, 119.3, 121.8, 125.8, 127.4, 128.4, 130.0, 130.2, 132.2, 133.3, 133.4, 135.6, 136.8, 137.9, 140.7, 141.9, 147.1, 167.2

mp 176-177° C.*

### Example 35

### Preparation of 2′-(((5-(Morpholine-4″-carbonyl)-5H-[1,2,4]triazino[5,6-b]indol-3-yl)thio)methyl)benzonitrile

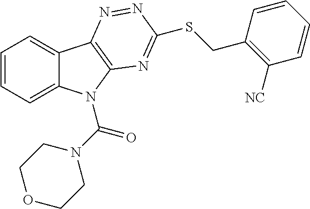 [SMILES: N#Cc1ccccc1CSc1nnc2c3ccccc3n(C(=O)N3CCOCC3)c2n1]

Following General Procedure 2, 2′(((5H-[1,2,4]triazino[5,6-b]indol-3-yl)thio)methyl)benzonitrile (100 mg, 0.32 mmol) was added to DMF (5 mL), cooled to 0° C. followed by the addition of NaH (60%, 13.8 mg, 0.34 mmol) and stirred at 0° C. for 10 mins. Morpholine carbonyl chloride (47 μL, 0.32 mmol) was added, stirred for 16 h, cooled in an ice bath and quenched slowly with dropwise addition of water. The resulting precipitate was filtered and dried to yield the product as a white solid (98 mg, 72%).

δ_{H} (500 MHz, DMSO-d₆, 363K) 3.40-3.79 (8H, m), 4.78 (2H, s), 7.48-7.52 (1H, m), 7.57 (1H, td, J=7.1, 1.9 Hz), 7.66-7.70 (1H, m), 7.76 (1H, d, J=7.6 Hz), 7.77-7.80 (1H, m), 7.83 (1H, d, J=7.6 Hz), 7.89 (1H, dd, J=7.6, 1.3 Hz), 8.37 (1H, d, J=7.6 Hz)

δ_{C} (125 MHz, DMSO-d₆) δ2.7, 44.7, 66.1, 112.0, 113.9, 117.5, 118.5, 121.6, 124.3, 128.3, 130.4, 131.5, 133.2, 133.3, 139.4, 140.9, 141.8, 145.7, 148.4, 166.3

mp 191-193° C.§

### Example 36

### Preparation of 2′-(((5-(Cyclohexylmethyl)-5H-[1,2,4]triazino[5,6-b]indol-3-yl)thio)methyl)benzonitrile

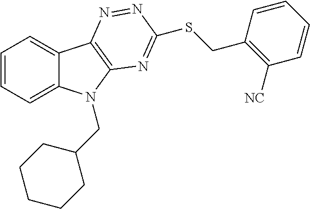 [SMILES: N#Cc1ccccc1CSc1nnc2c3ccccc3n(CC3CCCCC3)c2n1]

Following General Procedure 2, 2′(((5H-[1,2,4]triazino[5,6-b]indol-3-yl)thio)methyl)benzonitrile (100 mg, 0.32 mmol) was added to DMF (5 mL), cooled to 0° C. followed by the addition of NaH (60%, 13.8 mg, 0.34 mmol) and stirred at 0° C. for 10 mins. Bromomethyl cyclohexane (44 μL, 0.32 mmol) was added, stirred for 16 h and quenched with water. The resulting precipitate was filtered and dried to yield the product as a white solid (25 mg, 19%).

δ_{H} (500 MHz, DMSO-d₆, 363K) 0.97-1.12 (6H, m), 1.45-1.50 (2H, m), 1.56-1.63 (2H, m), 1.85-1.92 (1H, m), 4.21 (2H, d, J=7.3 Hz), 4.77 (2H, s), 7.46-7.52 (2H, m), 7.66-7.70 (1H, m), 7.76 (1H, app t, J=7.9 Hz), 7.81-7.87 (2H, m), 7.90 (1H, dd, J=7.6, 1.0 Hz), 8.35 (1H, d, J=7.6 Hz)

δ_{C} (125 MHz, DMSO-d₆) 25.1, 25.6, 30.0, 32.4, 36.6, 46.9, 111.8, 111.9, 117.3, 117.7, 121.5, 122.9, 128.1, 129.8, 131.0, 133.0, 133.4, 140.8, 141.2, 141.6, 146.2, 166.1

mp 201-203° C.§

### Example 37

### Preparation of 2′-(((5-(4″-Fluorobenzyl)-5H-[1,2,4]triazino[5,6-b]indol-3-yl)thio)methyl)benzonitrile

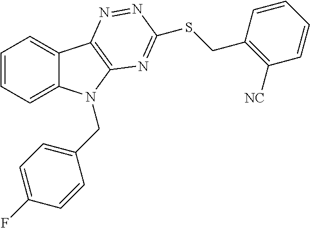 [SMILES: N#Cc1ccccc1CSc1nnc2c3ccccc3n(Cc3ccc(F)cc3)c2n1]

Following General Procedure 2, 2′(((5H-[1,2,4]triazino[5,6-b]indol-3-yl)thio)methyl)benzonitrile (100 mg, 0.32 mmol) was added to DMF (5 mL), cooled to 0° C. followed by the addition of NaH (60%, 13.8 mg, 0.34 mmol) and stirred at 0° C. for 10 mins. 4-Fluorobenzyl bromide (39.3 μL, 0.32 mmol) was added, stirred for 16 h and quenched with water. The resulting precipitate was filtered and dried to yield the product as a white solid (107 mg, 80%).

δ_{H} (500 MHz, DMSO-d₆, 363K) 4.77 (2H, s), 5.66 (2H, s), 7.12 (2H, app t, J=8.8 Hz), 7.34 (2H, m), 7.45 (1H, td, J=7.6, 1.3 Hz), 7.48 (1H, m), 7.57 (1H, td, J=7.6, 1.3 Hz), 7.71-7.74 (2H, m), 7.76-7.88 (2H, m), 8.37 (1H, d, J=7.6 Hz)

δ_{C} (125 MHz, DMSO-d₆) δ2.4, 43.5, 111.6, 111.9, 115.5 (d, J=22 Hz), 115.6, 117.6, 121.7, 123.2, 128.1, 129.5 (d, J=8.6 Hz), 130.2, 131.1, 132.0 (d, J=3.8 Hz), 133.1, 133.3, 140.6, 141.2, 141.5, 146.1, 166.2, 161.6 (d, J=244 Hz)

mp 185-187° C.§

### Example 38

### Preparation of 2′-((5-(2″-Phenylcyclopropanecarbonyl)-5H-[1,2,4]triazino[5,6-b]indol-3-yl thio)methyl)benzonitrile

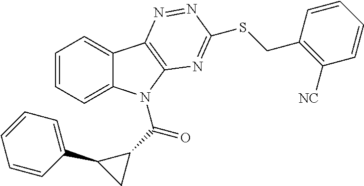 [SMILES: N#Cc1ccccc1CSc1nnc2c3ccccc3n(C(=O)[C@@H]3C[C@H]3c3ccccc3)c2n1]

Trans-2-Phenylcyclopropancarboxylic acid (70 μL, 1.00 mmol) was heated to reflux with thionyl chloride (108 μL, 1.50 mmol) for 1 h. The crude reaction mixture was then concentrated in vacuo to afford 2-phenylcyclopropanecarbonyl chloride.

Following General Procedure 2, NaH (60% in mineral oil, 15 mg, 0.36 mmol) was added to a stirred solution of 2′-(((5H-[1,2,4]triazino[5,6-b]indol-3-yl)thio)methyl)benzonitrile (96 mg, 0.30 mmol) and DMF (2 mL) at 0° C. and the resulting solution was stirred at 0° C. for 15 min. The 2-phenylcyclopropanecarbonyl chloride (70 μL, 1.00 mmol) was then added and the reaction mixture was stirred and allowed to warm to rt over 18 h. The solution was quenched with water (0.7 mL) and the resulting suspension was filtered and dried to yield the title compound as a yellow solid (117 mg, 44%).

δ_{H} (400 MHz, DMSO-d₆) 1.68-1.80 (2H, m), 1.91 (1H, app dt, J=9.4, 4.5 Hz), 2.29-2.36 (1H, m), 4.68 (2H, s), 6.96 (1H, t, J=7.3 Hz), 7.09 (2H, app t, J=7.3 Hz), 7.22 (2H, d, J=7.3 Hz), 7.36-7.47 (2H, m), 7.59-7.70 (2H, m), 7.76-7.83 (2H, m), 8.25 (1H, d, J=8.3 Hz), 8.43 (1H, d, J=8.3 Hz);

δ_{C} (100 MHz, DMSO-d₆) 23.3, 25.7, 28.5, 33.3, 112.7, 113.6, 117.8, 118.2, 118.4, 122.4, 123.5, 127.0, 127.3, 129.1, 129.4, 131.3, 132.0, 133.9, 134.1, 139.4, 142.0, 142.1, 147.3, 166.6, 174.0

mp 139-140° C.

### Example 39

### Preparation of 2′-((5-(2-Cyclopropyl-2-oxoethyl)-5H-[1,2,4]triazino[5,6-b]indol-3-ylthio)methyl)benzonitrile

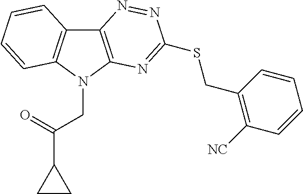 [SMILES: N#Cc1ccccc1CSc1nnc2c3ccccc3n(CC(=O)C3CC3)c2n1]

Following General Procedure 2, NaH (60% in mineral oil), 6 mg, 0.14 mmol) was added to a solution of 2′-(((5H-[1,2,4]triazino[5,6-b]indol-3-yl)thio)methyl)benzonitrile (37 mg, 0.11 mmol) in DMF (1.5 mL) at 0° C. and the resulting mixture was stirred at 0° C. for 15 min. 2-Bromo-1-cyclopropylethanone (28 mg, 0.17 mmol) was then added and the reaction mixture was stirred and allowed to warm to rt over 18 h. The solution was quenched with water (0.4 mL) and the resulting suspension filtered and dried to yield the title compound as a brown solid (16 mg, 38%).

δ_{H} (400 MHz, DMSO-d₆) 1.14-1.26 (4H, m), 3.25-3.31 (1H, m), 4.72 (2H, s), 5.31 (2H, s), 7.52-7.56 (2H, m), 7.71-7.76 (2H, m), 7.81 (1H, d, J=7.6 Hz), 7.86 (1H, d, J=7.6 Hz), 8.33 (1H, d, J=7.6 Hz), 8.43 (1H, d, J=8.1 Hz)

δ_{C} (100 MHz, DMSO-d₆) 10.7, 17.5, 34.3, 69.1, 111.8, 116.6, 117.9, 120.6, 124.3, 126.3, 129.0, 130.8, 133.6, 133.9, 135.1, 141.0, 144.5, 147.9, 167.2, 208.1 (1 peak not observed)

mp 183-184° C.

### Example 40

### Preparation of 2′-((5-(2″-Methylcyclopropanecarbonyl)-5H-[1,2,4]triazino[5,6-b]indol-3-ylthio)methyl)benzonitrile

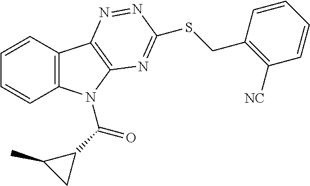

Trans-2-methylcyclopropanecarboxylic acid (103 μL, 1.00 mmol) was heated to reflux with thionyl chloride (108 μL, 1.50 mmol) for 1 h. The crude reaction mixture was then concentrated in vacuo to afford 2-methylcyclopropanecarbonyl chloride.

Following General Procedure 2, NaH (60% in mineral oil, 32 mg, 0.80 mmol) was added to a stirred solution of 2′-(((5H-[1,2,4]triazino[5,6-b]indol-3-yl)thio)methyl)benzonitrile (211 mg, 0.67 mmol) in DMF (2 mL) at 0° C. and the resulting solution was stirred at 0° C. for 15 min. 2-Methylcyclopropanecarbonyl chloride was then added and the reaction mixture was stirred and allowed to warm to rt over 18 h. The solution was quenched with water (1.2 mL) and the resulting suspension filtered and dried to give the title compound as a pale yellow solid (162 mg, 50%).

δ_{H} (400 MHz, DMSO-d₆) 1.05 (1H, app td, J=7.2, 3.7 Hz), 1.19 (3H, d, J=6.0 Hz), 1.46 (1H, app dt, J=8.7, 4.1 Hz), 1.57-1.68 (1H, m), 3.29-3.36 (1H, m), 4.77 (2H, s), 7.50 (1H, app t, J=7.6 Hz), 7.58 (1H, app t, J=7.9 Hz), 7.69 (1H, app t, J=7.6 Hz), 7.74 (1H, app t, J=7.9 Hz), 7.80 (1H, d, J=7.6 Hz), 7.88 (1H, d, J=7.6 Hz), 8.33 (1H, d, J=7.9 Hz), 8.43 (1H, d, J=7.9 Hz)

δ_{C} (100 MHz, DMSO-d₆) 18.5, 19.7, 22.0, 25.3, 33.6, 112.8, 117.8, 118.3, 119.9, 122.0, 126.4, 129.3, 131.2, 132.8, 134.1, 134.4, 140.2, 141.2, 143.4, 148.3, 167.2, 174.2

mp 139-140° C.

### Example 41

### Preparation of 2′-((5-(Cyclopropylmethyl)-5H-[1,2,4]triazino[5,6-b]indol-3-ylthio)methyl)benzonitrile

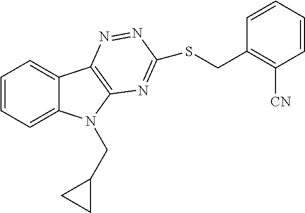 [SMILES: N#Cc1ccccc1CSc1nnc2c3ccccc3n(CC3CC3)c2n1]

Following General Procedure 2, NaH (60% in mineral oil, 15 mg, 0.38 mmol) was added to a solution of 2′-(((5H-[1,2,4]triazino[5,6-b]indol-3-yl)thio)methyl)benzonitrile (100 mg, 0.32 mmol) in DMF (5 mL) at 0° C. and stirred at 0° C. for 15 min. Bromomethylcyclopropane (46 μL, 0.47 mmol) and tetrabutylammonium iodide (23 mg, 0.06 mmol) were then added and the stirring reaction mixture was allowed to warm to rt over 18 h. The solution was quenched with water (0.7 ml) and the resulting suspension filtered and washed with MeOH to give the title compound as a pale yellow solid (62 mg, 52%).

δ_{H} (500 MHz, DMSO-d₆) 0.37-0.50 (4H, m), 1.19-1.33 (1H, m), 4.32 (2H, d, J=6.9 Hz), 4.73 (2H, s), 7.45-7.50 (2H, m), 7.67 (1H, app t, J=7.8 Hz), 7.77 (1H, app t, J=7.6 Hz), 7.8 (1H, d, J=7.8 Hz), 7.81-7.99 (2H, m), 8.34 (1H, d, J=7.8 Hz)

δ_{C} (125 MHz, DMSO-d₆) 3.8, 10.2, 32.4, 45.2, 111.6, 111.9, 117.3, 117.6, 121.6, 122.9, 128.1, 130.0, 131.0, 133.0, 133.4, 140.9, 141.0, 141.5, 145.8, 166.0

mp 149-150° C.

### Example 42

### Preparation of 2′-(((5-(Cyclopropanecarbonyl)-5H-[1,2,4]triazino[5,6-b]indole-3-yl)thio)methyl)benzonitrile

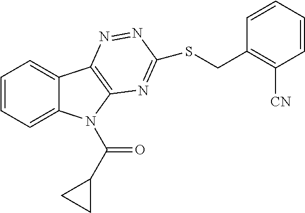 [SMILES: N#Cc1ccccc1CSc1nnc2c3ccccc3n(C(=O)C3CC3)c2n1]

Following General Procedure 2, NaH (60% in mineral oil, 19 mg, 0.48 mmol) was added to a stirred solution of 2′-(((5H-[1,2,4]triazino[5,6-b]indol-3-yl)thio)methyl)benzonitrile (100 mg, 0.32 mmol) in DMF (2 mL) at 0° C. and the resulting solution stirred at this temperature for 15 min. Cyclopropane carbonyl chloride (43 μL, 0.48 mmol) was then added and the reaction mixture was stirred and allowed to warm to rt over 18 h. The solution was quenched with water (1.2 mL) and the resulting suspension filtered and dried to give the title compound as a yellow solid (83 mg, 67%);

δ_{H} (400 MHz, DMSO-d₆) 1.16-1.28 (4H, m), 3.50-3.56 (1H, m), 4.78 (2H, s), 7.48-7.52 (1H, m), 7.58 (1H, app t, J=7.7 Hz), 7.67-7.71 (1H, m), 7.74 (1H, app t, J=7.9 Hz), 7.80 (1H, d, J=7.9 Hz), 7.89 (1H, dd, J=7.7, 1.2 Hz), 8.33 (1H, d, J=7.7 Hz), 8.46 (1H, d, J=7.9 Hz)

δ_{C} (125 MHz, DMSO-d₆) 11.6, 16.1, 32.8, 111.8, 117.1, 117.4, 119.1, 121.1, 125.6, 128.4, 130.0, 130.5, 131.9, 133.2, 133.5, 139.3, 142.5, 147.4, 166.4, 174.0

mp 183-184° C.

### Example 43

### Preparation of 2′-(((9-Bromo-5-(cyclopropylmethyl)-5H-[1,2,4]triazino[5,6-b]indol-3-yl)thio)methyl)benzonitrile

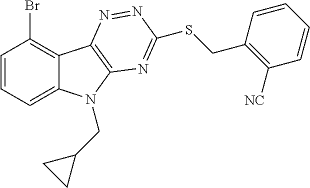 [SMILES: N#Cc1ccccc1CSc1nnc2c3c(Br)cccc3n(CC3CC3)c2n1]

Following General Procedure 2, to 2′-(((9-bromo-5H-[1,2,4]triazino[5,6-b]indol-3-yl)thio)methyl)benzonitrile (100 mg, 0.25 mmol) in DMF (5 mL) was added NaH (12.0 mg, 0.30 mmol) followed by addition of bromomethyl cyclopropane (37 μL, 0.38 mmol) at 0° C. and the resulting mixture stirred for 16 h. The reaction mixture was quenched with water until a precipitate formed. The precipitate was filtered and dried to give a pale yellow solid (57 mg, 85%).

δ_{H} (500 MHz, DMSO-d₆, 363K) 0.40-0.47 (2H, m), 1.24-1.34 (2H, m), 1.54-1.59 (1H, m), 4.32 (2H, d, J=6.9 Hz), 4.79 (2H, s), 7.48 (1H, app t, J=7.6 Hz), 7.57-7.62 (3H, m), 7.67-7.60 (1H, m), 7.84 (1H, d, J=7.8 Hz), 7.88 (1H, d, J=7.8 Hz)

δ_{C} (125 MHz, DMSO-d₆) 3.8, 10.1, 35.8, 54.9, 112.0, 112.3, 116.0, 116.2, 117.5, 128.4, 130.4, 133.1, 133.2, 133.3, 133.4, 141.2, 141.3, 142.1, 145.8, 166.4

mp 159-161° C.

### Example 44

### Preparation of 2′-(((9-Bromo-5-(cyclopropanecarbonyl)-5H-[1,2,4]triazino[5,6-b]indol-3-yl)thio)methyl)benzonitrile

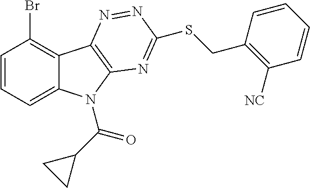 [SMILES: N#Cc1ccccc1CSc1nnc2c3c(Br)cccc3n(C(=O)C3CC3)c2n1]

Following General Procedure 2, to 2′-(((9-bromo-5H-[1,2,4]triazino[5,6-b]indol-3-yl)thio)methyl)benzonitrile (100 mg, 0.25 mmol) in DMF (5 mL) was added NaH (12.0 mg, 0.30 mmol) followed by addition of cyclopropanecarbonyl chloride (40 μL, 0.38 mmol) at 0° C. and the resulting mixture stirred for 16 h. The reaction mixture was quenched with water until a precipitate formed. The precipitate was filtered and dried to give a pale yellow solid (96 mg, 82%).

δ_{H} (500 MHz, DMSO-d₆, 363K) 1.22-1.28 (4H, m), 3.50-3.54 (1H, m), 4.82 (2H, s), 7.50-7.54 (1H, m), 7.66 (1H, app t, J=8.2 Hz), 7.69-7.73 (1H, m), 7.80-7.84 (2H, m), 7.91 (1H, dd, J=7.6, 1.3 Hz), 8.51 (1H, J=8.2 Hz)

δ_{C} (125 MHz, DMSO-d₆) 11.9, 32.8, 54.9, 111.8, 115.8, 116.0, 117.4, 118.6, 128.4, 129.5, 130.1, 132.5, 133.3, 133.6, 140.4, 140.5, 142.4, 147.4, 166.6, 174.0

mp 173-174° C.

### Example 45

### Preparation of methyl-4″-(9-bromo-3-((2′-cyanobenzyl)thio)-5H-[1,2,4]triazino[5,6-b]indol-5-yl-4″-oxobutyrate

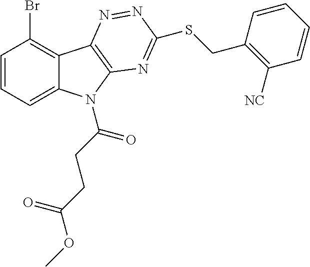 [SMILES: COC(=O)CCC(=O)n1c2cccc(Br)c2c2nnc(SCc3ccccc3C#N)nc21]

Following General Procedure 2, to 2′-(((9-bromo-5H-[1,2,4]triazino[5,6-b]indol-3-yl)thio)methyl)benzonitrile (100 mg, 0.25 mmol) in DMF (5 mL) was added NaH (12.0 mg, 0.30 mmol) followed by addition of methyl-4-chloro-4-oxo butyrate (47 μL, 0.38 mmol) at 0° C. and the resulting mixture stirred for 16 h. The reaction mixture was quenched with water until a precipitate formed. The precipitate was filtered and dried to give a pale yellow solid (126 mg, 97%).

δ_{H} (500 MHz, DMSO-d₆, 363K) 2.80 (2H, t, J=6.3 Hz), 3.62 (3H, s), 3.69 (2H, t, J=6.3 Hz), 4.85 (2H, s), 7.50-7.54 (1H, m), 7.68 (1H, t, J=8.2 Hz), 7.68-7.72 (1H, m), 7.83 (1H, d, J=7.9 Hz), 7.83 (1H, d, J=7.9 Hz), 7.91 (1H, d, J=7.6 Hz), 8.58 (1H, d, J=8.5 Hz)

δ_{C} (125 MHz, DMSO-d₆) 27.9, 32.9, 34.4, 51.6, 111.9, 115.8, 116.1, 117.4, 118.8, 128.5, 129.7, 130.2, 132.7, 133.3, 133.6, 140.3, 140.3, 142.3, 147.1, 166.8, 172.2, 172.4

mp 181-182° C.

### Example 46

### Preparation of 2′-(((5H-[1,2,4]Triazino[5,6-b]indol-3-yl)thio)methyl)benzoic

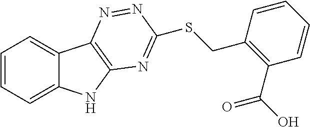 [SMILES: O=C(O)c1ccccc1CSc1nnc2c(n1)[nH]c1ccccc12]

Ethyl 2′-(((5H-[1,2,4]triazino[5,6-b]indol-3-yl)thio)methyl)benzoate (100 mg, 0.27 mmol), was added to a solution of aq. NaOH (1M, 270 μL, 0.27 mmol) in H₂O (5 mL) and stirred at rt for 16 h. The solution was acidified with aq. HCl (1M) until a precipitate was formed, which was filtered and dried in vacuo to yield the final product (92 mg, 99%).

δ_{H} (500 MHz, DMSO-d₆, 363K) 4.99 (2H, s), 7.12-7.22 (3H, m), 7.47-7.52 (3H, m), 7.68-7.72 (1H, m), 8.15 (1H, d, J=7.6 Hz)

δ_{C} (125 MHz, DMSO-d₆) δ2.1, 114.9, 119.1, 119.7, 120.7, 126.0, 127.4, 129.2, 129.5, 129.6, 130.0, 137.2, 141.1, 142.5, 150.4, 166.4, 171.6

mp>300° C.

### Example 47

### Preparation of 3-(((2-(2H-Tetrazol-5-yl)benzyl)thio)-5H-[1,2,4]triazino[5,6-b]indole

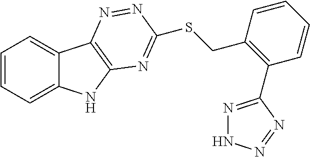 [SMILES: c1ccc(-c2nn[nH]n2)c(CSc2nnc3c(n2)[nH]c2ccccc23)c1]

3-((2′-Cyanobenzyl)thio)-5H-[1,2,4]triazino[5,6-b]indole (100 mg, 0.32 mmol) was added to a solution of NaN₃ (26 mg, 0.40 mmol), NH₄Cl (21 mg, 0.40 mmol) in DMF (5 mL) and heated at 120° C. for 16 h. The resulting solution was acidified using HCl (1M, 10 mL) and extracted with EtOAc (50 mL). The organic layer was washed with water (50 mL), brine (50 mL), dried over MgSO₄, then filtered and left to crystallise for 16 h. The resulting precipitate was filtered and dried to yield the product as a yellow solid (86 mg, 75%).

δ_{H} (500 MHz, DMSO-d₆, 363K) 4.68 (2H, s), 7.37 (1H, app t, J=7.6 Hz), 7.41 (1H, app t, J=7.6 Hz), 7.52 (1H, d, J=8.2 Hz), 7.58-7.66 (2H, m), 7.77 (1H, d, J=7.7 Hz), 7.80 (1H, d, J=7.6 Hz), 8.25 (1H, d, J=7.6 Hz)

δ_{C} (125 MHz, DMSO-d₆) δ2.9, 113.1, 117.9, 121.9, 122.9, 128.6, 130.8, 131.4, 133.5, 133.7, 137.2, 140.8, 141.6, 141.8, 146.9, 163.7, 166.1

mp 272-273° C. (decomp)

### Example 48

### Preparation of 2″-(((9-(o-Tolyl)-5H-[1,2,4]triazino[5,6-b]indol-3-yl)thio)methyl)benzonitrile

### 4-(o-Tolyl)indoline-2,3-dione

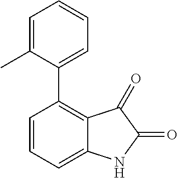 [SMILES: Cc1ccccc1-c1cccc2c1C(=O)C(=O)N2]

Following General Procedure 3,4-bromoisatin (100 mg, 0.44 mmol), potassium 2-methyphenyltrifluoroborate (123 mg, 0.62 mmol), K₃PO₄ (338 mg, 1.59 mmol) and Pd(PPh₃)₂Cl₂ (31 mg, 0.04 mmol) in THF/H₂O (3:1, 3 mL) gave the crude reaction mixture, which was purified via flash column chromatography (eluent 30-40° C. petrol/acetone, 4:1) to afford the product as an orange solid (65 mg, 59%).

δ_{H} (500 MHz, DMSO-d₆) 2.15 (3H, s), 6.93 (2H, m), 7.14 (1H, d, J=7.6 Hz), 7.20-7.26 (1H, m), 7.30 (1H, d, J=7.3 Hz), 7.32-7.37 (1H, m), 7.56 (1H, app t, J=7.9 Hz), 8.84 (1H, br s)

δ_{C} (125 MHz, DMSO-d₆) 19.7, 111.2, 115.7, 125.6, 126.3, 128.6, 128.8, 130.1, 135.7, 136.4, 137.9, 142.8, 149.3, 159.2, 182.0

mp 161-162° C.

### 9-(o-Tolyl)-5H-[1,2,4]triazino[5,6-b]indole-3-thiol

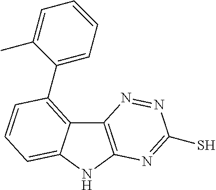 [SMILES: Cc1ccccc1-c1cccc2[nH]c3nc(S)nnc3c12]

Following General Procedure 2,4-(o-tolyl)indoline-2,3-dione (72 mg, 0.30 mmol), thiosemicarbizide (28 mg, 0.30 mmol) and K₂CO₃ (63 mg, 0.45 mmol) in water (6 mL) gave the title compound as a yellow solid (71 mg, 80%).

δ_{H} (500 MHz, DMSO-d₆) 2.26 (3H, s), 7.12 (1H, dd, J=7.8, 0.6 Hz), 7.25-7.28 (2H, m), 7.31-7.34 (2H, m), 7.45 (1H, dd, J=7.8, 0.6 Hz), 7.68 (1H, app t, J=7.8 Hz), 12.52 (1H, br s), 14.27 (1H, s)

δ_{C} (125 MHz, DMSO-d₆) 19.5, 111.7, 115.9, 124.5, 125.7, 128.1, 129.1, 130.0, 131.6, 135.4, 135.7, 137.9, 138.4, 143.1, 149.0, 178.7

mp 297-299° C.

### 2″-(((9-(o-Tolyl)-5H-[1,2,4]triazino[5,6-b]indol-3-yl)thio)methyl)benzonitrile

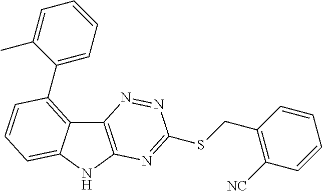 [SMILES: Cc1ccccc1-c1cccc2[nH]c3nc(SCc4ccccc4C#N)nnc3c12]

Following General Procedure 1,9-(o-tolyl)-5H-[1,2,4]triazino[5,6-b]indole-3-thiol (71 mg, 0.30 mmol), Et₃N (63 μL, 0.45 mmol), MeOH (4 mL) and 2-(bromomethyl)benzonitrile (59 mg, 0.30 mmol) gave the title compound as a pale yellow solid (8 mg, 40%).

δ_{H} (500 MHz, DMSO-d₆) 2.06 (3H, s), 4.71 (2H, s), 7.22 (1H, dd, J=7.9, 0.6 Hz), 7.25-7.33 (2H, m), 7.33-7.39 (2H, m), 7.48 (1H, app td, J=7.6, 0.9 Hz), 7.61 (1H, dd, J=7.9, 0.6 Hz), 7.66 (1H, app td, J=7.6, 1.3 Hz), 7.75 (1H, app t, J=7.9 Hz), 7.78 (1H, d, J=7.6 Hz), 7.86 (1H, dd, J=7.6, 1.3 Hz), 12.81 (1H, s)

δ_{C} (125 MHz, DMSO-d₆) 19.6, 32.4, 111.5, 111.9, 115.8, 117.4, 123.9, 125.5, 127.8, 128.3, 129.3, 129.8, 130.4, 130.7, 133.2, 133.4, 135.7, 137.8, 139.0, 140.4, 141.0, 141.6, 146.3, 165.4

mp 217-219° C.

### Example 49

### Preparation of 2″-(((9-(4′-Methoxyphenyl)-5H-[1,2,4]triazino[5,6-b]indol-3-yl)thio)methyl)benzonitrile

### 4-(4′-Methoxyphenyl)indoline-2,3-dione

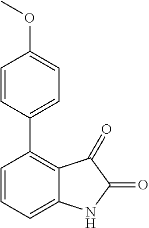 [SMILES: COc1ccc(-c2cccc3c2C(=O)C(=O)N3)cc1]

Following General Procedure 3,4-bromoisatin (100 mg, 0.44 mmol), potassium 4-methoxyphenyltrifluoroborate (133 mg, 0.62 mmol), K₃PO₄ (338 mg, 1.59 mmol) and Pd(PPh₃)₂Cl₂ (31 mg, 0.04 mmol) in THF/H₂O (3:1, 3 mL) gave the crude reaction mixture, which was purified via flash column chromatography (eluent 30-40° C. petrol/acetone, 4:1) to afford the product as an orange solid (71 mg, 63%).

δ_{H} (500 MHz, DMSO-d₆) 3.83 (3H, s), 6.84 (1H, dd, J=7.9, 0.6 Hz), 7.01 (1H, dd, J=7.9, 0.6 Hz), 7.01 (2H, d, J=8.8 Hz), 7.52 (2H, d, J=8.8 Hz), 7.58 (1H, app t, J=7.9 Hz), 11.10 (1H, br s)

δ_{C} (125 MHz, DMSO-d₆) δ5.8, 110.4, 113.5, 115.7, 124.1, 128.5, 130.3, 137.8, 141.5, 151.4, 159.1, 159.8, 183.0

mp 194-195° C.

### 9-(4′-Methoxyphenyl)-5H-[1,2,4]triazino[5,6-b]indole-3-thiol

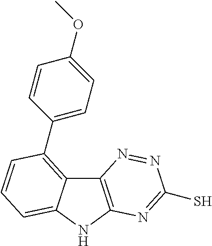 [SMILES: COc1ccc(-c2cccc3[nH]c4nc(S)nnc4c23)cc1]

Following General Procedure 2,4-(4′-methoxyphenyl)indoline-2,3-dione (71 mg, 0.28 mmol), thiosemicarbizide (26 mg, 0.28 mmol) and K₂CO₃ (58 mg, 0.42 mmol) in water (6 mL) gave the title compound as a yellow solid (21 mg, 23%).

δ_{H} (500 MHz, DMSO-d₆) 3.81 (3H, s), 6.91 (1H, dd, J=7.9, 0.6 Hz), 6.96 (1H, dd, J=7.9, 0.6 Hz), 7.00 (2H, d, J=8.6 Hz), 7.41 (1H, app t, J=7.9 Hz), 7.50 (2H, d, J=8.5 Hz), 11.35 (1H, s), 12.63 (1H, s)

δ_{C} (125 MHz, DMSO-d₆) δ5.2, 109.8, 113.0, 113.7, 124.1, 130.1, 130.3, 131.4, 132.2, 137.8, 143.1, 159.2, 162.8, 178.6

mp>300° C.

### 2″-(((9-(4′-Methoxyphenyl)-5H-[1,2,4]triazino[5,6-b]indol-3-yl)thio)methyl)benzonitrile

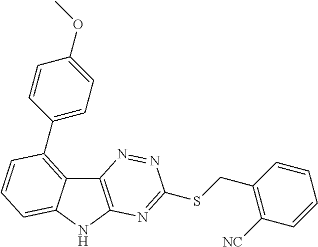 [SMILES: COc1ccc(-c2cccc3[nH]c4nc(SCc5ccccc5C#N)nnc4c23)cc1]

Following General Procedure 1,9-(4′-methoxyphenyl)-5H-[1,2,4]triazino[5,6-b]indole-3-thiol (19 mg, 0.06 mmol), Et₃N (13 μL, 0.09 mmol), MeOH (1.5 mL) and 2-(bromomethyl)benzonitrile (12 mg, 0.06 mmol) gave the title compound as a brown solid (13 mg, 43%).

δ_{H} (500 MHz, DMSO-d₆) 3.88 (3H, s), 4.75 (2H, s), 6.99-7.06 (2H, m), 7.15 (2H, d, J=8.6 Hz), 7.39-7.47 (3H, m), 7.56 (2H, d, J=8.5 Hz), 7.81-7.87 (2H, m), 12.56 (1H, s)

δ_{C} (125 MHz, DMSO-d₆) δ3.1, 55.7, 110.4, 111.8, 113.9, 114.9, 117.5, 124.6, 129.3, 130.7, 130.8, 131.0, 131.8, 132.2, 132.5, 133.0, 139.0, 141.8, 144.8, 150.2, 163.2, 164.6

mp>300° C.

### Example 50

### Preparation of 2″-(((9-(Thiophen-3′-yl)-5H-[1,2,4]triazino[5,6-b]indol-3-yl)thio)methyl)benzonitrile

### 4-(Thiophen-3′-yl)indoline-2,3-dione

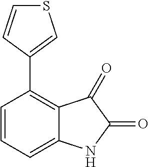 [SMILES: O=C1Nc2cccc(-c3ccsc3)c2C1=O]

Following General Procedure 3,4-bromoisatin (200 mg, 0.88 mmol), potassium 3-thiophenetrifluoroborate (235 mg, 1.24 mmol) K₃PO₄ (675 mg, 3.19 mmol) and Pd(PPh₃)₂Cl₂ (62 mg, 0.09 mmol) in THF/water (3:1, 3 mL) gave the crude reaction mixture, which was purified via flash column chromatography (eluent 30-40° C. petrol/acetone, 4:1) to afford the product as an orange solid (120 mg, 59%).

δ_{H} (500 MHz, DMSO-d₆) 6.85 (1H, dd, J=7.9, 0.6 Hz), 7.20 (1H, dd, J=7.9, 0.6 Hz), 7.50 (1H, dd, J=4.9, 1.3 Hz), 7.58 (1H, app t, J=7.9 Hz), 7.62 (1H, dd, J=4.9, 2.9 Hz), 8.07 (1H, dd, J=2.9, 1.3 Hz), 11.13 (1H, br s)

δ_{C} (125 MHz, DMSO-d₆) 110.8, 113.9, 123.8, 125.6, 126.3, 128.4, 135.8, 137.2, 138.0, 151.6, 158.9, 183.0

mp 213-216° C.

### 9-(Thiophen-3′-yl)-5H-[1,2,4]triazino[5,6-b]indol-3-thiol

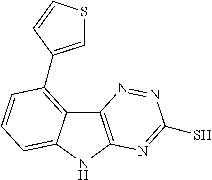 [SMILES: Sc1nnc2c(n1)[nH]c1cccc(-c3ccsc3)c12]

Following General Procedure 2,4-(thiophen-3′-yl)indoline-2,3-dione (137 mg, 0.60 mmol), thiosemicarbizide (54 mg, 0.60 mmol) and K₂CO₃ (124 mg, 0.90 mmol) in water (5 mL) gave the title compound as an orange solid (148 mg, 87%).

δ_{H} (500 MHz, DMSO-d₆) 7.40 (1H, dd, J=7.9, 0.6 Hz), 7.46 (1H, dd, J=7.9, 0.6 Hz), 7.61-7.64 (1H, m), 7.65-7.69 (2H, m), 8.12 (1H, dd, J=3.2, 1.3 Hz), 12.54 (1H, br s), 14.45 (1H, br s)

δ_{C} (125 MHz, DMSO-d₆) 111.6, 114.4, 123.8, 125.3, 125.8, 128.4, 131.8, 133.2, 135.8, 138.6, 143.8, 148.9, 178.5

mp>300° C.

### 2″-(((9-(Thiophen-3′-yl)-5H-[1,2,4]triazino[5,6-b]indol-3-yl)thio)methyl)benzonitrile

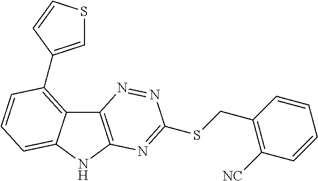 [SMILES: N#Cc1ccccc1CSc1nnc2c(n1)[nH]c1cccc(-c3ccsc3)c12]

Following General Procedure 1, 9-(thiophen-3′-yl)-5H-[1,2,4]triazino[5,6-b]indol-3-thiol (20 mg, 0.07 mmol), Et₃N (15 μL, 0.11 mmol), MeOH (5 mL) and 2-(bromomethyl)benzonitrile (14 mg, 0.07 mmol) gave the title compound as a solid (16 mg, 57%).

δ_{H} (500 MHz, DMSO-d₆) 4.76 (2H, s), 7.39 (1H, d, J=7.9 Hz), 7.49 (1H, app t, J=7.6), 7.55 (1H, d, J=7.9), 7.62 (1H, app t, J=7.6 Hz), 7.64-7.77 (2H, m), 7.77-7.86 (2H, m), 8.12 (1H, d, J=2.9 Hz), 8.46 (1H, d, J=2.9 Hz), 12.85 (1H, br s)

δ_{C} (125 MHz, DMSO-d₆) δ1.6, 111.0, 111.6, 117.0, 117.4, 124.8, 125.7, 125.9, 128.6, 129.8, 130.8, 132.2, 133.7, 133.2, 133.5, 135.0, 139.7, 140.9, 141.2, 143.9, 163.6

mp 274-276° C.

### Example 51-preparation of 2″-(((9-Cyclopropyl-5H-[1,2,4]triazino[5,6-b]indol-3-yl)thio)methyl)benzonitrile

### 4-Cyclopropylindoline-2,3-dione

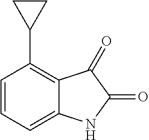 [SMILES: O=C1Nc2cccc(C3CC3)c2C1=O]

Following General Procedure 3,4-bromoisatin (300 mg, 1.33 mmol), potassium cyclopropyltrifluoroborate (275 mg, 1.86 mmol), K₃PO₄ (1.01 g, 14.8 mmol) and Pd(dppf)₂Cl₂ (93 mg, 0.13 mmol) in THF/H₂O (3:1, 4 mL) gave the crude reaction mixture, which was purified via flash column chromatography (eluent 30-40° C. petrol/acetone, 4:1) to afford the product as an orange solid. (36 mg, 43%).

δ_{H} (500 MHz, DMSO-d₆); 0.80-0.84 (2H, m), 1.06-1.15 (2H, m), 2.78-2.89 (1H, m), 6.49 (1H, d, J=7.8 Hz), 6.62 (1H, d, J=7.8 Hz), 7.40 (1H, app t, J=7.8 Hz), 10.98 (1H, br s)

δ_{C} (125 MHz, DMSO-d₆) 10.6, 10.9, 108.4, 116.8, 126.3, 138.2, 147.6, 150.4, 159.2, 176.7

mp 145-148° C.

### 9-Cyclopropyl-5H-[1,2,4]triazino[5,6-b]indole-3-thiol

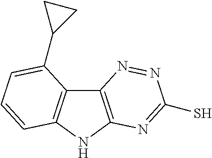 [SMILES: Sc1nnc2c(n1)[nH]c1cccc(C3CC3)c12]

Following General Procedure 2,4-cyclopropylindoline-2,3-dione (48 mg, 0.26 mmol), thiosemicarbizide (24 mg, 0.26 mmol) and K₂CO₃ (53 mg, 0.39 mmol) in water (3 mL) gave the title compound as an orange solid. (30 mg, 51%).

δ_{H} (500 MHz, DMSO-d₆) 0.88-0.93 (2H, m), 1.15-1.20 (2H, m), 2.76-2.80 (1H, m), 6.80 (1H, d, J=7.7 Hz), 7.19 (1H, d, J=7.7 Hz), 7.50 (1H, app t, J=7.8 Hz), 12.37 (1H, br s), 14.53 (1H, br s)

δ_{C} (125 MHz, DMSO-d₆) 11.2, 13.3, 110.3, 117.0, 117.5, 132.2, 132.8, 137.7, 143.6, 149.9, 179.4

mp>300° C.

2″-(((9-Cyclopropyl-5H-[1,2,4]triazino[5,6-b]indol-3-yl)thio)methyl)benzonitrile

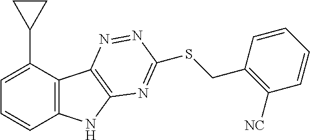 [SMILES: N#Cc1ccccc1CSc1nnc2c(n1)[nH]c1cccc(C3CC3)c12]

Following General Procedure 1,9-cyclopropyl-5H-[1,2,4]triazino[5,6-b]indole-3-thiol (9 mg, 0.04 mmol), Et₃N (8 μL, 0.06 mmol), MeOH (1.5 mL) and 2-(bromomethyl)benzonitrile (7 mg, 0.04 mmol) gave the title compound as a yellow solid (15 mg, 86%).

δ_{H} (500 MHz, DMSO-d₆) 0.94 (2H, dd, J=5.1, 2.0 Hz), 1.18-1.26 (3H, m), 4.74 (2H, s), 6.86 (1H, d, J=7.9 Hz), 7.32 (1H, d, J=7.9 Hz), 7.48 (1H, app t, J=7.6 Hz), 7.55 (1H, app t, J=7.9 Hz), 7.67 (1H, app t, J=7.6 Hz), 7.83 (1H, d, J=7.6 Hz), 7.87 (1H, d, J=7.6 Hz), 12.62 (1H, s)

δ_{C} (125 MHz, DMSO-d₆) 11.1, 18.4, 32.3, 111.3, 111.9, 117.2, 117.6, 121.5, 123.1, 128.1, 130.2, 131.1, 133.0, 133.2, 141.1, 141.3, 141.5, 146.2, 166.0

mp 243-245° C.

### Example 52

### Biological Evaluation

Compounds were evaluated for their CB2R agonist potency in the following assays:

Assay 1—cAMP Assay on hCB2R Clone 3D8.3:

Chinese hamster ovary cells (CHO)-K1 were transfected with hCB2R plasmid using Genejuice (Merck). After 48 h the cells were transferred into flasks in the presence of the antibiotic G418 and expanded (with continuous replenishment of the antibiotic). Two single-cell cloning rounds were then performed, followed by verification of the presence of the tag of the plasmid and confirming the receptor was functional in cAMP studies. For the screening of compounds: Briefly, hCNR2 CHO-K1 (3D8.3 clone) cells were plated in Optimem+1% FCS (30000 cells, 100 ul per well) in half-area 96-well plates (Corning 3885) and placed in an incubator 37° C. 5% CO2 overnight. Next day, the medium was aspirated and replaced with ‘antibody solution’ (22.5 ul) (provided concentrated, diluted 1 in 3 with ‘assay buffer’ provided). The freshly made compounds (at a ×4 final concentration) or solvent alone (1.2% DMSO) were added to the wells and incubated for 30 minutes in a 37° C. incubator 5% CO2. A mixture of: Lysis solution (38%), substrates (Emerald-II™ (10%) and Galacton-Star® (2%)) and enzyme donor-labeled cAMP (50%) was added to the wells and incubated in the dark for one hour at room temperature. 30 μl of detection reagent (enzyme acceptor, provided) was then added to the wells and incubated in the dark for 3 hours at room temperature. The luminescence was then read with a Perkin-Elmer luminometer.

Assay 2—DiscoveRx cAMP Hunter Express GPCR Assay

The cAMP Hunter eXpress GPCR assay kit and cells used are from DiscoveRx Corporation (95-0040E2) using the manufacturer's protocol without the optional IBMX step. CB2R agonists HU-308 or JWH 133 (both from Tocris, stock solutions prepared in DMSO) were used as positive controls for the assay in a range of concentrations from 30 pM to 3 uM. Briefly, hCNR2 CHO-K1 cells were plated in the provided medium (50 ul per well) in half-area 96-well plates (Corning 3885) and placed in an incubator 37° C. 5% CO2 overnight. Next day, the medium was aspirated and replaced with ‘antibody solution’ (22.5 ul) (provided concentrated, diluted 1 in 3 with ‘assay buffer’ provided). The freshly made compounds (at a ×4 final concentration) or solvent alone (1.2% DMSO) were added to the wells and incubated for 30 minutes in a 37° C. incubator 5% CO2. A mixture of lysis solution and two substrates (provided with the DiscoveRx kit) was added to the wells and incubated in the dark for one hour at room temperature. A detection reagent (provided) was then added to the wells and incubated in the dark for 3 hours at room temperature. The luminescence was then read with a Perkin-Elmer luminometer.

The results obtained are shown in Table 1 below:

TABLE 1

| Example | | | Assay 1 | | Assay 2 | |
|---|---|---|---|---|---|---|
| No. | Structure | MW | IC₅₀ | | IC₅₀ | |
| 1 | 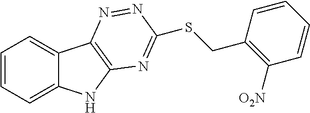 [SMILES: O=[N+]([O-])c1ccccc1CSc1nnc2c(n1)[nH]c1ccccc12] | 337.06 | 129 | nM | 33 | nM |
| 2 | 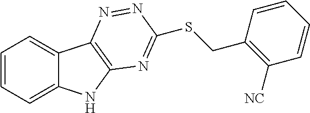 [SMILES: N#Cc1ccccc1CSc1nnc2c(n1)[nH]c1ccccc12] | 317.07 | 160 | nM | 112 | nM |
| 3 | 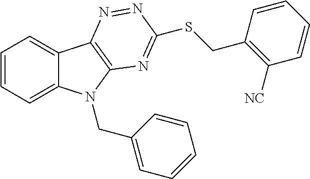 [SMILES: N#Cc1ccccc1CSc1nnc2c3ccccc3n(Cc3ccccc3)c2n1] | 407.12 | 96 | nM | 214 | nM |
| 4 | 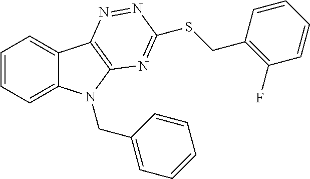 [SMILES: Fc1ccccc1CSc1nnc2c3ccccc3n(Cc3ccccc3)c2n1] | 400.12 | 850 | nM | | |
| 5 | 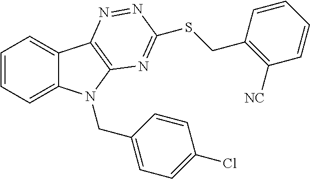 [SMILES: N#Cc1ccccc1CSc1nnc2c3ccccc3n(Cc3ccc(Cl)cc3)c2n1] | 441.08 | about 600 | nM | | |
| 6 | 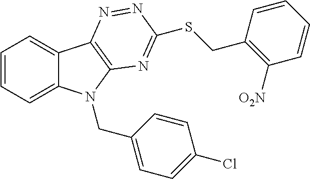 [SMILES: O=[N+]([O-])c1ccccc1CSc1nnc2c3ccccc3n(Cc3ccc(Cl)cc3)c2n1] | 461.07 | 631 | nM | 109 | nM |
| 7 | 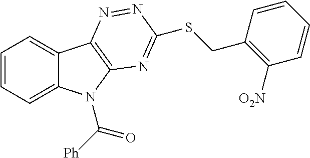 [SMILES: O=C(c1ccccc1)n1c2ccccc2c2nnc(SCc3ccccc3[N+](=O)[O-])nc21] | 441.09 | 840 | nM | 75 | nM |
| 8 | 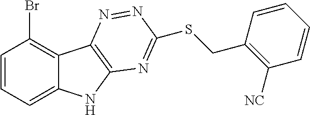 [SMILES: N#Cc1ccccc1CSc1nnc2c(n1)[nH]c1cccc(Br)c12] | 394.98 | 228 | nM | 39 | nM |
| 9 | 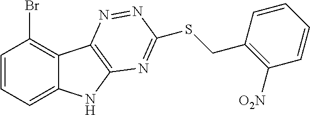 [SMILES: O=[N+]([O-])c1ccccc1CSc1nnc2c(n1)[nH]c1cccc(Br)c12] | 414.98 | 280 | nM | 37 | nM |
| 10 | 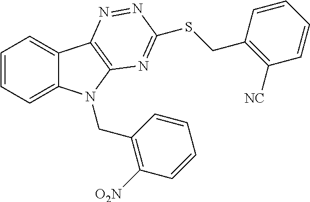 [SMILES: N#Cc1ccccc1CSc1nnc2c3ccccc3n(Cc3ccccc3[N+](=O)[O-])c2n1] | 453.11 | 355 | nM | 53 | nM |
| 11 | 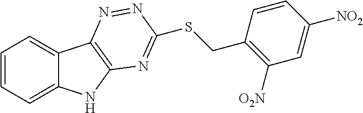 [SMILES: O=[N+]([O-])c1ccc(CSc2nnc3c(n2)[nH]c2ccccc23)c([N+](=O)[O-])c1] | 382.05 | 3.9 | μM | 576 | nM |
| 12 | 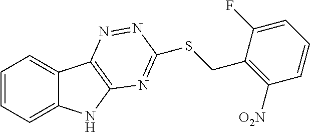 [SMILES: O=[N+]([O-])c1cccc(F)c1CSc1nnc2c(n1)[nH]c1ccccc12] | 355.05 | 1.9 | μM | 539 | nM |
| 13 | 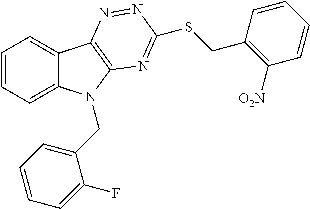 [SMILES: O=[N+]([O-])c1ccccc1CSc1nnc2c3ccccc3n(Cc3ccccc3F)c2n1] | 310.07 | 201 | nM | 201 | nM |
| 14 | 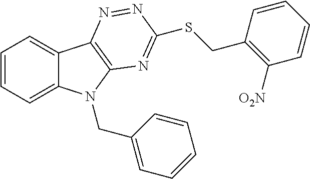 [SMILES: O=[N+]([O-])c1ccccc1CSc1nnc2c3ccccc3n(Cc3ccccc3)c2n1] | 427.11 | ~40% activity at 3 μM | | | |
| 15 | 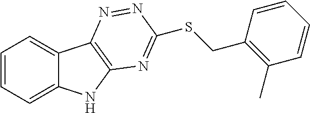 [SMILES: Cc1ccccc1CSc1nnc2c(n1)[nH]c1ccccc12] | 306.09 | 1.9 | pM | 215 | nM |
| 16 | 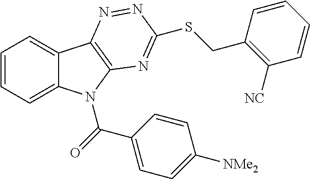 [SMILES: CN(C)c1ccc(C(=O)n2c3ccccc3c3nnc(SCc4ccccc4C#N)nc32)cc1] | 464.14 | ~40% activity at 3 μM | | 20% decrease at 1 and 3 μM | |
| 17 | 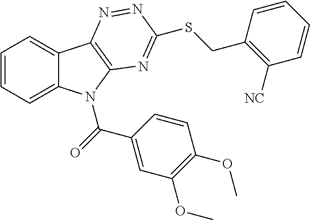 [SMILES: COc1ccc(C(=O)n2c3ccccc3c3nnc(SCc4ccccc4C#N)nc32)cc1OC] | 481.12 | ~60% activity at 3 μM | | | |
| 18 | 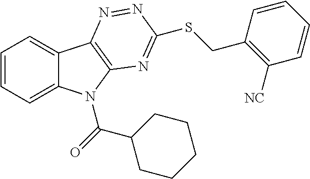 [SMILES: N#Cc1ccccc1CSc1nnc2c3ccccc3n(C(=O)C3CCCCC3)c2n1] | 427.15 | 12 | nM | 32 | nM |
| 19 | 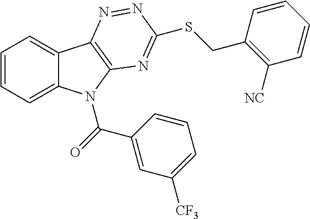 [SMILES: N#Cc1ccccc1CSc1nnc2c3ccccc3n(C(=O)c3cccc(C(F)(F)F)c3)c2n1] | 489.09 | 57 | nM | 27 | nM |
| 20 | 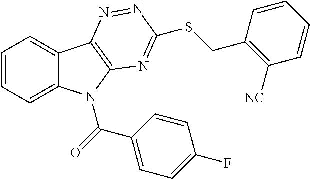 [SMILES: N#Cc1ccccc1CSc1nnc2c3ccccc3n(C(=O)c3ccc(F)cc3)c2n1] | 439.46 | 32 | nM | 57 | nM |
| 21 | 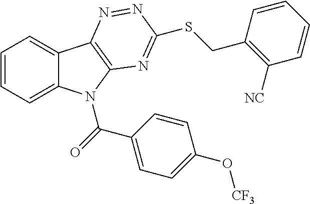 [SMILES: N#Cc1ccccc1CSc1nnc2c3ccccc3n(C(=O)c3ccc(OC(F)(F)F)cc3)c2n1] | 505.47 | 111 | nM | 73 | nM |
| 22 | 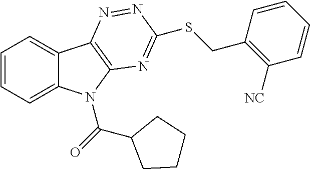 [SMILES: N#Cc1ccccc1CSc1nnc2c3ccccc3n(C(=O)C3CCCC3)c2n1] | 413.13 | 14 | nM | 49 | nM |
| 23 | 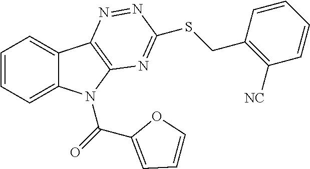 [SMILES: N#Cc1ccccc1CSc1nnc2c3ccccc3n(C(=O)c3ccco3)c2n1] | 411.08 | 31 | nM | 46 | nM |
| 24 | 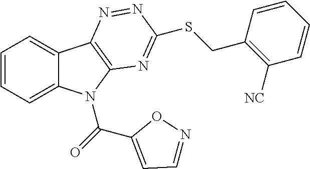 [SMILES: N#Cc1ccccc1CSc1nnc2c3ccccc3n(C(=O)c3ccno3)c2n1] | 412.07 | 14 | nM | 19 | nM |
| 25 | 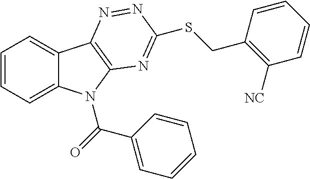 [SMILES: N#Cc1ccccc1CSc1nnc2c3ccccc3n(C(=O)c3ccccc3)c2n1] | 421.10 | 38 | nM | 113 | nM |
| 26 | 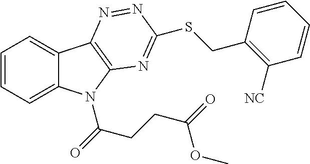 [SMILES: COC(=O)CCC(=O)n1c2ccccc2c2nnc(SCc3ccccc3C#N)nc21] | 431.10 | 43 | nM | 48 | nM |
| 27 | 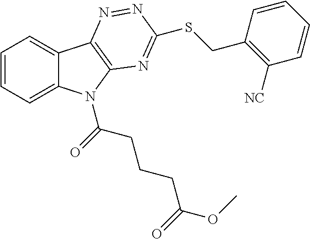 [SMILES: COC(=O)CCCC(=O)n1c2ccccc2c2nnc(SCc3ccccc3C#N)nc21] | 445.12 | 45 | nM | 42 | nM |
| 28 | 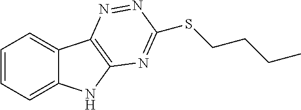 [SMILES: CCCCSc1nnc2c(n1)[nH]c1ccccc12] | 258.09 | 95 | nM | 95 | nM |

| | | | | |
|---|---|---|---|---|
| 29 | 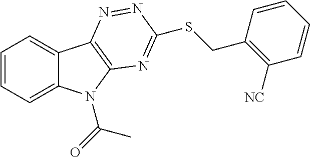 [SMILES: CC(=O)n1c2ccccc2c2nnc(SCc3ccccc3C#N)nc21] | 359.08 | >3 | μM |
| 30 | 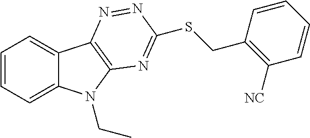 [SMILES: CCn1c2ccccc2c2nnc(SCc3ccccc3C#N)nc21] | 345.10 | 674 | nM |
| 31 | 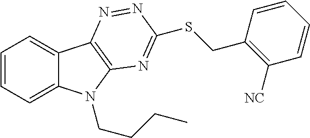 [SMILES: CCCCn1c2ccccc2c2nnc(SCc3ccccc3C#N)nc21] | 373.14 | 106 | nM |
| 32 | 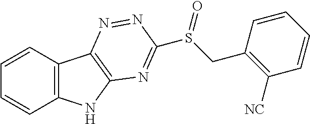 [SMILES: N#Cc1ccccc1CS(=O)c1nnc2c(n1)[nH]c1ccccc12] | 333.07 | 3550 | nM |
| 33 | 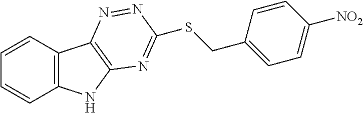 [SMILES: O=[N+]([O-])c1ccc(CSc2nnc3c(n2)[nH]c2ccccc23)cc1] | 337.06 | 617 | nM |
| 34 | 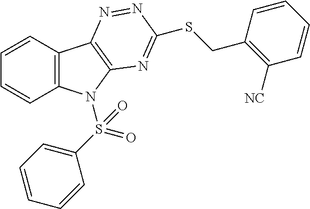 [SMILES: N#Cc1ccccc1CSc1nnc2c3ccccc3n(S(=O)(=O)c3ccccc3)c2n1] | 457.07 | 76 | nM |
| 35 | 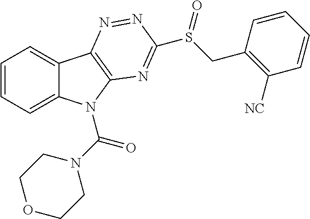 [SMILES: N#Cc1ccccc1CS(=O)c1nnc2c3ccccc3n(C(=O)N3CCOCC3)c2n1] | | 92 | nM |
| 36 | 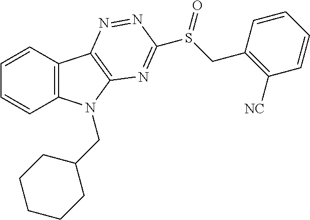 [SMILES: N#Cc1ccccc1CS(=O)c1nnc2c3ccccc3n(CC3CCCCC3)c2n1] | 413.17 | 270 | nM |
| 37 | 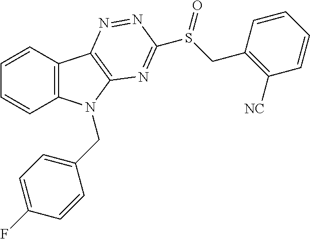 [SMILES: N#Cc1ccccc1CS(=O)c1nnc2c3ccccc3n(Cc3ccc(F)cc3)c2n1] | 425.11 | 45 | nM |
| 38 | 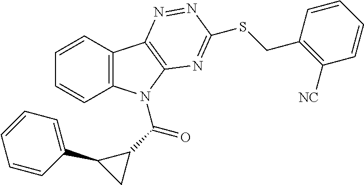 | 461.13 | 72 | nM |
| 39 | 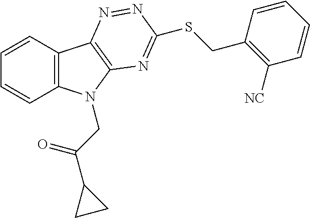 [SMILES: N#Cc1ccccc1CSc1nnc2c3ccccc3n(CC(=O)C3CC3)c2n1] | 399.12 | 549 | nM |
| 40 | 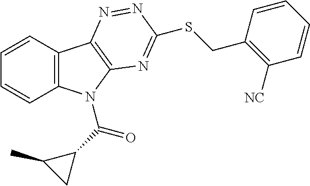 [SMILES: C[C@@H]1C[C@H]1C(=O)n1c2ccccc2c2nnc(SCc3ccccc3C#N)nc21] | 399.12 | 628 | nM |
| 41 | 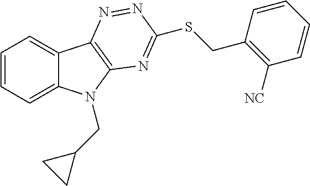 [SMILES: N#Cc1ccccc1CSc1nnc2c3ccccc3n(CC3CC3)c2n1] | 371.12 | 144 | nM |
| 42 | 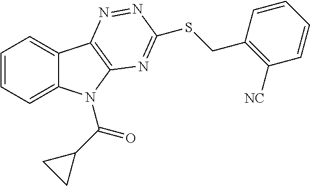 [SMILES: N#Cc1ccccc1CSc1nnc2c3ccccc3n(C(=O)C3CC3)c2n1] | 385.10 | 181 | nM |
| 43 | 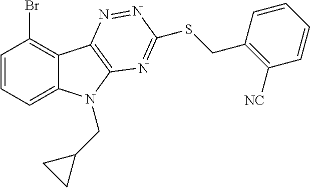 [SMILES: N#Cc1ccccc1CSc1nnc2c3c(Br)cccc3n(CC3CC3)c2n1] | 449.03 | 20 | nM |
| 44 | 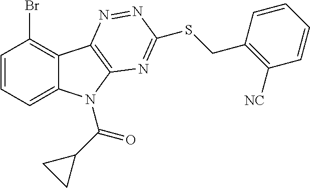 [SMILES: N#Cc1ccccc1CSc1nnc2c3c(Br)cccc3n(C(=O)C3CC3)c2n1] | 463.01 | 16 | nM |
| 45 | 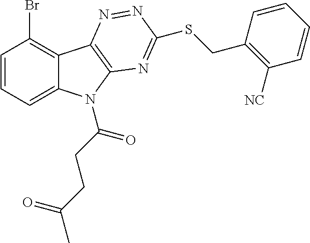 [SMILES: CC(=O)CCC(=O)n1c2cccc(Br)c2c2nnc(SCc3ccccc3C#N)nc21] | 493.02 | 25 | nM |
| 46 | 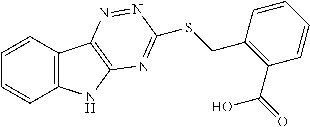 [SMILES: O=C(O)c1ccccc1CSc1nnc2c(n1)[nH]c1ccccc12] | 336.07 | 2185 | nM |
| 47 | 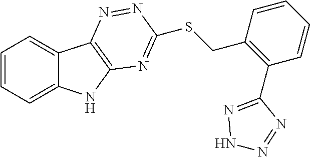 [SMILES: c1ccc(-c2nn[nH]n2)c(CSc2nnc3c(n2)[nH]c2ccccc23)c1] | 360.09 | 11 | nM |
| 48 | 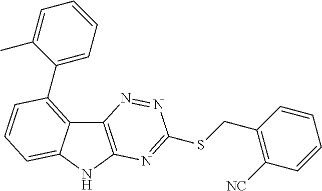 [SMILES: Cc1ccccc1-c1cccc2[nH]c3nc(SCc4ccccc4C#N)nnc3c12] | 407.12 | 4300 | nM |
| 49 | 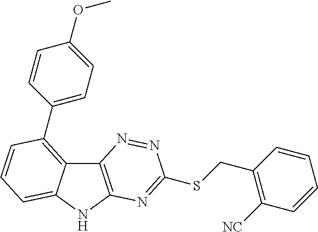 [SMILES: COc1ccc(-c2cccc3[nH]c4nc(SCc5ccccc5C#N)nnc4c23)cc1] | 423.12 | 2620 | nM |
| 50 | 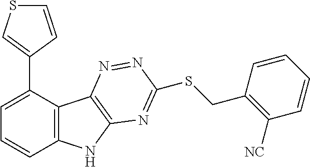 [SMILES: N#Cc1ccccc1CSc1nnc2c(n1)[nH]c1cccc(-c3ccsc3)c12] | 399.06 | 1760 | nM |
| 51 | 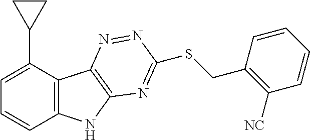 [SMILES: N#Cc1ccccc1CSc1nnc2c(n1)[nH]c1cccc(C3CC3)c12] | 357.1 | 231 | nM |

### REFERENCES

1. Galiegue S, Mary S, March and J, Dussossoy D, Carriere D, Carayon P, et al. Expression of central and peripheral cannabinoid receptors in human immune tissues and leukocyte subpopulations. Eur J Biochem 1995; 232(1):54-61.
2. Chakrabarti A, Onaivi E S, Chaudhuri G. Cloning and sequencing of a cDNA encoding the mouse brain-type cannabinoid receptor protein. DNA Seq 1995; 5(6):385-8.
3. Sugiura T, Kondo S, Kishimoto S, Miyashita T, Nakane S, Kodaka T, et al. Evidence That 2-Arachidonoylglycerol but Not N-Palmitoylethanolamine or Anandamide Is the Physiological Ligand for the Cannabinoid CB2 Receptor. COMPARISON OF THE AGONISTIC ACTIVITIES OF VARIOUS CANNABINOID RECEPTOR LIGANDS 1N HL-60 CELLS. J. Biol. Chem. 2000; 275(1):605-12.
4. Georgieva T, Devanathan S, Stropova D, Park C K, Salamon Z, Tollin G, et al. Unique agonist-bound cannabinoid CB1 receptor conformations indicate agonist specificity in signaling. European Journal of Pharmacology 2008; 581(1-2):19-29.
5. Berdyshev E V, Schmid P C, Krebsbach R J, Schmid H H O. Activation of PAF receptors results in enhanced synthesis of 2-arachidonoylglycerol (2-AG) in immune cells. FASEB J. 2001; 15(12):2171-8.
6. Walter L, Nephi S. Endothelin-1 increases 2-arachidonoyl glycerol (2-AG) production in astrocytes. Glia 2003; 44(1):85-90.
7. Sugiura T, Kishimoto S, Oka S, Gokoh M. Biochemistry, pharmacology and physiology of 2-arachidonoylglycerol, an endogenous cannabinoid receptor ligand. Progress in Lipid Research 2006; 45(5):405-46.
8. Liu J, Batkai S, Pacher P, Harvey-White J, Wagner J A, Cravatt B F, et al. Lipopolysaccharide Induces Anandamide Synthesis in Macrophages via CD14/MAPK/Phosphoinositide 3-Kinase/NF-{kappa}B Independently of Platelet-activating Factor. J. Biol. Chem. 2003; 278(45):45034-9.
9. Walter L, Dinh T, Stella N. ATP Induces a Rapid and Pronounced Increase in 2-Arachidonoylglycerol Production by Astrocytes, a Response Limited by Monoacylglycerol Lipase. J. Neurosci. 2004; 24(37):8068-74.
10. Felder C C, Nielsen A, Briley E M, Palkovits M, Priller J, Axelrod J, et al. Isolation and measurement of the endogenous cannabinoid receptor agonist, anandamide, in brain and peripheral tissues of human and rat. FEBS Letters 1996; 393(2-3):231-5.
11. Palazuelos J, Davoust N, Julien B, Hatterer E, Aguado T, Mechoulam R, et al. The CB2 Cannabinoid Receptor Controls Myeloid Progenitor Trafficking: INVOLVEMENT IN THE PATHOGENESIS OF AN ANIMAL MODEL OF MULTIPLE SCLEROSIS. J. Biol. Chem. 2008; 283(19):13320-9.
12. Karsak M, Gaffal E, Date R, Wang-Eckhardt L, Rehnelt J, Petrosino S, et al. Attenuation of Allergic Contact Dermatitis Through the Endocannabinoid System. Science 2007; 316(5830): 1494-7.
13. Kimball E S, Schneider C R, Wallace N H, Hornby P J. Agonists of cannabinoid receptor 1 and 2 inhibit experimental colitis induced by oil of mustard and by dextran sulfate sodium. Am J Physiol Gastrointest Liver Physiol 2006; 291(2):G364-71.
14. Steffens S, Veillard N R, Arnaud C, Pelli G, Burger F, Staub C, et al. Low dose oral cannabinoid therapy reduces progression of atherosclerosis in mice. Nature 2005; 434(7034):782-6.
15. Montecucco F, Burger F, Mach F, Steffens S. CB2 cannabinoid receptor agonist JWH-015 modulates human monocyte migration through defined intracellular signaling pathways. Am J Physiol Heart Circ Physiol 2008; 294(3):H1145-55.
16. Raborn E, Marciano-Cabral F, Buckley N, Martin B, Cabral G. The Cannabinoid Delta-9-tetrahydrocannabinol Mediates Inhibition of Macrophage Chemotaxis to RANTES/CCL5: Linkage to the CB2 Receptor. Journal of Neuroimmune Pharmacology 2008; 3(2):1 17-29.
17. Sacerdote P, Massi P, Panerai A E, Parolaro D. In vivo and in vitro treatment with the 50 synthetic cannabinoid CP55,940 decreases the in vitro migration of macrophages in the rat: involvement of both CB1 and CB2 receptors. Journal of Neuroimmunology 2000; 109(2):155-63.

1. A method of treating a disease or condition in which the stimulation of CB2R is beneficial, the method comprising administering a therapeutically effective amount of a compound of formula I: 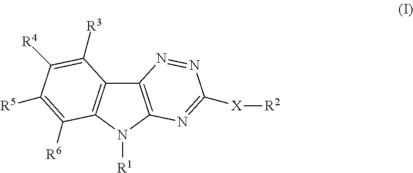
wherein:
X is —S—, —O—, —SO—, NR^{a} or —CH₂—;
R^{a} is hydrogen or (1-3C)alkyl;
R¹ is selected from hydrogen or a group of the formula:
X⁰—X¹-Q¹
wherein
X⁰ is absent or —(CH₂)ₙ—;
X¹ is absent —CO— or —SO₂—;
n is 1, 2 or 3;
when X¹ is absent or —CO— then Q¹ is selected from (1-5C)alkyl, (3-8C)cycloalkyl, aryl, a carbon-linked heterocyclyl, a carbon-linked heteroaryl or —NR⁷R⁸ where R⁷ and R⁸ are each independently selected from methyl or ethyl, or R⁷ and R⁸ are linked so that, together with the nitrogen atom to which they are attached, they form a 4-7 membered heterocyclic ring optionally comprising one, two or three additional heteroatoms selected from N, O or S;
when X¹ is —SO₂— then Q¹ is selected from (1-5C)alkyl, (3-8C)cycloalkyl, phenyl, thiophene or —NR⁷R⁸;
and wherein Q¹ is optionally substituted by one or more substituents selected from halo, trifluoromethyl, trifluoromethoxy, cyano, isocyano, nitro, hydroxy, mercapto, amino, formyl, carboxy, carbamoyl, ureido, (1-4C)alkyl, (2-4C)alkenyl, (2-4C)alkynyl, (1-4C)alkoxy, (1-4C)alkylthio, (1-4C)alkylsulphinyl, (1-4C)alkylsulphonyl, (1-4C)alkylamino, di-[(1-4C)alkyl]amino, (1-4C)alkoxycarbonyl, N-(1-4C)alkylcarbamoyl, N,N-di-[(1-4C)alkyl]carbamoyl, (2-4C)alkanoyl, (2-4C)alkanoyloxy, (2-4C)alkanoylamino, sulphamoyl, N-(1-4C)alkylsulphamoyl, N,N-di-[(1-4C)alkyl]sulphamoyl or a phenyl ring which is optionally further substituted by halo, methoxy, ethoxy, methyl, ethyl, cyano or hydroxy;
R² is selected from the group consisting of:
(i) when R¹ is hydrogen, R² is selected from (3-5C)alkyl, (3-5C)alkenyl, (3-5C)alkynyl, aryl, (3-8C)cycloalkyl, thienyl or a group of the formula:
—CH₂-Q²
wherein Q² is selected from:
(3-8C)cycloalkyl which is optionally substituted with cyano, nitro, fluoro or methyl;
phenyl which is substituted in the ortho or para position (relative to the point of attachment to the —CH₂— group) by cyano, nitro, methyl, —CO₂H and tetrazole and optionally further substituted with cyano, nitro, fluoro, or methyl;
naphthyl which is optionally substituted with cyano, nitro, fluoro or methyl; or
thiophene which is optionally substituted with cyano, nitro, fluoro or methyl;
and
(ii) when R¹ is a substituent group other than hydrogen, R² is selected from (3-5C)alkyl, (3-5C)alkenyl, (3-5C)alkynyl, aryl, (3-8C)cycloalkyl, thienyl or a group of the formula:
—CH₂-Q³
wherein Q³ is selected from phenyl, (3-8C)cycloalkyl, naphthyl or a neutral heteroaryl, each of which is optionally substituted by cyano, nitro, halo, methyl, trifluoromethyl, trifluoromethoxy, isocyano, hydroxy, mercapto, amino, carboxy, carbamoyl, methoxy, methylthio, methylsulphinyl, methylsulphonyl, methylamino, or di-methylamino;
R³, R⁴, R⁵ or R⁶ are each independently selected from hydrogen, halo, (1-5C)alkyl, (3-6C)cycloalkyl, (3-6C)cycloalkyl(1-2C)alkyl, thiophene or aryl, wherein the aryl ring is optionally substituted by halo, methoxy, ethoxy, methyl, ethyl, cyano or hydroxy;
with the proviso that R⁴, R⁵ or R⁶ are all hydrogen when R¹ is hydrogen;
or a pharmaceutically acceptable salt or solvate thereof.

2. The method according to claim 1, for use in the treatment of inflammation, cardiovascular disease (e.g. atherosclerosis), inflammatory pain (both peripheral inflammatory pain and chronic inflammatory pain), allergies and regeneration processes (e.g. physical wounds).

3. The method according to claim 1 having the structural formula II: 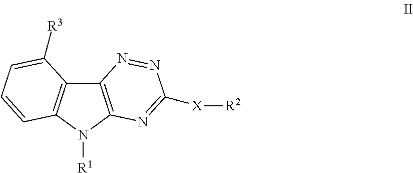
wherein X, R¹, R² and R³ are as defined in claim 1.

4. The method according to claim 1, wherein X is selected from the group consisting of —S—, —SO— and —O—.

5. The method according to claim 1, wherein R¹ is selected from the group consisting of hydrogen and a group of the formula:
X⁰—X¹-Q¹
wherein
X⁰ is absent or —(CH₂)ₙ—;
X¹ is absent, —CO— or —SO₂—;
n is 1 or 2;
when X¹ is absent or —CO— then Q¹ is selected from (1-5C)alkyl, (3-8C)cycloalkyl, phenyl, a carbon-linked heterocyclyl, a carbon-linked heteroaryl or —NR⁷R⁸ where R⁷ and R⁸ are each independently selected from methyl or ethyl, or R⁷ and R⁸ are linked so that, together with the nitrogen atom to which they are attached, they form a 4-6 membered heterocyclic ring optionally comprising one or two additional heteroatoms selected from N, O or S;
when X¹ is —SO₂— then Q¹ is selected from (1-5C)alkyl, (3-8C)cycloalkyl, phenyl, or or —NR⁷R⁸;
and wherein Q¹ is optionally substituted by one or more substituents selected from halo, trifluoromethyl, trifluoromethoxy, cyano, nitro, hydroxy, amino, carboxy, carbamoyl, (1-4C)alkyl, (2-4C)alkenyl, (2-4C)alkynyl, (1-4C)alkoxy, (1-4C)alkylthio, (1-4C)alkylsulphinyl, (1-4C)alkylsulphonyl, (1-4C)alkylamino, di-[(1-4C)alkyl]amino, (1-4C)alkoxycarbonyl, N-(1-4C)alkylcarbamoyl, N,N-di-[(1-4C)alkyl]carbamoyl, (2-4C)alkanoyl, (2-4C)alkanoyloxy, sulphamoyl, N-(1-4C)alkylsulphamoyl, N,N-di-[(1-4C)alkyl]sulphamoyl or a phenyl ring.

6. The method according to claim 5, wherein R¹ is selected from the group consisting of hydrogen and a group of the formula:
X⁰—X¹-Q¹
wherein
X⁰ is absent or —(CH₂)ₙ—;
X¹ is absent, —CO— or —SO₂—;
n is 1, or 2;
when X¹ is absent or —CO— then Q¹ is selected from (1-5C)alkyl, (3-8C)cycloalkyl, phenyl, a carbon-linked heteroaryl or —NR⁷R⁸ wherein R⁷ and R⁸ are linked so that, together with the nitrogen atom to which they are attached, they form a 4-6 membered heterocyclic ring optionally comprising one additional heteroatom selected from N, O or S;
when X¹ is —SO₂— then Q¹ is selected from (1-5C)alkyl, (3-8C)cycloalkyl, phenyl or —NR⁷R⁸;
and wherein Q¹ is optionally substituted by one or more substituents selected from halo, trifluoromethyl, trifluoromethoxy, cyano, nitro, hydroxy, amino, (1-4C)alkyl, (1-4C)alkoxy, (1-4C)alkylthio, (1-4C)alkylsulphinyl, (1-4C)alkylsulphonyl, (1-4C)alkylamino, di-[(1-4C)alkyl]amino, (1-4C)alkoxycarbonyl, (2-4C)alkanoyl, or a phenyl ring;

7. The method according to claim 1, wherein R² is selected from the group consisting of:
(i) when R¹ is hydrogen, R² is selected from (3-5C)alkyl, phenyl, (3-8C)cycloalkyl, thienyl or a group of the formula:
—CH₂-Q²
wherein Q² is selected from:
(3-8C)cycloalkyl which is optionally substituted with cyano, nitro, fluoro or methyl;
phenyl which is optionally substituted in the ortho or para position (relative to the point of attachment to the —CH₂— group) by cyano, nitro, methyl, —CO₂H and tetrazole and optionally further substituted with cyano, nitro, fluoro, or methyl;
thiophene which is optionally substituted with cyano, nitro, fluoro or methyl;
and
(ii) when R¹ is a substituent group other than hydrogen, R² is selected from (3-5C)alkyl, phenyl, (3-8C)cycloalkyl, thienyl or a group of the formula:
—CH₂-Q³
wherein Q³ is selected from phenyl, (3-8C)cycloalkyl, or a 5 or 6-membered neutral heteroaryl, each of which is optionally substituted by cyano, nitro, halo, methyl, trifluoromethyl, trifluoromethoxy, hydroxy, amino, methoxy, methylthio, methylsulphinyl, methylsulphonyl, methylamino or di-methylamino;

8. The method according to claim 7, wherein R² is selected from the group consisting of:
(i) when R¹ is hydrogen, R² is selected from (3-5C)alkyl, or a group of the formula:
—CH₂-Q²
wherein Q² is selected from:
phenyl which is optionally substituted in the ortho or para position (relative to the point of attachment to the —CH₂— group) by cyano, nitro, methyl, —CO₂H and tetrazole and optionally further substituted with cyano, nitro, fluoro, or methyl; and
(ii) when R¹ is a substituent group other than hydrogen, R² is selected from (3-5C)alkyl or a group of the formula:
—CH₂-Q³
wherein Q³ is selected from phenyl, which is optionally substituted by cyano, nitro, halo, methyl, trifluoromethyl, trifluoromethoxy, hydroxy, amino, methoxy, methylthio, methylsulphinyl, methylsulphonyl, methylamino or di-methylamino.

9. The method according to claim 1, wherein R³ is selected from the group consisting of hydrogen, halo, (1-3C)alkyl, (3-6C)cycloalkyl, (3-6C)cycloalkyl(1-2C)alkyl, thiophene and phenyl, wherein any phenyl ring is optionally substituted by halo, methoxy, or methyl.

10. The method according to claim 9, wherein R³ is selected from the group consisting of hydrogen, halo, (1-5C)alkyl and phenyl.

11. A compound of formula I or formula II: 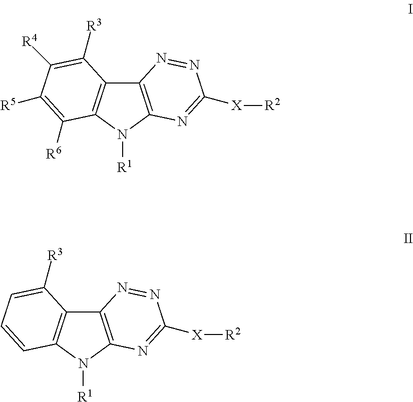
wherein:
X is S or —SO—;
R¹ is a group of the formula:
X⁰—X¹-Q¹
wherein
X⁰ is absent or —(CH₂)—;
X¹ is absent, —CO— or —SO₂—;
n is 1 or 2;
when X¹ is absent then Q¹ is selected from (3-8C)cycloalkyl, aryl, a carbon-linked heterocyclyl, a carbon-linked heteroaryl or —NR⁷R⁸ where R⁷ and R⁸ are each independently selected from methyl or ethyl, or R⁷ and R⁸ are linked so that, together with the nitrogen atom to which they are attached, they form a 4-7 membered heterocyclic ring optionally comprising one, two or three additional heteroatoms selected from N, O or S;
when X¹ is —CO— then Q¹ is selected from (1-5C)alkyl, (3-8C)cycloalkyl, aryl, a carbon-linked heterocyclyl, a carbon-linked heteroaryl or —NR⁷R⁸ where R⁷ and R⁸ are each independently selected from methyl or ethyl, or R⁷ and R⁸ are linked so that, together with the nitrogen atom to which they are attached, they form a 4-7 membered heterocyclic ring optionally comprising one, two or three additional heteroatoms selected from N, O or S;
when X¹ is —SO₂— then Q¹ is selected from (1-5C)alkyl, (3-8C)cycloalkyl, phenyl, thiophene or —NR⁷R⁸;
and wherein Q¹ is optionally substituted by one or more substituents selected from halo, trifluoromethyl, trifluoromethoxy, cyano, isocyano, nitro, hydroxy, mercapto, amino, formyl, carboxy, carbamoyl, ureido, (1-4C)alkyl, (2-4C)alkenyl, (2-4C)alkynyl, (1-4C)alkoxy, (1-4C)alkylthio, (1-4C)alkylsulphinyl, (1-4C)alkylsulphonyl, (1-4C)alkylamino, di-[(1-4C)alkyl]amino, (1-4C)alkoxycarbonyl, N-(1-4C)alkylcarbamoyl, N,N-di-[(1-4C)alkyl]carbamoyl, (2-4C)alkanoyl, (2-4C)alkanoyloxy, (2-4C)alkanoylamino, sulphamoyl, N-(1-4C)alkylsulphamoyl, N,N-di-[(1-4C)alkyl]sulphamoyl or a phenyl ring which is optionally further substituted by halo, methoxy, ethoxy, methyl, ethyl, cyano or hydroxy;
R² is selected from the group consisting of:
(i) when R¹ is hydrogen, R² is a group of the formula:
—CH₂-Q²
wherein Q² is selected from:
(3-8C)cycloalkyl which is optionally substituted with cyano, nitro, fluoro or methyl;
phenyl which is substituted in the ortho or para position (relative to the point of attachment to the —CH₂— group) by cyano, nitro, methyl, —CO₂H and tetrazole and optionally further substituted with cyano, nitro, fluoro, or methyl;
naphthyl which is optionally substituted with cyano, nitro, fluoro or methyl; or
thiophene which is optionally substituted with cyano, nitro, fluoro or methyl;
and
(ii) when R¹ is a substituent group other than hydrogen, R² is a group of the formula:
—CH₂-Q³
wherein Q³ is selected from phenyl, (3-8C)cycloalkyl, naphthyl or a neutral heteroaryl, each of which is optionally substituted by cyano, nitro, halo, methyl, trifluoromethyl, trifluoromethoxy, isocyano, hydroxy, mercapto, amino, carboxy, carbamoyl, methoxy, methylthio, methylsulphinyl, methylsulphonyl, methylamino, or di-methylamino;
R³, R⁴, R⁵, R⁶ are as defined in claim 1;
or a pharmaceutically acceptable salt or solvate thereof.

12. The compound according to claim 11, wherein the compound of formula I is selected from the group consisting of:
3-((2′-nitrobenzyl)thio)-5H-[1,2,4]triazino[5,6-b]indole;
3-((2′-cyanobenzyl)thio)-5H-[1,2,4]triazino[5,6-b]indole;
2′-(((5-benzyl-5H-[1,2,4]triazino[5,6-b]indol-3-yl)thio)methyl)benzonitrile;
5-benzyl-3-(2′-fluorobenzylthio)-5H-[1,2,4]triazino[5,6-b]indole;
2′(((5-(4″-chlorobenzyl)-5H-[1,2,4]triazino[5,6-b]indol-3-yl)thio)methyl)benzonitrile;
5-(4″-chlorobenzyl)-3-((2′-nitrobenzyl)thio)-5H-[1,2,4]triazino[5,6-b]indole;
(3-((2′-nitrobenzyl)thio)-5H-[1,2,4]triazino[5,6-b]indol-5-yl)(phenyl)methanone;
2′-(((9-bromo-5H-[1,2,4]triazino[5,6-b]indol-3-yl)thio)methyl)benzonitrile;
9-bromo-3-((2′-nitrobenzyl)thio)-5H-[1,2,4]triazino[5,6-b]indole;
2′-(((5-(2″-nitrobenzyl)-5H-[1,2,4]triazino[5,6-b]indol-3-yl)thio)methyl)benzonitrile;
3-((2′,4′-dinitrobenzyl)thio)-5H-[1,2,4]triazino[5,6-b]indole;
3-((2′-fluoro-6′-nitrobenzyl)thio)-5H-[1,2,4]triazino[5,6-b]indole;
5-(2″-fluorobenzyl)-3-((2′-nitrobenzyl)thio)-5H-[1,2,4]triazino[5,6-b]indole;
5-benzyl-3-((2′-nitrobenzyl)thio)-5H-[1,2,4]triazino[5,6-b]indole;
3-((2′-methylbenzyl)thio)-5H-[1,2,4]triazino[5,6-b]indole;
2′-(((5-(4″-dimethylamino)benzoyl)-5H-[1,2,4]triazino[5,6-b]indol-3-yl)thio)methyl)benzonitrile;
2′-(((5-(3″,4″-dimethoxybenzoyl)-5H-[1,2,4]triazino[5,6-b]indol-3-yl)thio)methyl)benzonitrile;
2′-(((5-(cyclohexanecarbonyl)-5H-[1,2,4]triazino[5,6-b]indol-3-yl)thio)methyl)benzonitrile;
2′((5-(3″-trifluoromethyl)benzoyl)-5H-[1,2,4]triazino[5,6-b]indol-3-yl)thio)methyl)benzonitrile;
2′-(((5-(4′-fluorobenzoyl)-5H-[1,2,4]triazino[5,6-b]indol-3-yl)thio)methyl)benzonitrile;
2′-(((5-(4″-(trifluoromethoxy)benzoyl)-5H-[1,2,4]triazino[5,6-b]indol-3-yl)thio)methyl)benzonitrile;
2′-(((5-(cyclopentanecarbonyl)-5H-[1,2,4]triazino[5,6-b]indol-3-yl)thio)methyl)benzonitrile;
2′-(((5-(furan-2″-carbonyl)-5H-[1,2,4]triazino[5,6-b]indol-3-yl)thio)methyl)benzonitrile;
2′-(((5-(isoxazole-5″-carbonyl)-5H-[1,2,4]triazino[5,6-b]indol-3-yl)thio)methyl)benzonitrile;
2′-(((5-(benzoyl-5H-[1,2,4]triazino[5,6-b]indol-3-yl)thio)methyl)benzonitrile;
methyl 4″-(3-((2′-cyanobenzyl)thio)-5H-[1,2,4]triazino[5,6-b]indol-5-yl)-4″-oxobutanoate;
methyl 5″-(3-((2′-cyanobenzyl)thio)-5H-[1,2,4]triazino[5,6-b]indol-5-yl)-5″-oxopentanoate;
3-(butylthio)-5H-[1,2,4]triazino[5,6-b]indole;
2′(((5-acetyl-5H-[1,2,4]triazino[5,6-b]indol-3-yl)thio)methyl)benzonitrile;
2′-(((5-ethyl-5H-[1,2,4]triazino[5,6-b]indol-3-yl)thio)methyl)benzonitrile;
2′-(((5-butyl-5H-[1,2,4]triazino[5,6-b]indol-3-yl)thio)methyl)benzonitrile;
2′-(((5H-[1,2,4]triazino[5,6-b]indol-3-yl)sulfinyl)methyl)benzonitrile;
3-((4′-nitrobenzyl)thio)-5H-[1,2,4]triazino[5,6-b]indole;
2′-(((5-(phenylsulfonyl)-5H-[1,2,4]triazino[5,6-b]indol-3-yl)thio)methyl)benzonitrile;
2′-(((5-(morpholine-4″-carbonyl)-5H-[1,2,4]triazino[5,6-b]indol-3-yl)thio)methyl)benzonitrile;
2′-(((5-(cyclohexylmethyl)-5H-[1,2,4]triazino[5,6-b]indol-3-yl)thio)methyl)benzonitrile;
2′-(((5-(4″-fluorobenzyl)-5H-[1,2,4]triazino[5,6-b]indol-3-yl)thio)methyl)benzonitrile;
2′-((5-(2″-phenylcyclopropanecarbonyl)-5H-[1,2,4]triazino[5,6-b]indol-3-ylthio)methyl)benzonitrile;
2′-((5-(2-cyclopropyl-2-oxoethyl)-5H-[1,2,4]triazino[5,6-b]indol-3-ylthio)methyl)benzonitrile;
2′-((5-(2″-methylcyclopropanecarbonyl)-5H-[1,2,4]triazino[5,6-b]indol-3-ylthio)methyl)benzonitrile;
2′-((5-(cyclopropylmethyl)-5H-[1,2,4]triazino[5,6-b]indol-3-ylthio)methyl)benzonitrile;
2′-(((5-(cyclopropanecarbonyl)-5H-[1,2,4]triazino[5,6-b]indole-3-yl)thio)methyl)benzonitrile;
2′-(((9-bromo-5-(cyclopropylmethyl)-5H-[1,2,4]triazino[5,6-b]indol-3-yl)thio)methyl)benzonitrile;
2′-(((9-bromo-5-(cyclopropanecarbonyl)-5H-[1,2,4]triazino[5,6-b]indol-3-yl)thio)methyl)benzonitrile;
methyl-4″-(9-bromo-3-((2′-cyanobenzyl)thio)-5H-[1,2,4]triazino[5,6-b]indol-5-yl)-4″-oxobutyrate;
2′-(((5H-[1,2,4]triazino[5,6-b]indol-3-yl)thio)methyl)benzoic acid;
3-(((2-(2H-tetrazol-5-yl)benzyl)thio)-5H-[1,2,4]triazino[5,6-b]indole;
2″-(((9-(o-tolyl)-5H-[1,2,4]triazino[5,6-b]indol-3-yl)thio)methyl)benzonitrile;
2″-(((9-(4′-methoxyphenyl)-5H-[1,2,4]triazino[5,6-b]indol-3-yl)thio)methyl)benzonitrile;
2″-(((9-(thiophen-3′-yl)-5H-[1,2,4]triazino[5,6-b]indol-3-yl)thio)methyl)benzonitrile;
2″-(((9-cyclopropyl-5H-[1,2,4]triazino[5,6-b]indol-3-yl)thio)methyl)benzonitrile;
or a pharmaceutically acceptable salt or solvate thereof.

13. A pharmaceutical composition comprising a compound according to claim 11, or a pharmaceutically acceptable salt or solvate thereof, in admixture with a pharmaceutically acceptable diluent or carrier.

14-16. (canceled)

17. A method of synthesising a compound of formula I according to claim 1 in which X is S or O, said method comprising:
(i) the reaction of a compound of formula IA: 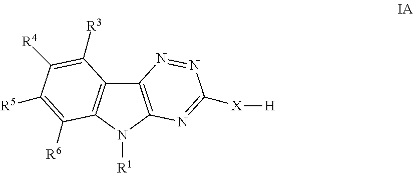
wherein X is S or O and R¹, R³, R⁴, R⁵ and R⁶ are each as defined in claim 1;
with a compound of formula IB:
R²-L IB
wherein R² is as defined in claim 1 and L is a leaving group;
in a suitable solvent.

18. A method of synthesising a compound of formula I according to claim 1 in which X is SO, said method comprising:
(i) synthesising a compound in which X is S by the method defined in claim 17; and
(ii) oxidising the compound in which X is S with a suitable oxidising agent to form a compound of formula I in which X is SO.

19. A method of synthesising a compound of formula I according to claim 1 in which R¹ is a substituent other than hydrogen, said method comprising:
(i) the reaction of a compound of formula IC: 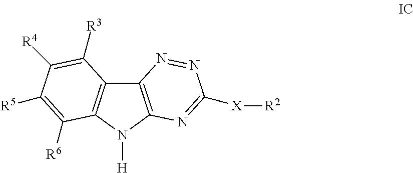
wherein X, and R², R³, R⁴, R⁵ and R⁶ are each as defined in claim 1 hereinbefore;
with a compound of formula ID:
R¹-L ID
wherein R² is as defined in claim 1 and L is a suitable leaving group;
in a suitable solvent.

20. A compound having the structural formula II: 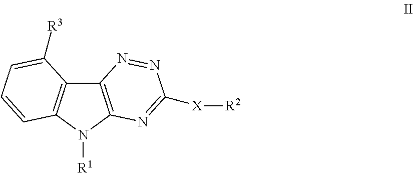
wherein X, R¹, R² and R³ are as defined in claim 11.

21. A compound according to claim 11, wherein R³, R⁴, R⁵ or R⁶ are selected from the group consisting of hydrogen, halo, (1-3C)alkyl, (3-6C)cycloalkyl, (3-6C)cycloalkyl(1-2C)alkyl, thiophene and phenyl, wherein any phenyl ring is optionally substituted by halo, methoxy, or methyl.

22. The compound according to claim 21, wherein R³, R⁴, R⁵ or R⁶ are selected from the group consisting of hydrogen, halo, (1-5C)alkyl and phenyl.
